(19) United States
(12) Patent Application Publication
(10) Pub. No.: US 2023/0296610 A1
(43) Pub. Date: Sep. 21, 2023

(54) PREDICTING ADVERSE EVENTS FROM IMMUNOTHERAPY

(71) Applicant: Kite Pharma, Inc., Foster City, CA (US)

(72) Inventors: Sabina Adhikary, Venice, CA (US); Adrian I. Bot, Beverly Hills, CA (US); Justin Szu-ting Chou, Los Angeles, CA (US); Qinghua Song, San Francisco, CA (US); Kelly Speth, Ann Arbor, MI (US); Gayatri R. Tiwari, Camarillo, CA (US)

(21) Appl. No.: 18/168,791

(22) Filed: Feb. 14, 2023

Related U.S. Application Data
(60) Provisional application No. 63/310,438, filed on Feb. 15, 2022.

(51) Int. Cl. G01N 33/574; A61K 31/675; A61K 31/7076; A61K 45/06; A61K 39/00; C07K 16/28; C07K 14/725

(52) U.S. Cl. CPC G01N 33/57488; A61K 31/675; A61K 31/7076; A61K 45/06; A61K 39/4611; A61K 39/4631; C07K 16/2803; C07K 14/7051; A61K 39/464412; C07K 2317/622; A61K 2039/505

(57) ABSTRACT

The disclosure relates to methods of diagnosis and prognosis, compositions for immunotherapies, methods of improving said compositions, and immunotherapies using the same (e.g., T cells, non-T cells, TCR-based therapies, CAR-based therapies, bispecific T-cell engagers (BiTEs), and/or immune checkpoint blockade).

### CROSS REFERENCE TO RELATED APPLICATIONS

This application claims priority to U.S. Provisional Patent Application No. 63/310,438 filed on Feb. 15, 2022, the entire contents of which are hereby incorporated by reference.

### FIELD

The disclosure relates to methods of diagnosis and prognosis, compositions for immunotherapies, and immunotherapies using the same.

### BACKGROUND

Human cancers are by their nature comprised of normal cells that have undergone a genetic or epigenetic conversion to become abnormal cancer cells. In doing so, cancer cells begin to express proteins (including, but not limited to, antigens) that are distinct from those expressed by normal cells. These aberrant tumor antigens may be used by the body's innate immune system to specifically target and kill cancer cells. However, cancer cells employ various mechanisms to prevent immune cells, such as T and B lymphocytes, from successfully targeting cancer cells.

Human T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. For example, chimeric antigen receptors (CARs) and T Cell Receptors (TCRs), which comprise binding domains capable of interacting with a particular tumor antigen, allow T cells to target and kill cancer cells that express the particular tumor antigen. There is a need to understand how pre-treatment patient attributes (e.g., serum proteomic profile, tumor burden, tumor genetic and proteomic profile) influence treatment outcomes, including safety outcomes of immunotherapy.

### SUMMARY

It is to be understood that the disclosure is not limited in its application to the details set forth in the following embodiments, claims, description and figures. The disclosure is capable of other embodiments and of being practiced or carried out in numerous other ways.

Provided herein are methods that involve assessing particular parameters, e.g., expression of specific biomarkers or analytes, that can be correlated with an outcome, such as a therapeutic outcome, including a response, such as a complete response (CR) or a partial response (PR); or a safety outcome, such as a development of a toxicity, for example, neurotoxicity or CRS, after administration of immunotherapy (e.g., cell therapy). Also provided are methods to assess the likelihood of response and/or likelihood of risk of toxicity, based on assessment of the parameters, such as expression of biomarkers or analytes in the patient. Also provided are immunotherapies (e.g., T cells, non-T cells, TCR-based therapies, CAR-based therapies, bispecific T-cell engagers (BiTEs), and/or immune checkpoint blockade), including methods and uses of cells (e.g., engineered T cells) and/or compositions thereof, for the treatment of subjects having a disease or condition, which generally is or includes a cancer or a tumor, such as a leukemia or a lymphoma. In some aspects, the methods and uses provide for or achieve improved response and/or more durable responses or efficacy and/or a reduced risk of toxicity or other side effects, in subjects treated with some methods, as compared to certain alternative methods. In some embodiments, the methods comprise the administration of specified numbers or relative numbers of the engineered cells, the administration of defined ratios of particular types of the cells, treatment of particular patient populations, such as those having a particular risk profile, staging, and/or prior treatment history, administration of additional therapeutic agents and/or combinations thereof.

In one embodiment, the disclosure provides that baseline (pre-conditioning) serum levels of certain protein associated with metabolic processes and leukocyte activation correlate positively with, and can be biomarkers for, poor prognosis factors for immunotherapy including international prognostic index and baseline tumor burden. In one embodiment, the metabolic process biomarkers are PAG1, SOD2, and KYNU. In one embodiment, the leukocyte activation biomarkers are AREG, NUB1, HLA-DRA, LY9, SERNPINB9, OSMR, IL1A, REG3A, and REG1B. In one embodiment, the biomarker levels are analyzed by Olink panels. In one embodiment, the immunotherapy is T cell therapy. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells In one embodiment, the immunotherapy is CAR T cell or TCR T cell therapy. In one embodiment, the immunotherapy is anti-CD19 CAR T cell therapy.

In one embodiment, the disclosure provides that baseline (pre-conditioning) serum levels of certain proteins associated with IL1/IL6 pathway correlate positively with, and can be biomarkers for, Grade 3+ cytokine release syndrome (CRS) after immunotherapy. In one embodiment, the biomarkers are IL1A and OSMR. In one embodiment, the disclosure provides that baseline (pre-conditioning) serum levels of certain proteins associated with inflammatory endothelial markers correlate positively with, and can be biomarkers for, both Grade 3+ CRS and Grade 3+ neurologic events (NE) after immunotherapy. In one embodiment, the inflammatory endothelial markers are ACE2, CEACAM1, ICAM2, and ADAM15. In one embodiment, the immunotherapy is T cell therapy. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells In one embodiment, the immunotherapy is CAR T cell or TCR T cell therapy. In one embodiment, the immunotherapy is anti-CD19 CART cell therapy.

In one embodiment, the disclosure provides that baseline serum levels of MET, OSMR, ACE2, ACY1, BILDIR correlate positively with, and can be biomarkers for, Grade 3+CRS and NE after immunotherapy. In one embodiment, the disclosure provides that day 0 (i.e., immediately prior to immunotherapy and after conditioning therapy) baseline serum levels of CCL16, CELA3A, MEGF9, MFAP3, REG1B, AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ITM2A, ICOSLG, CGA, CRNN, LY96, AST, and VAMP5 correlate positively with, and can be biomarkers for, Grade 3+ CRS and NE after immunotherapy. In one embodiment, the disclosure provides that the value for the fold change between day 0 and baseline serum levels of GPA33, EPCAM, and CRNN correlate positively with, and can be biomarkers for, Grade 3+ CRS and NE after immunotherapy. In one embodiment, serum levels of one or more of these proteins correlate positively with, and can be biomarkers for, preconditioning and/or immune activation induced stress after pre-conditioning regimen further exacerbated by immunotherapy. In one embodiment, the conditioning therapy comprises cyclophosphamide and fludarabine.

The following are non-limiting embodiments of the disclosure.

An embodiment of the disclosure relates to a method of determining a risk of an adverse event upon administration of an immunotherapy to a cancer subject in need thereof, the method comprising: quantifying a baseline serum level from the cancer subject of one or more biomarkers selected from REG3A, KYNU, OSMR, NELL2, and MET; or quantifying a day 0 serum level from the cancer subject of one or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5; and determining the risk of the adverse event in the cancer subject based on the step of quantifying the baseline serum level of the one or more biomarkers or the step of quantifying the day 0 serum level from the cancer subject of the one or more biomarkers. In such an embodiment, at least one of an increased baseline serum level of the one or more biomarkers versus a control baseline serum level of the one or more biomarkers and/or an increased day 0 serum level of the one or more biomarkers versus a control day 0 serum level of the one or more biomarkers is correlated with an increased risk of the adverse event in the cancer subject. In such an embodiment, the adverse event is one or more of an immune activation induced stress, a Grade 3+ CRS, and a Grade 3+ NE. In such an embodiment, a baseline serum level refers to a serum level of the one or more indicated biomarkers, where the serum sample was collected prior to administration of a pre-conditioning regimen to the cancer subject. In such an embodiment, a day 0 serum level refers to a serum level of the one or more indicated biomarkers, where the serum sample was collected following administration of a conditioning therapy to the cancer subject, but prior to administration of the immunotherapy to the cancer subject.

An embodiment of the disclosure relates to the method above, where the method comprises quantifying a baseline serum level from the cancer subject of one or more biomarkers selected from REG3A, KYNU, OSMR, NELL2, and MET; and quantifying a day 0 serum level from the cancer subject of one or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5; and determining the risk of the adverse event in the cancer subject based on the step of quantifying the baseline serum level of the one or more biomarkers and the step of quantifying the day 0 serum level from the cancer subject of the one or more biomarkers.

An embodiment of the disclosure relates to any of the methods above, where the method comprises quantifying a baseline serum level from the cancer subject of two or more biomarkers selected from REG3A, KYNU, OSMR, NELL2, and MET.

An embodiment of the disclosure relates to any of the methods above, where the method comprises quantifying a day 0 serum level from the cancer subject of two or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5.

An embodiment of the disclosure relates to any of the methods above, where the control baseline serum level of the one or more biomarkers is a historically observed baseline serum level of the one or more biomarkers not previously observed to be associated with an onset of the adverse event, and wherein the control day 0 serum level of the one or more biomarkers is a historically observed day 0 serum level of the one or more biomarkers not previously observed to be associated an onset of the adverse event.

An embodiment of the disclosure relates to any of the methods above, the quantifying of the baseline (pre-conditioning) serum level from the cancer subject of one or more biomarkers is KYNU, and the quantifying the day 0 serum level from the cancer subject of one or more biomarkers is selected from ACE2, ADAM15, BSG, CEACAM1, HLA-DRA, ICAM2, LY9, LY96, OSMR, REG3A, SERPINB9, SOD2, and VAMP5.

An embodiment of the disclosure relates to any of the methods above, where the cancer subject is determined to have an increased risk of the adverse event if the baseline serum level of one or more biomarkers selected from MET, OSMR, NELL2, REG3A, KYNU and the day 0 serum level of one or more biomarkers selected from AREG, CKAP4, CXCL1, EIF5A, IL1A, KIFBP, KIRREL2, NUB1, PAG1, PCHD17, STK11, TGFB1, ACE2, ADAM15, BSG, CEACAM1, HLA-DRA, ICAM2, LY9, LY96, OSMR, REG3A, SERPINB9, SOD2, VAMP5, CCL16, CELA3A, MEGF9, MFAP3, REG1B is above a predetermined normalized protein expression range for the one or more biomarkers, where the predetermined normalized protein expression range for the one or more biomarkers are 0.56-0.75 for MET, 0.62-0.77 for OSMR (baseline), 0.14-0.50 for NELL2, 1.80-2.19 for REG3A (baseline), 1.60-1.98 for KYNU, 0.84-1.25 for CCL16, 1.37-2.01 for CELA3A, 0.26-0.48 for MEGF9, 0.33-0.49 for MFAP3, 1.49-1.99 for REG1B, 0.88-1.22 for AREG, 0.73-0.96 for BSG, 1.83-2.31 for CKAP4, 3.48-3.91 for CXCL1, 0.09-0.23 for EIF5A, 0.46-0.81 for IL1A, 1.36-1.66 for KIFBP, 1.62-2.13 for KIRREL2, 1.39-1.77 for NUB1, 0.65-0.79 for OSMR (day 0), 2.16-2.74 for PAG1, 1.03-1.32 for PCDH17, 1.09-1.35 for STK11, 1.20-1.65 for ACE2, 1.09-1.31 for ADAM15, 0.68-0.90 for CEACAM1, 1.13-1.41 for HLA-DRA, 1.21-1.43 for ICAM2, 0.11-0.49 for LY9, 2.34-2.88 for REG3A (day 0), 2.12-2.30 for SERPINB9, 1.37-1.86 for SOD2, 0.54-0.70 for LY96, 1.51-1.67 for TGFB1, and 1.30-1.73 for VAMP5. In such an embodiment, the normalized protein expression range is calculated at least in part based on the mean, standard deviation (SD), and standard error of the mean (SE) of the expression data for each respective biomarker. IN such an analysis, a ‘0’ refers to a group with “No” toxicity, while a 1′ refers to a group with “Yes” toxicity. SE is calculated as SD/sqrt(n), where ‘n’ represents the sample size in each group. Lower and upper bounds are calculated as Mean·NPX+/−1*SE (for the group with toxicity). Each of the calculated ranges depicted above for each of the mentioned biomarkers represents a reasonable range for a possible cut-point for each biomarker, where observed values above the cut-point indicate that the cancer subject may experience Grade 3+ cytokine release syndrome and/or Grade 3+ neurologic events within 5 days following treatment with the immunotherapy.

An embodiment of the disclosure relates to any of the methods above, further comprising one or more of treating the cancer subject with a modified pre-conditioning regimen versus a standard pre-conditioning regimen, not administering the immunotherapy to the cancer subject, administering the immunotherapy to the cancer subject with a modified dose versus a standard dose, or administering the immunotherapy to the cancer subject in combination with an agent for mediating an immune activation induced stress, a Grade 3+ CRS, and/or a Grade 3+ NE, if the cancer subject is determined to have an increased risk of an adverse event.

An embodiment of the disclosure relates to any of the methods above, where the modified pre-conditioning regimen comprises administering to the cancer subject a modified dose of at least one of cyclophosphamide and fludarabine instead of a predetermined dose of cyclophosphamide or fludarabine administered to a control subject.

An embodiment of the disclosure relates to any of the methods above, further comprising administering the immunotherapy with a combination therapy comprising an agent that reduces cytokine induction and/or endothelial cells disruption if the cancer subject is determined to have an increased risk of an adverse event.

An embodiment of the disclosure relates to any of the methods above, where the agent reduces cytokine induction.

An embodiment of the disclosure relates to any of the methods above, where the agent is administered to the cancer subject prior to administration of the immunotherapy, before a peak expansion of the immunotherapy, or at the peak expansion of the immunotherapy.

An embodiment of the disclosure relates to any of the methods above, where the agent is selected from an anti-IL-1 molecule, a T-cell activation inhibitor, a JAK inhibitor, an anti-GM-CSF molecule, an anti-TNF molecule, an Ang2 inhibitor, an anti-angiogenic therapy, and an anti-IFNg molecule.

An embodiment of the disclosure relates to any of the methods above, the immunotherapy is a CAR T cell therapy, a TCR T cell therapy, a tumor infiltrating lymphocytes (TIL) cell therapy, or a bispecific T-cell engagers (BiTEs) therapy.

An embodiment of the disclosure relates to any of the methods above, where the immunotherapy is autologous or allogeneic.

An embodiment of the disclosure relates to any of the methods above, where the immunotherapy is CAR T or TCR T cell therapy that recognizes a target antigen.

An embodiment of the disclosure relates to any of the methods above, where the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C(TnC Al), thyroglobulin, tumor stromal antigens, vascular endothelial growth factor receptor-2 (VEGFR2), virus-specific surface antigen such as an HIV-specific antigen (such as HIV gp120), GPC3 (Glypican 3), as well as any derivate or variant of these antigens.

An embodiment of the disclosure relates to any of the methods above, where the cancer subject has a cancer or tumor selected from a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

An embodiment of the disclosure relates to any of the methods above, where the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

An embodiment of the disclosure relates to any of the methods above, where the immunotherapy is selected from axicabtagene ciloleucel, brexucabtagene autoleucel, tisagenlecleucel, lisocabtagene maraleucel, and bb2121.

An embodiment of the disclosure relates a method of determining a risk of an adverse event upon administration of an immunotherapy to a cancer subject in need thereof, the method comprising: determining baseline and/or day 0 serum levels of a plurality of biomarkers from the cancer subject; determining a weight for each of the plurality of biomarkers at least in part by inputting the baseline serum levels of the plurality of biomarkers into an algorithm; and determining a probability of risk value of the adverse event based at least in part on the weight for each of the plurality of biomarkers. In such an embodiment, if the probability of risk value is at least as great as a predetermined cut-off value, then the subject has an increased risk of the adverse event versus a control group, and the plurality of biomarkers comprise ACE2, IL1A, SERPINB9, and LY96. In such an embodiment, a baseline serum level refers to a serum level of the one or more indicated biomarkers, where the serum sample was collected prior to administration of a pre-conditioning regimen to the cancer subject. In such an embodiment, a day 0 serum level refers to a serum level of the one or more indicated biomarkers, where the serum sample was collected following administration of a conditioning therapy to the cancer subject, but prior to administration of the immunotherapy to the cancer subject.

An embodiment of the disclosure relates to the method above, where the predetermined cut-off is a Youden cut-off value of between 0.10-0.35.

An embodiment of the disclosure relates to any of the methods above, where the algorithm comprises a logical regression model, a random forest algorithm, or a regularized gradient boosting framework algorithm.

Additional embodiments of the disclosure follow below:

A method of predicting inflammation induced stress and/or predicting Grade 3+CRS and/or Grade 3+ NE upon administration of immunotherapy to a cancer subject in need thereof, the method comprising quantifying pre-treatment:
(i) baseline (pre-conditioning) serum levels of one or more biomarkers selected from ACE2, ACY1, OSMR, BILDIR, and MET;
(ii) day 0 (after conditioning and prior to immunotherapy) serum levels of one or more biomarkers selected from REG1B, MEGF9, CCL16, CELA3A, and MFAP3;
(iii) day 0 (after conditioning and prior to immunotherapy) serum levels of one or more IL1A, REG3A, ICOSLG, ITM2A, CGA, VAMP5, REG1B, LY9, HLA-DRA, LY96, CEACAM1, SOD2, ADA2, BSG, EIF5A, MEGF9, OSMR, PCDH17, ADAM15, ICAM2, SERPINB9, CCL16, KIRREL2, ACE2, AST, CELA3A, MFAP3, and then the rest, AREG, CKAP4, CXCL1, KIFBP, NUB1, PAG1, and STK11;
(iv) baseline (pre-conditioning) serum levels of one or more biomarkers associated with the IL1/IL6 pathway (e.g. IL1A and OSMR); and
(v) baseline (pre-conditioning) serum levels of certain proteins associated with inflammatory endothelial markers (e.g., CEACAM1, ACE2, ICAM2, and ADAM15);
(vi) the value for the fold change between day 0 and baseline serum levels of CRNN, EPCAM, and GPA33;
   wherein the higher the serum level of these protein biomarkers, or the fold change between baseline and day 0 serum levels, the higher the immune activation induced stress and/or, the greater the likelihood that the subject will develop Grade 3+ CRS and/or Grade 3+ NE after immunotherapy;
(vii) or when the patient has low level baseline serum levels of FGF21, or day 0 levels of one or more of CGA, ICOSLG, ITM2A, the greater the likelihood that the subject will develop Grade 3+ CRS and/or Grade 3+ NE after immunotherapy.

The method above, wherein when the baseline serum level of one or more biomarkers selected from MET, OSMR, ACE2, ACYL, FGF21, BILDIR; day 0 CCL16, CELA3A, MEGF9, MFAP3, REG1B, AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ICOSLG, ITM2A, CGA, CRNN, LY96, AST, and VAMP5; fold change between day 0 and baseline GPA33, EPCAM, and CRNN; is above or below, as respectively marked, the following values for each of the respective proteins: baseline 0.56-0.75 (MET, above), 0.62-0.77 (OSMR, above), 0.65-0.98 (ACE2, above), 0.66-1.10 (ACYL, above), 1.58-2.25 (FGF21, below), 4.12-5.50 (BILDIR, above), day 0 0.84-1.25 (CCL16, above), 1.37-2.01 (CELA3A, above), 0.26-0.48 (MEGF9, above), 0.33-0.49 (MFAP3, above), 1.49-1.99 (REG1B, above), 0.88-1.22 (AREG, above), 0.73-0.96 (BSG, above) 1.83-2.31 (CKAP4, above), 3.48-3.91 (CXCL1, above), 0.09-0.23 (EIF5A, above), 0.46-0.81 (IL1A, above), 1.36-1.66 (KIFBP, above), 1.62-2.13 (KIRREL2, above), 1.39-1.77 (NUB1, above), 0.65-0.79 (OSMR, above), 2.16-2.74 (PAG1, above), 1.03-1.32 (PCDH17, above), 1.09-1.35 (STK11, above), 1.20-1.65 (ACE2, above), 1.09-1.31 (ADAM15, above), 0.69-0.90 (CEACAM1, above), 1.13-1.41 (HLA-DRA, above), 1.21-1.43 (ICAM2, above), 0.11-0.49 (LY9, above), 2.34-2.88 (REG3A, above), 2.12-2.30 (SERPINB9, above), 1.37-1.86 (SOD2, above), 0.70-0.98 (ADA2, above), 0.74-1.01 (ITM2A, below), 0.45-0.56 (ICOSLG, below), 1.12-1.50 (CGA, above), −0.48-0.08 (CRNN, above), 0.54-0.70 (LY96, above), 42.12-65.80 (AST, above), and 1.30-1.73 (VAMP5, above), and fold change between day 0 and baseline −1.36-(−0.60) (GPA33, below), −0.82-(−0.39) (EPCAM, below), and −0.27-0.16 (CRNN, above); the subject has a greater likelihood of developing Grade 3+ CRS and/or Grade 3+ NE after immunotherapy than a subject having baseline serum levels that are below or above, respectively, the preceding recited values. In some such embodiments, the recited baseline serum levels are measured by Olink. In some such embodiments, the subject has a greater likelihood of developing Grade 3+ CRS and/or Grade 3+ NE after immunotherapy than a subject having baseline serum levels that are below or above the median baseline serum levels, respectively. In such an embodiment, changes between baseline and Day 0 biomarker levels might be indicative of a systemic dysregulation in the subject as a result of any conditioning chemotherapy regimen, which in turn could predispose the subject to toxicity following administration of an immunotherapy (e.g. a CAR T-cell therapy).

The method above, wherein the measured serum levels of the biomarkers are used to guide subsequent decisions related to the immunotherapy administered to the subject, including but not limited to the conditioning regimen, whether to administer immunotherapy, what type of immunotherapy to administer, what dosage/dosage regimen of immunotherapy to administer, what conditioning protocol, and/or what agent(s) should be administered to the patient prior to, after, and/or during immunotherapy administration (e.g., CAR T cells) to improve management and/or reduce Grade 3+ CRS and/or Grade 3+ NE in the subject.

The method above, wherein the higher the levels of the one or more of the biomarkers or the fold change, the more conservative the conditioning regimen and immunotherapy, etc.

A method to stratify patients having greater likelihood of developing high grade adverse events, the method comprising administering immunotherapy in combination with an agent(s) that reduces immune activation and/or endothelial cells disruption, wherein the combination therapy reduces cytokine induction and/or wherein the combination therapy reduces endothelial cell disruption, wherein the patient is selected for combination therapy when the patient has high level of serum markers, estimated by measuring the protein level of one or more of baseline MET, OSMR, ACE2, ACY1, BILDIR, day 0 CCL16, CELA3A, MEGF9, MFAP3, REG1B, AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, CRNN, LY96, AST, and VAMP5, fold change between day 0 and baseline CRNN in the blood; or when the patient has low level of serum markers, estimated by measuring the protein level of one or more of baseline FGF21, day 0 CGA, ICOSLG, ITM2A, and fold change between day 0 and baseline GPA33 and EPCAM.

The method above, wherein when the baseline serum level of one or more biomarkers selected from MET, OSMR, ACE2, ACY1, FGF21, BILDIR; day 0 CCL16, CELA3A, MEGF9, MFAP3, REG1B, AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ICOSLG, ITM2A, CGA, CRNN, LY96, AST, and VAMP5; fold change between day 0 and baseline GPA33, EPCAM, and CRNN; is above or below, as respectively marked, the following values for each of the respective proteins: baseline 0.56-0.75 (MET, above), 0.62-0.77 (OSMR, above), 0.65-0.98 (ACE2, above), 0.66-1.10 (ACYL, above), 1.58-2.25 (FGF21, below), 4.12-5.50 (BILDIR, above), day 0 0.84-1.25 (CCL16, above), 1.37-2.01 (CELA3A, above), 0.26-0.48 (MEGF9, above), 0.33-0.49 (MFAP3, above), 1.49-1.99 (REG1B, above), 0.88-1.22 (AREG, above), 0.73-0.96 (BSG, above) 1.83-2.31 (CKAP4, above), 3.48-3.91 (CXCL1, above), 0.09-0.23 (EIF5A, above), 0.46-0.81 (IL1A, above), 1.36-1.66 (KIFBP, above), 1.62-2.13 (KIRREL2, above), 1.39-1.77 (NUB1, above), 0.65-0.79 (OSMR, above), 2.16-2.74 (PAG1, above), 1.03-1.32 (PCDH17, above), 1.09-1.35 (STK11, above), 1.20-1.65 (ACE2, above), 1.09-1.31 (ADAM15, above), 0.69-0.90 (CEACAM1, above), 1.13-1.41 (HLA-DRA, above), 1.21-1.43 (ICAM2, above), 0.11-0.49 (LY9, above), 2.34-2.88 (REG3A, above), 2.12-2.30 (SERPINB9, above), 1.37-1.86 (SOD2, above), 0.70-0.98 (ADA2, above), 0.74-1.01 (ITM2A, below), 0.45-0.56 (ICOSLG, below), 1.12-1.50 (CGA, above), −0.48-0.08 (CRNN, above), 0.54-0.70 (LY96, above), 42.12-65.80 (AST, above), and 1.30-1.73 (VAMP5, above), and fold change between day 0 and baseline −1.36-(−0.60) (GPA33, below), −0.82-(−0.39) (EPCAM, below), and −0.27-0.16 (CRNN, above). In some such embodiments, the recited baseline serum levels are measured by Olink. In some such embodiments, the subject has a greater likelihood of developing Grade 3+ CRS and/or Grade 3+ NE after immunotherapy than a subject having baseline serum levels that are below or above the median baseline serum levels, respectively. In such an embodiment, changes between baseline and Day 0 biomarker levels might be indicative of a systemic dysregulation in the subject as a result of any conditioning chemotherapy regimen, which in turn could predispose the subject to toxicity following administration of an immunotherapy (e.g. a CAR T-cell therapy).

The method above, wherein the combination therapy reduces cytokine induction after immunotherapy.

The method above, wherein the agent(s) is/are administered to the patient prior to CAR-T infusion, before the peak of CAR-T expansion (e.g., Day 0-6 post infusion), and/or at the peak CAR-T expansion (e.g., Day 7-14).

Any of the methods above, wherein the conditioning regimen/lymphodepleting therapy is selected from a different dose of cyclophosphamide/fludarabine than the subjects having the same or lower levels of the serum biomarkers recited in claim 6 (higher levels for baseline FGF2I and day 0 CGA, ICOSLG, ITM2A), bendamustine, total body irradiation, Anti-CD45 (Apamistamab), other chemotherapeutic agents (e.g. AVM0703, Busulfan, Thiotepa/Etoposide, Pentostatin), etc).

Any of the methods above, wherein the agent(s) is/are selected from anti-IL-1 (e.g. anakinra), T cell activation inhibitors (e.g. dasatinib), JAK inhibitors (e.g. filgotinib), anti-GM-CSF (e.g. lenzilumab), anti-TNF (e.g. infliximab), Ang2 inhibitors (e.g. azilsartan), anti-angiogenic therapies (e.g. bevacizumab), anti-IFNg (e.g. emapalumab-lzsg) etc.

A method of treating a cancer subject with low eosinophil and/or monocyte counts at day 0 (prior to) of immunotherapy by administering one or more agents or treatments that result in reduced inflammation. By way of non-limiting example, such agents include corticosteroids, anti-GMCSF, anti-IFNg, and also anti-IFN-α (Sifalimumab), and anti-IFN-b (Avonex).

The method above, wherein the subject has a low eosinophil and/or monocyte count when the day 0 eosinophil count is lower than (0.040, 0.065) and/or the day 0 monocyte count is lower than (0.025, 0.055).

A method of treating a cancer subject with a high tumor burden, wherein the immune activation mediated stress in the subject is reduced by administering one or more agents or treatments that result in a reduced inflammation (e.g., lower cytokine induction in the blood) and/or by using an alternative lymphodepleting regimen (relative to that of a subject with less than high tumor burden) prior to, during, and/or after immunotherapy.

The method above, wherein the reduced inflammation is favorable with respect to favorable for cell therapy.

The method above, wherein the subject has a high tumor burden (as assessed by SPD and/or tumor metabolic volume) when the baseline tumor burden (SPD) is greater than 2500, 3000, 3500, or 4000, preferably greater than 3000 mm² and/or the tumor metabolic volume is above the median for a representative tumor population (e.g., above 100, or above 150 ml).

A method of treating a cancer subject with a high international prognostic index (IPI), wherein the immune activation mediated stress in the subject is reduced by administering one or more agents or treatments that result in a reduced inflammation (e.g., lower cytokine induction in the blood) and/or by using an alternative lymphodepleting regimen (relative to that of a subject with less than high IPI) prior to, during, and/or after immunotherapy.

The method above, wherein the reduced inflammation is favorable with respect to favorable for cell therapy.

The methods above, wherein the subject has a high international prognostic index (IPI) when the IPI is greater than 1, preferably greater than 2, or more preferably greater than 3.

Any of the methods above, wherein the immunotherapy is CAR T cell therapy, TCR T cell therapy, tumor infiltrating lymphocytes (TIL) cell therapy, bispecific T-cell engagers (BiTEs), and/or administration of immune checkpoint inhibitors.

The method above, wherein the immune checkpoint inhibitor is selected from agents that block immune checkpoint receptors on the surface of T cells, such as cytotoxic T lymphocyte antigen 4 (CTLA-4), lymphocyte activation gene-3 (LAG-3), T-cell immunoglobulin mucin domain 3 (TIM-3), B- and T-lymphocyte attenuator (BTLA), T-cell immunoglobulin and T-cell immunoreceptor tyrosine-based inhibitory motif (ITIM) domain, and programmed cell death 1 (PD-1/PDL-1).

The method above, wherein the immunotherapy is autologous or allogeneic.

The method above, wherein the immunotherapy is CAR T or TCR T cell therapy that recognizes a target antigen.

The method above, wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C(TnC Al), thyroglobulin, tumor stromal antigens, vascular endothelial growth factor receptor-2 (VEGFR2), virus-specific surface antigen such as an HIV-specific antigen (such as HIV gp120), GPC3 (Glypican 3), as well as any derivate or variant of these antigens.

The method above, wherein the cancer/tumor is selected from a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

The method above, wherein the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

Any of the methods above, wherein the immunotherapy is selected from axicabtagene ciloleucel, brexucabtagene autoleucel, tisagenlecleucel, lisocabtagene maraleucel, and bb2121.

### BRIEF DESCRIPTION OF THE DRAWINGS

FIG. 1 is a schematic depicting adverse management differences between ZUMA-1 cohorts.

FIG. 2 shows a series of graphs depicting correlation between Olink NPX Expression and Internal MSD Data.

FIG. 3 is a chart depicting baseline 0-Link Analytes WGCNA analysis.

FIG. 4 shows plots of baseline protein analytes associated with Grade 3+ cytokine release syndrome (CRS) (left) or Grade 3+ neurologic events (NE) (right).

FIG. 5 is graph showing differentially expressed proteins at baseline in the yellow module which associate with responses to oxidative stress and metabolic process.

FIG. 6 is a graph showing differentially expressed proteins at baseline in the turquoise module which positively associate with leukocyte activation and adhesion.

FIG. 7 is a chart depicting day 0 0-Link WGCNA analysis.

FIG. 8 shows plots of day 0 protein analytes associated with Grade 3+ cytokine release syndrome (CRS) (left) or Grade 3+ neurologic events (NE) (right).

FIG. 9 is a graph showing differentially expressed proteins at day 0 in turquoise module which positively associate with adhesion.

FIG. 10. Is a graph showing differentially expressed proteins at day 0 in the blue module which associate with metabolic process.

FIG. 11 shows graphs of results of predictive models for Grade 3+ CRS and/or NE within 5 Days Post-CAR T infusion.

### DETAILED DESCRIPTION

The present disclosure is based in part on the discovery that pre- and post-conditioning proteomic characteristics of patients' serum may be associated with clinical efficacy and toxicity including durable responses, grade cytokine release syndrome, and grade 3 neurologic events.

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

### Insert Definition for Reference and Controls

The term “reference” describes a standard or control relative to which a comparison is performed. For example, in some embodiments, a protein level or a gene expression level of interest is compared with a reference or control that is a protein level or a gene expression level. In certain aspects, the reference value is from the same individual at an earlier time point. In some embodiments, a reference or control is tested, measured, and/or determined substantially simultaneously with the testing, measuring, or determination of interest. In some embodiments, a reference or control is a historical reference or control, optionally embodied in a tangible medium. In certain aspects, a reference or control is determined or characterized under comparable conditions, circumstances and/or within patient populations having common disease characteristics to those under assessment. In certain further aspects, sufficient similarities are present to justify reliance on and/or comparison to a selected reference or control.

As used throughout, the terms “baseline serum level” and “baseline (pre-conditioning) serum level” are used interchangeably and refer to a serum level of the one or more indicated biomarkers, where the serum sample was collected prior to administration of a pre-conditioning regimen to a cancer subject.

As used throughout, the terms “control baseline serum level” and “control baseline (pre-conditioning) serum level” are used interchangeably and refer to a historically observed baseline serum level of the one or more analytes or biomarkers disclosed, where the historically observed baseline serum level has not previously been observed to be associated with an onset of one or more adverse events, or is associated with a decreased risk of the onset of one or more adverse events. In some embodiments, deviations from the historically observed baseline serum level are correlated with increased rates of adverse events as a result of treatment with the immunotherapy.

As used throughout, the terms “day 0 serum level” and “day 0 (after conditioning and prior to immunotherapy)” are used interchangeably and refer to a serum level of the one or more indicated analytes or biomarkers, where the serum sample was collected following administration of a conditioning therapy to the cancer subject, but prior to administration of an immunotherapy to the cancer subject.

As used throughout, the terms “control day 0 serum level” and “control day 0 (after conditioning and prior to immunotherapy) serum level” are used interchangeably and refer to a historically observed day 0 serum level of the one or more analytes or biomarkers disclosed, where the historically observed day 0 serum level has not previously been observed to be associated with an onset of one or more adverse events, or is associated with a decreased risk of the onset of one or more adverse events. In some embodiments, deviations from the historically observed day 0 serum level are correlated with increased rates of adverse events as a result of treatment with the immunotherapy.

As used throughout, a “control level” or “control value” refers a historical value of a particular analyte observed in a population prior to administration of a cellular therapeutic product. In some embodiments, deviations from the historical value are correlated with adverse effects to the cellular therapy product. More specifically, in some embodiments, an increased level of an analyte in a test sample from a patient versus a control level for that corresponding analyte is associated with an increased chance of observing an adverse effect in the patient as a result of administration of the cellular therapeutic product. In such an embodiment, the increased chance of an adverse effect is measured with respect to a known and historical average likelihood of an adverse effect to the cellular therapeutic product in a population.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In one embodiment, the CAR T cell treatment comprises “axicabtagene ciloleucel treatment”. “Axicabtagene ciloleucel treatment” consists of a single infusion of anti-CD19 CAR transduced autologous T cells administered intravenously at a target dose of 2×106 anti-CD19 CART cells/kg. For subjects weighing greater than 100 kg, a maximum flat dose of 2×108 anti-CD19 CAR T cells may be administered. The anti-CD19 CAR T cells are autologous human T cells that have been engineered to express an extracellular single-chain variable fragment (scFv) with specificity for CD19 linked to an intracellular signaling part comprised of signaling domains from CD28 and CD3 (CD3-zeta) molecules arranged in tandem anti-CD19 CAR vector construct has been designed, optimized and initially tested at the Surgery Branch of the National Cancer Institute (NCI, IND 13871) (Kochenderfer et al, J Immunother. 2009; 32(7):689-702; Kochenderfer et al, Blood. 2010; 116(19):3875-86). The scFv is derived from the variable region of the anti-CD19 monoclonal antibody FMC63 (Nicholson et al, Molecular Immunology. 1997; 34(16-17):1157-65). A portion of the CD28 costimulatory molecule is added, as murine models suggest this is important for the anti-tumor effect and persistence of anti-CD19 CAR T cells (Kowolik et al, Cancer Res. 2006; 66(22):10995-1004). The signaling domain of the CD3-zeta chain is used for T cell activation. These fragments were cloned into the murine stem cell virus-based (MSGV1) vector, utilized to genetically engineer the autologous T cells. The CAR construct is inserted into the T cells' genome by retroviral vector transduction. Briefly, peripheral blood mononuclear cells (PBMCs) are obtained by leukapheresis and Ficoll separation. Peripheral blood mononuclear cells are activated by culturing with an anti-CD3 antibody in the presence of recombinant interleukin 2 (IL-2). Stimulated cells are transduced with a retroviral vector containing an anti-CD19 CAR gene and propagated in culture to generate sufficient engineered T cells for administration. Axicabtagene ciloleucel is a subject-specific product.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, [add other solid tumors] multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-C SF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, wherein the extracellular domain comprises a hinge domain, which may be truncated.

A “therapeutically effective amount,” “effective dose,” “effective amount,” or “therapeutically effective dosage” of a therapeutic agent, e.g., engineered CAR T cells, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g. animal model) systems.

The term “combination” refers to either a fixed combination in one dosage unit form, or a combined administration where a compound of the present disclosure and a combination partner (e.g. another drug as explained below, also referred to as “therapeutic agent” or “agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The terms “product” or “infusion product” are used interchangeably herein and refer to the T cell composition that is administered to the subject in need thereof. Typically, in CAR T-cell therapy, the T cell composition is administered as an infusion product.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2R13, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

In the context of this disclosure, the term “TN,” “T naïve-like”, and CCR7+CD45RA+ actually refers to cells that are more like stem-like memory cells than like canonical naïve T cells. Accordingly, all references in the Examples and Claims to TN refers to cells that were experimentally selected only by their characterization as CCR7+CD45RA+ cells and should be interpreted as such. Their better name in the context of this disclosure is stem-like memory cells, but they shall be referred to as CCR7+CD45RA+ cells. Further characterization into stem-like memory cells may be done for example using the methods described in Arihara Y, Jacobsen C A, Armand P, et al. Journal for ImmunoTherapy of Cancer. 2019; 7(1):P210.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” or a “subject” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In some embodiments, the treatment may be prophylactic, in which case the treatment is administered before any symptoms of the condition are observed. The term “prophylaxis” as used herein means the prevention of or protective treatment for a disease or disease state. Prevention of a symptom, disease, or disease state may include reduction (e.g., mitigation) of one or more symptoms of the disease or disease state, e.g., relative to a reference level (e.g., the symptom(s) in a similar subject not administered the treatment). Prevention may also include delaying onset of one or more symptoms of the disease or disease state, e.g., relative to a reference level (e.g., the onset of the symptom(s) in a similar subject not administered the treatment). In embodiments, a disease is a disease described herein. In some embodiments, the disease is cancer. In some embodiments, the diseased state is CRS or neurotoxicity. In some embodiments, indicators of improvement or successful treatment include determination of the failure to manifest a relevant score on toxicity grading scale (e.g. CRS or neurotoxicity grading scale), such as a score of less than 3, or a change in grading or severity on the grading scale as discussed herein, such as a change from a score of 4 to a score of 3, or a change from a score of 4 to a score of 2, 1 or 0.

As used herein, “myeloid cells” are a subgroup of leukocytes that includes granulocytes, monocytes, macrophages, and dendritic cells.

In one embodiment, the terms “high” and “low” mean “above” and “below” the median value for a representative population of subjects. In one embodiment, the terms mean in the upper or lower quartiles, respectively. Both the mean and the quartile distribution may be determined by one of ordinary skill in the art by routine methods.

As used herein, the term “quartile” is a statistical term describing a division of observations into four defined intervals based upon the values of the data and how they compare to the entire set of observations.

As used herein, the term “Study day 0” is defined as the day the subject received the first CAR T cell infusion. The day prior to study day 0 will be study day −1. Any days after enrollment and prior to study day −1 will be sequential and negative integer-valued.

As used herein, the term “durable response” refers to the subjects who were in ongoing response at least by one year follow up post CAR T cell infusion. In one embodiment, “duration of response” is defined as the time from the first objective response to disease progression or to death due to disease relapse.

As used herein, the term “relapse” refers to the subjects who achieved a complete response (CR) or partial response (PR) and subsequently experienced disease progression.

As used herein, the term “non-response” refers to the subjects who had never experienced CR or PR post CAR T cell infusion, including subjects that with stable disease (SD) and progressive disease (PD).

As used herein, the term “objective response” refers to complete response (CR), partial response (PR), or non-response. It may be assessed per revised IWG Response Criteria for Malignant Lymphoma (Cheson et al., J Clin Oncol. 2007; 25(5):579-86)

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution.

As used herein “objective response rate” (ORR) is determine per International Working Group (IWG) 2007 criteria (Cheson et al. J Clin Oncol. 2007; 25(5):579-86).

As used herein “progression-free survival (PFS)” may be defined as the time from the T cell infusion date to the date of disease progression or death from any cause. Progression is defined per investigator's assessment of response as defined by IWG criteria (Cheson et al., J Clin Oncol. 2007; 25(5):579-86).

The term “overall survival (OS)” may be defined as the time from the T cell infusion date to the date of death from any cause.

As used herein, the expansion and persistence of CART cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+PBMC/μL in serum attained after Day 0.

As used herein, the “time to Peak of CART cell” is defined as the number of days from Day 0 to the day when the peak of CART cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Baseline}{Baseline}$$

As used herein, the “peak of cytokine post baseline” is defined as the maximum level of cytokine in serum attained after baseline (Day −5) up to Day 28.

As used herein, the “time to peak of cytokine” post CAR T cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

As used herein, treatment-emergent adverse events (TEAEs) are defined as adverse events (AE) with onset on or after the first dose of conditioning chemotherapy. Adverse events may be coded with the Medical Dictionary for Regulatory Activities (MedDRA) version 22.0 and graded using the National Cancer Institute (NCI) Common Terminology Criteria for Adverse Events (CTCAE) version 4.03. Cytokine Release Syndrome (CRS) events may be graded on the syndrome level per Lee and colleagues (Lee et al, 2014 Blood. 2014; 124(2):188-95. Individual CRS symptoms may be graded per CTCAE 4.03. Neurologic events may be identified with a search strategy based on known neurologic toxicities associated with CAR T immunotherapy, as described in, for example, Topp, M S et al. Lancet Oncology. 2015; 16(1):57-66.

Various aspects of the disclosure are described in further detail in the following subsections.

### Characterization of the Serum Protein Profile of Immunotherapy Cancer Patients

In some embodiments, the present disclosure provides methods to characterize the serum proteomic profile of a cancer patient prior to treatment with immunotherapy and/or pre-conditioning. In one embodiment, immunotherapy is selected from treatment with a chimeric receptor therapy (e.g., YESCARTA™ axicabtagene ciloleucel (axi-cel), TECARTUS™-brexucabtagene autoleucel/KTE-X19, KYIVIRIAH™ (tisagenlecleucel), etc), TCR, TIL, immune check point inhibitors, among others. In one embodiment, the immunotherapy product comprises autologous or allogeneic CAR T cells. In one embodiment, the immunotherapy comprises T-Cell Receptor-modified T cells. In one embodiment, the immunotherapy comprises tumor infiltrating lymphocytes (TILs). In one embodiment, the immunotherapy product comprises Induced Pluripotent Stem Cells (iPSCs). As described herein, in some embodiments, the serum protein characteristics are obtained through pre-specified protein sets and analyzed through OPI and machine learning models. In some embodiments, the serum levels may be measured by ELISA. In some embodiments, the serum protein profiles associate with adverse events of chimeric receptor therapy (e.g., axicabtagene ciloleucel (axi-cel)) and may be used to predict adverse events in response to all immunotherapies (e.g., T cells, non-T cells, TCR-based therapies, CAR-based therapies, bispecific T-cell engagers (BiTEs), and/or immune checkpoint blockade).

In one embodiment, the disclosure provides that baseline (pre-conditioning) serum levels of certain protein associated with metabolic processes and leukocyte activation correlate positively with, and can be biomarkers for, poor prognosis factors for immunotherapy including international prognostic index and baseline tumor burden. In one embodiment, the immunotherapy is T cell therapy. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells In one embodiment, the immunotherapy is CAR T cell or TCR T cell therapy. In one embodiment, the immunotherapy is anti-CD19 CART cell therapy.

Accordingly, in one embodiment, the disclosure provides a method of predicting international prognostic index and baseline tumor burden parameters in a cancer patient based on the baseline (pre-conditioning) serum levels of metabolic process markers and/or leukocyte activation markers in the patient.

In one embodiment, the disclosure provides that baseline (pre-conditioning) serum levels of certain proteins associated with IL1/IL6 pathway correlate positively with, and can be biomarkers for, Grade 3+ cytokine release syndrome (CRS) after immunotherapy. In one embodiment, the proteins associated with the IL1/IL6 pathway are selected from IL1A and OSMR. Accordingly, in one embodiment, the disclosure provides a method of predicting the likelihood that a patient will develop Grade 3+ CRS after immunotherapy based on measurements of baseline serum levels of IL1A and OSMR, wherein the higher the serum level of one or more of IL1A and OSMR the greater the chance that the patient will develop Grade 3+ CRS after immunotherapy. In one embodiment, this information is utilized to make decisions related to the immunotherapy including whether or not to administer immunotherapy, what dosage of immunotherapy to administer, what dosage regimen to follow, and/or what agents should be administered to the patient prior to, after, and/or during immunotherapy administration to improve management and/or reduce Grade 3+ CRS in the patient.

In one embodiment, the disclosure provides that baseline (pre-conditioning) serum levels of certain proteins associated with inflammatory endothelial markers correlate positively with, and can be biomarkers for, both Grade 3+ CRS and Grade 3+ neurologic events (NE) after immunotherapy. In one embodiment, the inflammatory endothelial markers are ACE2, CEACAM1, ICAM2, and ADAM15. In one embodiment, the disclosure provides that baseline serum levels of MET, OSMR, ACE2, ACY1, and BILDIR correlate positively with, and can be biomarkers for, Grade 3+ CRS and NE after immunotherapy. Accordingly, in one embodiment, the disclosure provides a method of predicting the likelihood that a patient will develop Grade 3+CRS and/or Grade 3+ NE after immunotherapy based on measurements of baseline serum levels of ACE2, CEACAM1, ICAM2, ADAM15, MET, OSMR, ACY1, and BILDIR, wherein the higher the serum level of one or more of ACE2, CEACAM1, ICAM2, ADAM15, MET, OSMR, ACE2, ACY1, BILDIR the greater the chance that the patient will develop Grade 3+ CRS and/or Grade 3+ NE after immunotherapy. In one embodiment, this information is utilized to make decisions related to the immunotherapy including whether or not to administer immunotherapy, what dosage of immunotherapy to administer, what dosage regimen to follow, and/or what agents should be administered to the patient prior to, after, and/or during immunotherapy administration to improve management and/or reduce Grade 3+ CRS and/or Grade 3+ NE in the patient.

In one embodiment, the disclosure provides that day 0 (i.e., immediately prior to immunotherapy and after conditioning therapy) baseline serum levels of CCL16, CELA3A, MEGF9, MFAP3, and REG1B and day 0 serum levels of AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ITM2A, ICOSLG, CGA, CRNN, LY96, AST, and VAMP5 correlate positively with, and can be biomarkers for, Grade 3+ CRS and Grade 3+ NE after immunotherapy. Accordingly, in one embodiment, the disclosure provides a method of predicting the likelihood that a patient will develop Grade 3+CRS and/or Grade 3+ NE after immunotherapy based on measurements of day 0 serum levels of one or more of CCL16, CELA3A, MEGF9, MFAP3, and REG1B and/or one or more of AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ITM2A, ICOSLG, CGA, CRNN, LY96, AST, and VAMP5, wherein the higher the day 0 serum level of one or more of CCL16, CELA3A, MEGF9, MFAP3, and REG1B and/or one or more of AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ITM2A, ICOSLG, CGA, CRNN, LY96, AST, and VAMP5, the greater the chance that the patient will develop Grade 3+ CRS and/or Grade 3+ NE after immunotherapy. In one embodiment, this information is utilized to make decisions related to the immunotherapy including whether or not to administer immunotherapy, what dosage of immunotherapy to administer, what dosage regimen to follow, and/or what agents should be administered to the patient prior to, after, and/or during immunotherapy administration to improve management and/or reduce Grade 3+ CRS and/or Grade 3+ NE in the patient.

In one embodiment, the disclosure provides that the value for the fold change between day 0 and baseline serum levels of GPA33, EPCAM, and CRNN correlate positively with, and can be biomarkers for, Grade 3+ CRS and NE after immunotherapy. In one embodiment, serum levels of one or more of these proteins correlate positively with, and can be biomarkers for, immune activation induced stress after immunotherapy; such markers can be downregulated after lymphodepletion. In one embodiment, the conditioning therapy comprises cyclophosphamide and fludarabine. Accordingly, in one embodiment, the disclosure provides a method of predicting the likelihood that a patient will develop Grade 3+ CRS and/or Grade 3+ NE after immunotherapy based on the ratio/fold change between day 0 and baseline measurements of serum levels of one or more of GPA33, EPCAM, and CRNN, wherein the higher the ratio between day 0 and baseline serum level of one or more of GPA33, EPCAM, and CRNN, the greater the chance that the patient will develop Grade 3+ CRS and/or Grade 3+ NE after immunotherapy. In one embodiment, this information is utilized to make decisions related to the immunotherapy including whether or not to administer immunotherapy, what dosage of immunotherapy to administer, what dosage regimen to follow, and/or what agents should be administered to the patient prior to, after, and/or during immunotherapy administration to improve management and/or reduce Grade 3+ CRS and/or Grade 3+ NE in the patient. Also accordingly, in one embodiment, the disclosure provides a method of predicting immune activation induced stress after immunotherapy based on the ratio between day 0 and baseline measurements of serum levels of one or more of GPA33, EPCAM, and CRNN, wherein the higher the ration between day 0 and baseline serum level of one or more of GPA33, EPCAM, and CRNN, the greater the immune activation induced stress after immunotherapy. In one embodiment, this information is utilized to make decisions related to the immunotherapy including whether or not to administer immunotherapy, what dosage of immunotherapy to administer, what dosage regimen to follow, and/or what agents should be administered to the patient prior to, after, and/or during immunotherapy administration to reduce immune activation induced stress after immunotherapy.

Also accordingly, the disclosure provides a method to stratify patients having greater likelihood of developing high grade adverse events (e.g., Grade 3+ CRS and/or Grade 3+ NE), the method comprising administering immunotherapy in combination with an agent(s) that reduces immune activation and/or endothelial cells disruption, wherein the combination therapy reduces cytokine induction and/or wherein the combination therapy reduces the endothelial cell disruption, wherein the patient is selected for combination therapy when the patient has high level of serum markers, estimated by measuring (i) the protein level of one or more of baseline MET, OSMR, ACE2, ACY1, FGF21, BILDIR; day 0 CCL16, CELA3A, MEGF9, MFAP3, REG1B, AREG, BSG CKAP4, CXCL1, EIF5A, IL1A, KIFBP, KIRREL2, NUB1, OSMR, PAG1, PCDH17, STK11, ACE2, ADAM15, CEACAM1, HLA-DRA, ICAM2, LY9, REG3A, SERPINB9, SOD2, ADA2, ITM2A, ICOSLG, CGA, CRNN, LY96, AST, and VAMP5; and (ii) the fold change between day 0 and baseline GPA33, EPCAM, and CRNN in the blood. In one embodiment, a high level of serum biomarkers is a level that is at least 1.5 times greater than a reference level.

In one embodiment, the serum levels are considered high and the subject is predicted to have a higher likelihood of Grade 3+ CRS and/or Grade 3+ NE and/or be elected for combination therapy, when the serum levels are above/below (as underlined) the following values for each of the respective proteins: baseline 0.56-0.75 (MET, above), 0.62-0.77 (OSMR, above), 0.65-0.98 (ACE2, above), 0.66-1.10 (ACY1, above), 1.58-2.25 (FGF21, below), 4.12-5.50 (BILDIR, above), day 0 0.84-1.25 (CCL16, above), 1.37-2.01 (CELA3A, above), 0.26-0.48 (MEGF9, above), 0.33-0.49 (MFAP3, above), 1.49-1.99 (REG1B, above), 0.88-1.22 (AREG, above), 0.73-0.96 (BSG, above) 1.83-2.31 (CKAP4, above), 3.48-3.91 (CXCL1, above), 0.09-0.23 (EIF5A, above), 0.46-0.81 (IL1A, above), 1.36-1.66 (KIFBP, above), 1.62-2.13 (KIRREL2, above), 1.39-1.77 (NUB1, above), 0.65-0.79 (OSMR, above), 2.16-2.74 (PAG1, above), 1.03-1.32 (PCDH17, above), 1.09-1.35 (STK11, above), 1.20-1.65 (ACE2, above), 1.09-1.31 (ADAM15, above), 0.69-0.90 (CEACAM1, above), 1.13-1.41 (HLA-DRA, above), 1.21-1.43 (ICAM2, above), 0.11-0.49 (LY9, above), 2.34-2.88 (REG3A, above), 2.12-2.30 (SERPINB9, above), 1.37-1.86 (SOD2, above), 0.70-0.98 (ADA2, above), 0.74-1.01 (ITM2A, below), 0.45-0.56 (ICOSLG, below), 1.12-1.50 (CGA, above), −0.48-0.08 (CRNN, above), 0.54-0.70 (LY96, above), 42.12-65.80 (AST, above), and 1.30-1.73 (VAMP5, above), and fold change between day 0 and baseline −1.36-(−0.60) (GPA33, below), −0.82-(−0.39) (EPCAM, below), and −0.27-0.16 (CRNN, above);

In one embodiment, a high level of serum biomarkers is a level at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, or at least 100 fold over median. In one embodiment, the fold change between day 0 and baseline GPA33, EPCAM, and CRNN in the blood that is a trigger for combination therapy is at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, or at least 100 fold. In one embodiment, the plasma levels of the protein biomarkers are high when they fall within the second, third, or fourth quartile of levels among those in a representative tumor population, as assessed by one of ordinary skill in the art. In one embodiment, the levels of the protein biomarkers are high or low, respectively, when they fall 0-0.1%, 0.1%-0.5%, 0.5%-1.0%, 1-5%, 5-10%, 10-15%, 15-20%, 20-25%, 25-30%, 30-35%, 35-40%, 40-45%, 45-50%, 50-55%, 55-60%, 60-65%, 65-70%, etc. 95%-100%) above or below, respectively, the median or the values specified above. All listed values may be modified by the term “above.”

In one embodiment, the disclosure provides a method of treating a subject with a low eosinophil and/or monocyte counts at day 0 by administering one or more agents or treatments that result in reduced inflammation.

In one embodiment, the disclosure provides a method of treating a subject with immunotherapy having a high tumor burden, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high tumor burden (as assessed by SPD and/or tumor metabolic volume) when the baseline tumor burden (SPD) is greater than 2500, 3000, 3500, or 4000, preferably greater than 3000 mm² and/or the tumor metabolic volume is above the median for a representative tumor population (e.g., above 100, or above 150 ml).

In one embodiment, the disclosure provides a method of treating a subject with a high international prognostic index, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high international prognostic index (IPI) when the IPI is greater than 1, 2 or 3.

In one embodiment, the immunotherapy is T cell therapy. In one embodiment, the T cell therapy is autologous. In one embodiment, the T cell therapy is allogeneic. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), iPSCs, checkpoint inhibitors, and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells. In one embodiment, the immunotherapy is CAR T cell or TCR T cell therapy. In one embodiment, the immunotherapy is anti-CD19 CART cell therapy. Examples of target tumor antigens are listed elsewhere in the specification. Examples of cancers that may be treated by the methods of the disclosure are also provided elsewhere in the specification.

In one embodiment, the agent(s) that is administered in combination with immunotherapy and reduces immune activation and/or endothelial cells disruption, wherein the combination therapy reduces cytokine induction and/or wherein the combination therapy reduces the endothelial cell disruption, is/are selected from anti-IL-1 (e.g. anakinra), T cell activation inhibitors (e.g. dasatinib), JAK inhibitors (e.g. filgotinib), anti-GM-CSF (e.g. lenzilumab), anti-TNF (e.g. infliximab), Ang2 inhibitors (e.g. azilsartan), anti-angiogenic therapies (e.g. bevacizumab), anti-IFNg (e.g. emapalumab-lzsg) etc. In one embodiment, the immunotherapy is administered in a combination therapy that enhances the proliferation of the T cells. In one embodiment, said combination therapy comprises treatment with pembrolizumab, lenalidomide, epcoritamab, and utoliumab. In one embodiment, said therapy comprises magrolimab (anti-CD47 antagonist), GSK3745417 (STING agonist), INCB001158 (ARG1/2 inhibitor), GS-1423 (CD73×TGFβ mAb), Selicrelumab (CD40 agonist), GS3583 (FLT3 agonist), Pexidartinib (CSF1R inhibitor, epacadostat (IDO1 inhibitor), GS9620 (TLR agonist). In one embodiment, the agent is selected from (i) a GM-CSF inhibitor selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule; (ii) a CSF1 inhibitor selected from RG7155, PD-0360324, MCS110/lacnotuzumab), or a biosimilar version of any one of the same; and a small molecule; and/or (iii) a GM-CSFR inhibitor and the CSF1R inhibitor selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or R05509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule. In some embodiments, additional treatments may be cytokines (e.g., IL-2, IL-15), stimulating antibodies (e.g., anti-41BB, OX-40), checkpoint blockade (e.g., CTLA4, PD-1), or innate immune stimulators (e.g., TLR, STING agonists). In some embodiments, additional treatments may be T cell-recruiting chemokines (e.g., CCL2, CCL1, CCL22, CCL17, and combinations thereof). In some embodiments, the additional therapy or therapies are administered systemically or intratumorally. In some embodiments, the additional therapy that is used in combination is administered together with conditioning and/or immunotherapy. In some embodiments, the additional therapy that is used in combination is administered sequentially with conditioning and/or immunotherapy.

In one embodiment, the agents may/should be administered to the patient prior to, after, and/or during immunotherapy administration to reduce Grade 3+ CRS in the subject. In one embodiment, the agent(s) is/are administered to the patient prior to CAR-T infusion, before the peak of CAR-T expansion (e.g., Day 0-6 post infusion), and/or at the peak CAR-T expansion (e.g., Day 7-14). In one embodiment, the peak of CAR-T expansion is Day 7-14 post infusion. In one embodiment, the peak of CAR-T expansion is Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, or Day 20 post-infusion. In one embodiment, the period after peak CAR-T expansion is the period between Day 14-28 post-infusion. In one embodiment, the period after peak CAR-T expansion is Day 1-Day 5, Day 5-Day 10, Day 10-Day 15, Day 15-Day 20, Day 20-Day 25; after Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 25, Day 30, Day 35, Day 40, Day 45, Day 50, any day after peak expansion.

In one embodiment, the immunotherapy is combined with low dose radiation, promotion of T cell activity through immune checkpoint blockade, and/or T cell agonists. In one embodiment, the T cell agonist is selected from pembrolizumab, lenalidomide, epcoritamab, and utoliumab. In one embodiment, the combination agent is selected from check-point inhibitors (e.g., anti-PD1 antibodies, pembrolizumab (Keytruda), Cemiplimab (Libtayo), nivolumab (Opdivo); anti-PD-L1 antibodies, Atezolizumab (Tecentriq), Avelumab (Bavencio), Durvalumab (Imfinzi); and/or anti-CTLA-4 antibodies, Ipilimumab (Yervoy)).

In one embodiment, the pre-conditioning regimen is a lymphodepleting regimen. In one embodiment, the lymphodepletion therapy regimen(s) is/are selected from one of several possible regimens of cyclophosphamide/fludarabine, bendamustine, total body irradiation, Anti-CD45 (Apamistamab), and other chemotherapeutic agents (e.g. AVM0703, Busulfan, Thiotepa/Etoposide, Pentostatin). Additional conditioning methods and regimens can be found elsewhere in the specification.

In one embodiment, the disclosure provides a method of improving immunotherapy (e.g. CAR T cell treatment) by optimization of bridging therapy to modulate the tumor microenvironment to a more favorable immune permissive state. In one embodiment, the optimization comprises administering bridging therapy with Immunomodulatory imide drugs (IMIDs)/cereblon modulators (e.g., lenoalidomide, pomalidomide, iberdomide, and apremilast). In one embodiment, the optimization comprises administering bridging therapy with local radiation.

In one embodiment, the disclosure provides a method of improving immunotherapy (e.g. CAR T cell treatment) by optimization of bridging therapy to diminish tumor burden prior immunotherapy (e.g. CAR T cell treatment) administration. In one embodiment, the optimization comprises administering bridging therapy with R-CHOP, bendamustine, alkylating agents, and/or platinum-based agents. Other exemplary bridging therapies are described elsewhere in this application.

In one embodiment, the disclosure provides a method of improving immunotherapy (e.g. CAR T cell treatment) by optimization of conditioning treatment to modulate the tumor microenvironment to a more favorable immune permissive state (e.g., less myeloid inflammation in the TME). In one embodiment, the optimization comprises addition of local irradiation to cyclophosphamide/fludarabine conditioning. In one embodiment, the optimization comprises administration of platinum-based agents as conditioning agents.

In one embodiment, the disclosure provides a method of improving immunotherapy (e.g. CAR T cell treatment) by coadministration of biological response modifiers together or post-immunotherapy (e.g. CAR T cell treatment) administration to enable CAR T cell activity. In one embodiment, the method comprises administration of gamma chain cytokines (e.g., IL-2, IL-4, IL-7, IL-9, IL-15, and IL-21). In one embodiment, the method comprises administration of checkpoint blocking agents (e.g. anti-CTLA-4).

In one embodiment, the disclosure provides a method of improving immunotherapy (e.g. CAR T cell treatment) by reprogramming of T cells to overcome detrimental tumor microenvironments, including low T/M ratio, high tumor burden, high TME myeloid cell density and/or high TME myeloid inflammation levels. In one embodiment, the T cells are engineered to express gamma chain receptor cytokines. In one embodiment, the gamma chain receptor cytokines are expressed under constitutive or inducible promoters.

In one embodiment, the disclosure provides a method of improving CAR T cell treatment by optimizing T cell manufacturing to help CAR T cells overcome detrimental tumor microenvironments, wherein the characteristics of the tumor microenvironment that may be detrimental comprise low T/M ratio, high tumor burden, high TME myeloid cell density and/or high TME myeloid inflammation levels. In one embodiment, the characteristics of the TME that may be detrimental comprise low T/M ratio (within −0.5-4), high tumor burden (within 3000-40000 mm²), high myeloid cell density (within 1000-4000 cells/mm²) and/or high TME myeloid inflammation levels (within 27-2000). In one embodiment, the method comprises engineering CAR T cells to express gamma chain receptor cytokines. In one embodiment, the gamma chain receptor cytokines are expressed under constitutive or inducible promoters. In one embodiment, the method comprises growing the T cells in the presence of gamma chain cytokines such as IL-15.

### Clinical Outcomes

In some embodiments, the clinical outcome is complete response. In some embodiments, the clinical outcome is durable response. In some embodiments, the clinical outcome is complete response. In some embodiments, the clinical outcome is no response. In some embodiments, the clinical outcome is partial response. In some embodiments, the clinical outcome is objective response. In some embodiments, the clinical outcome is survival. In some embodiments, the clinical outcome is relapse.

In some embodiments, objective response (OR) is determined per the revised IWG Response Criteria for Malignant Lymphoma (Cheson, 2007) and determined by IWG Response Criteria for Malignant Lymphoma (Cheson et al. Journal of Clinical Oncology 32, no. 27 (September 2014) 3059-3067). Duration of Response is assessed. The Progression-Free Survival (PFS) by investigator assessment per Lugano Response Classification Criteria is evaluated.

In some embodiments, part of the clinical outcome is the evaluation of adverse events. In this regard, CRS grading was done according to Lee D W et al., (2014). Current concepts in the diagnosis and management of cytokine release syndrome. Blood. 2014 Jul. 10; 124(2): 188-195. Neurologic toxicity was assessed by monitoring patients for signs and symptoms of neurologic toxicities by ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the method comprises monitoring patients at least daily for 7 days at the certified healthcare facility following infusion for signs and symptoms of neurologic toxicities. In some embodiments, the method comprises monitoring patients for signs or symptoms of neurologic toxicities for 4 weeks after infusion.

In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

Clinical outcomes of CAR T cell treatment are dependent on the level of CAR T cells in the blood. In some embodiments, response, levels of CAR T cells in blood, or immune related factors is determined by follow up at about 1 day, about 2 days, about 3 days, about 4 days, about 5 days, about 6 days, or about 7 days after administration of engineered CAR T cells. In some embodiments, response, levels of CAR T cells in blood, or immune related factors is determined by follow up at about 1 week, about 2 weeks, about 3 weeks, or about 4 weeks after administration of engineered CART cells. In some embodiments, response, levels of CART cells in blood and/or immune related factors are determined by follow up at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administration of a engineered CAR T cells. In some embodiments, response, levels of CAR T cells in blood and/or immune related factors are determined by follow up at about 1 year, about 1.5 years, about 2 years, about 2.5 years, about 3 years, about 4 years, or about 5 years after administration of engineered CAR T cells.

In some embodiments, methods described herein may provide a clinical benefit to a subject. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, approximately 1%, 2%, 3%, 4%, 5%, 6%, 7%, 8%, 9%, 10%, 15%, 20%, 25%, 30%, 35%, 40%, 45%, 0%, 55%, 60%, 65%, 70%, 75%, 80%, 85%, 90% or 95% and any unenumerated % in between of patients achieve a clinical benefit. In some embodiments, the response rate is 1%, 2%, 3%, 4%, 5%, 6%, 7%, 8%, 9%, 9.5%, 10.5%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 25 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81, 82, 83, 84, 85, 86, 87, 88, 89, 90, 91, 92, 93, 94, 95, 96, 97, 98, 99, or 100% or some other unenumerated percentage and range in between 1% and 100%. In some embodiments, the response rate is between 0%-10%, 10%-20%, 20%-30%, 30%-40%, 40%-50%, 50%-60%, 60%-70%, 70%-80%, 80%-90%, or 90%-100%. In some embodiments, the response rate is between 0%-1.%, 1%-1.5%, 1.5%-2%, 2%-3%, 3%-4%, 4%-5%, 5%-6%, 6%-7%, 7%-8%, 8%-9%, 9%-10%, 10%-15%, 15%-20%, 20-25%, 25%-30%, 35-40%, and so one and so forth, through 95%-100%.

### Chimeric Antigen Receptors

In one embodiment, the immunotherapy is CAR-T cell immunotherapy. Chimeric antigen receptors (CARs) are genetically engineered receptors. These engineered receptors may be inserted into and expressed by immune cells, including T cells and other lymphocytes in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016).

In some embodiments, a costimulatory domain which includes a truncated hinge domain (“THD”) further comprises some or all of a member of the immunoglobulin family such as IgG1, IgG2, IgG3, IgG4, IgA, IgD, IgE, IgM, or fragment thereof.

In some embodiments, the THD is derived from a human complete hinge domain (“CHD”). In other embodiments, the THD is derived from a rodent, murine, or primate (e.g., non-human primate) CHD of a costimulatory protein. In some embodiments, the THD is derived from a chimeric CHD of a costimulatory protein.

The costimulatory domain for the CAR of the disclosure may further comprise a transmembrane domain and/or an intracellular signaling domain. The transmembrane domain may be fused to the extracellular domain of the CAR. The costimulatory domain may similarly be fused to the intracellular domain of the CAR. In some embodiments, the transmembrane domain that naturally is associated with one of the domains in a CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this disclosure may be derived from (i.e., comprise) 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CD S, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Optionally, short linkers may form linkages between any or some of the extracellular, transmembrane, and intracellular domains of the CAR. Examples of such short linkers are described in WO 2019/060695, which is incorporated by reference in its entirety.

The linkers described herein, may also be used as a peptide tag. The linker peptide sequence may be of any appropriate length to connect one or more proteins of interest and is preferably designed to be sufficiently flexible so as to allow the proper folding and/or function and/or activity of one or both of the peptides it connects. Thus, the linker peptide may have a length of no more than 10, no more than 11, no more than 12, no more than 13, no more than 14, no more than 15, no more than 16, no more than 17, no more than 18, no more than 19, or no more than 20 amino acids. In some embodiments, the linker peptide comprises a length of at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the linker comprises at least 7 and no more than 20 amino acids, at least 7 and no more than 19 amino acids, at least 7 and no more than 18 amino acids, at least 7 and no more than 17 amino acids, at least 7 and no more than 16 amino acids, at least 7 and no more 15 amino acids, at least 7 and no more than 14 amino acids, at least 7 and no more than 13 amino acids, at least 7 and no more than 12 amino acids or at least 7 and no more than 11 amino acids. In certain embodiments, the linker comprises 15-17 amino acids, and in particular embodiments, comprises 16 amino acids. In some embodiments, the linker comprises 10-20 amino acids. In some embodiments, the linker comprises 14-19 amino acids. In some embodiments, the linker comprises 15-17 amino acids. In some embodiments, the linker comprises 15-16 amino acids. In some embodiments, the linker comprises 16 amino acids. In some embodiments, the linker comprises 3, 4, 5, 6, 7, 8, 9, 10, 11, 12, 13, 14, 15, 16, 17, 18, 19 or 20 amino acids.

In some embodiments, a spacer domain is used. In some embodiments, the spacer domain is derived from CD4, CD8a, CD8b, CD28, CD28T, 4-1BB, or other molecule described herein. In some embodiments, the spacer domains may include a chemically induced dimerizer to control expression upon addition of a small molecule. In some embodiments, a spacer is not used.

The intracellular (signaling) domain of the engineered T cells of the disclosure may provide signaling to an activating domain, which then activates at least one of the normal effector functions of the immune cell. Effector function of a T cell, for example, may be cytolytic activity or helper activity including the secretion of cytokines.

In certain embodiments, suitable intracellular signaling domain include (i.e., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Ly108), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

### Antigen Binding Molecules

Suitable CARs and TCRs may bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (“scFv”). A scFv is a single chain antibody fragment having the variable regions of the heavy and light chains of an antibody linked together. See U.S. Pat. Nos. 7,741,465 and 6,319,494, as well as Eshhar et al., Cancer Immunol Immunotherapy (1997) 45: 131-136. A scFv retains the parent antibody's ability to interact specifically with target antigen. scFv's are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR or TCR such that it is capable of recognizing and binding to the antigen of interest. Bispecific and multispecific CARs and TCRs are contemplated within the scope of the disclosure, with specificity to more than one target of interest.

In some embodiments, the polynucleotide encodes a CAR or TCR comprising a (truncated) hinge domain and an antigen binding molecule that specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-AL major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface antigens.

### Engineered T Cells and Products

In one embodiment, the immunotherapy is T cell therapy. In some embodiments, the donor T cells for use in the T cell therapy are obtained from the patient (e.g., for an autologous T cell therapy). In other embodiments, the donor T cells for use in the T cell therapy are obtained from a subject that is not the patient. In certain embodiments, the T cell is a tumor-infiltrating lymphocyte (TIL), engineered autologous T cell (eACT™), an allogeneic T cell, a heterologous T cell, or any combination thereof. In some embodiments, the T cells are obtained from a donor subject. In some embodiments, the donor subject is human patient afflicted with a cancer or a tumor. In some embodiments, the donor subject is a human patient not afflicted with a cancer or a tumor.

In one embodiment, the cells are obtained from a subject. In one embodiment, the cells are Induced Pluripotent Stem Cells (iPSCs). T cells may be obtained from, e.g., peripheral blood mononuclear cells, bone marrow, lymph node tissue, cord blood, thymus tissue, tissue from a site of infection, ascites, pleural effusion, spleen tissue, tumors, or differentiated in vitro. In addition, the T cells may be derived from one or more T cell lines available in the art. T cells may also be obtained from a unit of blood collected from a subject using any number of techniques known to the skilled artisan, such as FICOLL™ separation and/or apheresis. In some embodiments, the cells collected by apheresis are washed to remove the plasma fraction, and placed in an appropriate buffer or media for subsequent processing. In some embodiments, the cells are washed with PBS. As will be appreciated, a washing step may be used, such as by using a semi-automated flow through centrifuge, e.g., the Cobe™ 2991 cell processor, the Baxter CytoMate™, or the like. In some embodiments, the washed cells are resuspended in one or more biocompatible buffers, or other saline solution with or without buffer. In some embodiments, the undesired components of the apheresis sample are removed. Additional methods of isolating T cells for a T cell therapy are disclosed in U.S. Patent Pub. No. 2013/0287748, which is herein incorporated by references in its entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8+ cells are further sorted into naive, central memory, and effector cells by identifying cell surface antigens that are associated with each of these types of CD8+ cells. In some embodiments, the expression of phenotypic markers of central memory T cells includes expression of CCR7, CD3, CD28, CD45RO, CD62L, and CD127 and negative for granzyme B. In some embodiments, central memory T cells are CD8+, CD45RO+, and CD62L+ T cells. In some embodiments, effector T cells are negative for CCR7, CD28, CD62L, and CD127 and positive for granzyme B and perforin. In some embodiments, CD4+ T cells are further sorted into subpopulations. For example, CD4+ T helper cells may be sorted into naive, central memory, and effector cells by identifying cell populations that have cell surface antigens.

In some embodiments, the immune cells, e.g., T cells, are genetically modified (engineered) following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, a composition comprising engineered T cells comprises a pharmaceutically acceptable carrier, diluent, solubilizer, emulsifier, preservative and/or adjuvant. In some embodiments, the composition comprises an excipient. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the composition is selected for parenteral delivery, for inhalation, or for delivery through the digestive tract, such as orally. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In some embodiments, the vehicle for parenteral injection is sterile distilled water in which composition described herein, with or without at least one additional therapeutic agent, is formulated as a sterile, isotonic solution, properly preserved. In some embodiments, the preparation involves the formulation of the desired molecule with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the engineered T cells are administered at a therapeutically effective amount. For example, a therapeutically effective amount of the engineered T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the therapeutically effective amount of the engineered viable T cells is between about 1×10⁶ and about 2×10⁶ engineered viable T cells per kg body weight up to a maximum dose of about 1×10⁸ engineered viable T cells.

In some embodiments, the engineered T cells are anti-CD19 CART T cells. In some embodiments, the anti-CD19 CAR T cells are the axicabtagene ciloleucel product, YESCARTA™ axicabtagene ciloleucel (axi-cel), TECARTUS™-brexucabtagene autoleucel/KTE-X19, KYIVIRIAH™ (ti sagenlecleucel), lisocabtagene maraleucel, In some embodiments, the engineered T cells are anti-BCMA CAR T cells, such as Idecabtagene vicleucel/bb2121 etc, In some embodiments, the product meets commercial specifications. In some embodiments, the product does not meet commercial specifications (out-of-specification product, OOS). In some embodiments, the OOS product comprises fewer, less differentiated CCR7+ T_{N} and T_{CM} and a greater proportion of more differentiated CCR7−T_{EM}+ T_{EFF} cells than the axicabtagene ciloleucel product that meets commercial specifications. In some embodiments, the OOS product results in a median peak CAR T cell level after administration that is lower than that of the commercial product. In some embodiments, the OOS product still showed a manageable safety profile and meaningful clinical benefit.

The application also provides dosages and administrations of cells prepared by the methods of the application, for example, an infusion bag of CD19-directed genetically modified autologous T cell immunotherapy, comprises a suspension of chimeric antigen receptor (CAR)-positive T cells in approximately 68 mL for infusion. In some embodiments, the CAR T cells are formulated in approximately 40 mL for infusion In some embodiments, the CAR T cell product is formulated in a total volume of 35, 40, 45, 50, 55, 60, 65, 70, 75, 80, 85, 90, 95, 100, 200, 300, 400, 500, 500, 700, 800, 900, 1000 mL. In one aspect, the dosage and administration of cells prepared by the methods of the application, for example, an infusion bag of CD19-directed genetically modified autologous T cell immunotherapy, comprises a suspension of 1×10⁶ CAR-T positive cells in approximately 40 mL. The target dose may be between about 1×10⁶ and about 2×10⁶ CAR-positive viable T cells per kg body weight, with a maximum of 2×10⁸ CAR-positive viable T cells.

In some embodiments, the dosage form comprises a cell suspension for infusion in a single-use, patient-specific infusion bag; the route of administration is intravenous; the entire contents of each single-use, patient-specific bag is infused by gravity or a peristaltic pump over 30 minutes. In one embodiment, the dosing regimen is a single infusion consisting of 2.0×10⁶ anti-CD19 CART cells/kg of body weight (±20%), with a maximum dose of 2×10⁸ anti-CD19 CAR T cells (for subjects ≥100 kg). In some embodiments, the T cells that make up the dose are CD19 CAR-T cells.

In some embodiments, the CD19-directed T cell immunotherapy is KTE-X19, which is prepared as described elsewhere in this application. In one embodiment, KTE-X19 may be used for treatment of MCL, ALL, CLL, SLL, and any other B cell malignancy. In some embodiment, the CD19-directed genetically modified autologous T cell immunotherapy is Axi-cel™ (YESCARTA®, axicabtagene ciloleucel) prepared by one of the methods of the application. Amounts of CART cells, dosage regimens, methods of administration, subjects, cancers, that fall within the scope of these methods are described elsewhere in this application, alone or in combination with another chemotherapeutic agent, with or without preconditioning, and to any of the patients described elsewhere in the application

Conditioning Agents

In some embodiments, the subject is administered a conditioning agent prior to immunotherapy. In some embodiments, conditioning is done with radiation treatment. In some embodiments, the conditioning therapy is a lymphodepleting chemotherapy.

In one embodiment, the conditioning therapy comprises an alkylating agent selected from the group consisting of melphalan, chlorambucil, cyclophosphamide, mechlorethamine, mustine (HN2), uramustine, uracil mustard, melphalan, chlorambucil, ifosfamide, bendamustine, carmustine, lomustine, streptozocin, alkyl sulfonates, busulfan, thiotepa or its analogues, and any combination thereof; a purine analogs selected from the group consisting of azathioprine, 6-mercaptopurine, mercaptopurine, thiopurines, thioguanine, fludarabine, pentostatin, cladribine, and any combination thereof; and/or a platinum-based preconditioning agents selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, and any combination thereof.

Cyclophosphamide (ENDOXAN®, CYTOXAN®, PROCYTOX®, NEOSAR®, REVIMMUNE®, CYCLOBLASTIN®) is a nitrogen mustard-derivative alkylating agent with potent immunosuppressive activity. Cyclophosphamide acts as an antineoplastic, and it is used to treat various types of cancers including lymphoma, multiple myeloma, leukemia, mycosis fungoides, neuroblastoma, ovarian cancer, eye cancer, and breast cancer, as well as autoimmune disorders.

Once administered to a patient, cyclophosphamide is converted into acrolein and phosphoramide in the liver. Together, these metabolites crosslink DNA in both resting and dividing cells by adding an alkyl group to guanine bases of DNA at the number seven nitrogen atom of the imidazole ring. As a result, DNA replication is inhibited leading to cell death.

In another embodiment, the one or more preconditioning agents can include platinum-based chemotherapeutic agents. In certain embodiments, the platinum-based chemotherapeutic agents are selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, any analogues or functional derivatives thereof, and any combination thereof.

In another embodiment, the one or more preconditioning agents can include purine analogues. In certain embodiments, the purine analogues are selected from the group consisting of azathioprine, 6-mercaptopurine, mercaptopurine, thiopurines, thioguanine, fludarabine, pentostatin, cladribine, any analogue or functional derivative thereof, and any combination thereof. In one embodiment, the one or more preconditioning agents includes fludarabine.

Fludarabine phosphate (FLUDARA®) is a synthetic purine nucleoside that differs from physiologic nucleosides in that the sugar moiety is arabinose instead of ribose or deoxyribose. Fludarabine acts as a purine antagonist antimetabolite, and it is used to treat various types of hematological malignancies, including various lymphomas and leukemias.

Once administered to a patient, fludarabine is rapidly dephosphorylated to 2-fluoro-ara-A and then phosphorylated intracellularly by deoxycytidine kinase to the active triphosphate, 2-fluoro-ara-ATP. This metabolite then interferes with DNA replication, likely by inhibiting DNA polymerase alpha, ribonucleotide reductase, and DNA primase, thus inhibiting DNA synthesis. As a result, fludarabine administration leads to increased cell death in dividing cells.

In some embodiments, the one or more preconditioning agents can include cyclophosphamide and a purine analog. The purine analogues can be selected from the group consisting of azathioprine, 6-mercaptopurine, mercaptopurine, thiopurines, thioguanine, fludarabine, pentostatin, cladribine, any analogue or functional derivative thereof, and any combination thereof. In one particular embodiment, the one or more preconditioning agents include cyclophosphamide and pentostatin. In one particular embodiment, the one or more preconditioning agents include cyclophosphamide and fludarabine.

In certain embodiments, a first dose (also applies to repeated doses) of the one or more preconditioning agents is administered to the patient. For example, in some embodiments, a first dose of cyclophosphamide is about 300 mg/m²/day to about 2000 mg/m²/day. In another embodiment, the first dose of cyclophosphamide is higher than 300 mg/m²/day and lower than 2000 mg/m²/day. In other embodiments, the dose of cyclophosphamide is about 350 mg/m²/day-about 2000 mg/m²/day, at least about 400 mg/m²/day-about 2000 mg/m²/day, about 450 mg/m²/day-about 2000 mg/m²/day, about 500 mg/m²/day-about 2000 mg/m²/day, about 550 mg/m²/day-about 2000 mg/m²/day, or about 600 mg/m²/day-about 2000 mg/m²/day. In other embodiments, the dose of cyclophosphamide is about 350 mg/m²/day-about 1500 mg/m²/day, about 350 mg/m²/day-about 1000 mg/m²/day, about 400 mg/m²/day-about 900 mg/m²/day, about 450 mg/m²/day-about 800 mg/m²/day, about 450 mg/m²/day-about 700 mg/m²/day, about 500 mg/m²/day-about 600 mg/m²/day, or about 300 mg/m²/day-about 500 mg/m²/day. In another embodiment, the dose of cyclophosphamide is about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day.

In other embodiments, the first dose (also applies to repeated doses) of cyclophosphamide is about 200 mg/m²/day to about 3000 mg/m²/day. In another embodiment, the first dose of cyclophosphamide is higher than 200 mg/m²/day and lower than 3000 mg/m²/day. In other embodiments, the dose of cyclophosphamide is about 200 mg/m²/day-about 3000 mg/m²/day, about 300 mg/m²/day-about 3000 mg/m²/day, about 400 mg/m²/day-about 3000 mg/m²/day, about 500 mg/m²/day-about 3000 mg/m²/day, about 600 mg/m²/day-about 3000 mg/m²/day, about 700 mg/m²/day-about 3000 mg/m²/day, about 800 mg/m²/day-about 3000 mg/m²/day, about 900 mg/m²/day-about 3000 mg/m²/day, about 1000 mg/m²/day-about 3000 mg/m²/day, about 1100 mg/m²/day-about 3000 mg/m²/day, about 1200 mg/m²/day-about 3000 mg/m²/day, about 1300 mg/m²/day-about 3000 mg/m²/day, about 1400 mg/m²/day-about 3000 mg/m²/day, about 1500 mg/m²/day-about 3000 mg/m²/day, about 1600 mg/m²/day-about 3000 mg/m²/day, about 1700 mg/m²/day-about 3000 mg/m²/day, about 1800 mg/m²/day-about 3000 mg/m²/day, about 1900 mg/m²/day-about 3000 mg/m²/day, about 2000 mg/m²/day-about 3000 mg/m²/day, about 200 mg/m²/day-about 2900 mg/m²/day, about 400 mg/m²/day-about 2800 mg/m²/day, about 500 mg/m²/day-about 2700 mg/m²/day, about 600 mg/m²/day-about 2600 mg/m²/day, about 700 mg/m²/day-about 2500 mg/m²/day, about 800 mg/m²/day-about 2400 mg/m²/day, about 900 mg/m²/day-about 2350 mg/m²/day, about 1000 mg/m²/day-about 2300 mg/m²/day, about 1100 mg/m²/day-about 2250 mg/m²/day, or about 1110 mg/m²/day-about 2220 mg/m²/day. In one embodiment, the first dose of cyclophosphamide is 200 mg/m²/day. In another embodiment, the first dose of cyclophosphamide is 300 mg/m²/day. In another embodiment, the first dose of cyclophosphamide is 500 mg/m²/day.

In some embodiments, a first dose (also applies to repeated doses) of fludarabine is about 20 mg/m²/day to about 900 mg/m²/day. In some embodiments, a dose of fludarabine is higher than 30 mg/m²/day and lower than 900 mg/m²/day. In some embodiments, a dose fludarabine is about 35 mg/m²/day-about 900 mg/m²/day, about 40 mg/m²/day-about 900 mg/m²/day, about 45 mg/m²/day-about 900 mg/m²/day, about 50 mg/m²/day-about 900 mg/m²/day, about 55 mg/m²/day-about 900 mg/m²/day, or about 60 mg/m²/day-about 900 mg/m²/day. In some embodiments, a dose of fludarabine is about 35 mg/m²/day-about 900 mg/m²/day, about 35 mg/m²/day-about 800 mg/m²/day, about 35 mg/m²/day-about 700 mg/m²/day, about 35 mg/m²/day-about 600 mg/m²/day, about 35 mg/m²/day-about 500 mg/m²/day, about 35 mg/m²/day-about 400 mg/m²/day, about 35 mg/m²/day-about 300 mg/m²/day, about 35 mg/m²/day-about 200 mg/m²/day, about 35 mg/m²/day-about 100 mg/m²/day, about 40 mg/m²/day-about 90 mg/m²/day, about 45 mg/m²/day-about 80 mg/m²/day, about 45 mg/m²/day-about 70 mg/m²/day, or about 50 mg/m²/day-about 60 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, about 100 mg/m²/day, about 200 mg/m²/day, or about 300 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In other embodiments, the dose of fludarabine is about 110 mg/m²/day, 120 mg/m²/day, 130 mg/m²/day, 140 mg/m²/day, 150 mg/m²/day, 160 mg/m²/day, 170 mg/m²/day, 180 mg/m²/day, or 190 mg/m²/day. In some embodiments, the dose of fludarabine is about 210 mg/m²/day, 220 mg/m²/day, 230 mg/m²/day, 240 mg/m²/day, 250 mg/m²/day, 260 mg/m²/day, 270 mg/m²/day, 280 mg/m²/day, or 290 mg/m²/day. In one particular embodiment, the dose of fludarabine is about 20 mg/m²/day. In one particular embodiment, the dose of fludarabine is about 25 mg/m²/day. In another embodiment, dose of fludarabine is about 30 mg/m²/day. In another embodiment, dose of fludarabine is about 60 mg/m²/day.

In some embodiments, the dose of cyclophosphamide is 100 mg/m²/day (or 110 mg/m²/day, 120 mg/m²/day, 130 mg/m²/day, or 140 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 150 mg/m²/day (or 160 mg/m²/day, 170 mg/m²/day, 180 mg/m²/day, or 190 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is about 200 mg/m²/day (or 210 mg/m²/day, 220 mg/m²/day, 230 mg/m²/day, or 240 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 250 mg/m²/day (or 260 mg/m²/day, 270 mg/m²/day, 280 mg/m²/day, or 290 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 300 mg/m²/day (or 310 mg/m²/day, 320 mg/m²/day, 330 mg/m²/day, or 340 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 350 mg/m²/day (or 360 mg/m²/day, 370 mg/m²/day, 380 mg/m²/day, or 390 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 400 mg/m²/day (or 410 mg/m²/day, 420 mg/m²/day, 430 mg/m²/day, or 440 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 450 mg/m²/day (or 460 mg/m²/day, 470 mg/m²/day, 480 mg/m²/day, or 490 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 500 mg/m²/day (or 510 mg/m²/day, 520 mg/m²/day, 530 mg/m²/day, or 540 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 550 mg/m²/day (or 560 mg/m²/day, 570 mg/m²/day, 580 mg/m²/day, or 590 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 600 mg/m²/day (or 610 mg/m²/day, 620 mg/m²/day, 630 mg/m²/day, or 640 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 650 mg/m²/day (or 660 mg/m²/day, 670 mg/m²/day, 680 mg/m²/day, or 690 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 700 mg/m²/day (or 710 mg/m²/day, 720 mg/m²/day, 730 mg/m²/day, or 740 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 750 mg/m²/day (or 760 mg/m²/day, 770 mg/m²/day, 780 mg/m²/day, or 790 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 800 mg/m²/day (or 810 mg/m²/day, 820 mg/m²/day, 830 mg/m²/day, or 840 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 850 mg/m²/day (or 860 mg/m²/day, 870 mg/m²/day, 880 mg/m²/day, or 890 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 900 mg/m²/day (or 910 mg/m²/day, 920 mg/m²/day, 930 mg/m²/day, or 940 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 950 mg/m²/day (or 960 mg/m²/day, 970 mg/m²/day, 980 mg/m²/day, or 990 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 1000 mg/m²/day (or 1010 mg/m²/day, 1020 mg/m²/day, 1030 mg/m²/day, or 1040 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In other embodiments, the dose of cyclophosphamide is between 100 mg/m²/day and 650 mg/m²/day, and the dose of fludarabine is between 10 mg/m²/day and 50 mg/m²/day. In other embodiments, the dose of cyclophosphamide is between 150 mg/m²/day and 600 mg/m²/day, and the dose of fludarabine is between 20 mg/m²/day and 50 mg/m²/day. In other embodiments, the dose of cyclophosphamide is between 200 mg/m²/day and 550 mg/m²/day, and the dose of fludarabine is between 20 mg/m²/day and 40 mg/m²/day. In other embodiments, the dose of cyclophosphamide is between 250 mg/m²/day and 550 mg/m²/day, and the dose of fludarabine is between 15 mg/m²/day and 45 mg/m²/day.

In certain embodiments, the dose of cyclophosphamide is 1000 mg/m²/day, and the dose of fludarabine is 60 mg/m²/day, 65 mg/m²/day, 70 mg/m²/day, 75 mg/m²/day, 80 mg/m²/day, 85 mg/m²/day, 90 mg/m²/day, 95 mg/m²/day, 100 mg/m²/day, 105 mg/m²/day, 110 mg/m²/day, 115 mg/m²/day, 120 mg/m²/day, 125 mg/m²/day, 130 mg/m²/day, 135 mg/m²/day, 140 mg/m²/day, 145 mg/m²/day, 150 mg/m²/day, 155 mg/m²/day, 160 mg/m²/day, 165 mg/m²/day, 170 mg/m²/day, 175 mg/m²/day, 180 mg/m²/day, 185 mg/m²/day, 190 mg/m²/day, 195 mg/m²/day, 200 mg/m²/day, 205 mg/m²/day, 210 mg/m²/day, 215 mg/m²/day, 220 mg/m²/day, 225 mg/m²/day, 230 mg/m²/day, 235 mg/m²/day, 240 mg/m²/day, 245 mg/m²/day, or 250 mg/m²/day.

In one embodiment, a dose of cyclophosphamide is about 500 mg/m²/day and a dose of fludarabine is about 60 mg/m²/day. In another embodiment, a dose of cyclophosphamide is about 300 mg/m²/day and a dose of fludarabine is about 30 mg/m²/day. In another embodiment, a dose of cyclophosphamide is about 200 mg/m²/day and a dose of fludarabine is about 20 mg/m²/day. In another embodiment, a dose of cyclophosphamide is about 200 mg/m²/day and a dose of fludarabine is about 30 mg/m²/day. In another embodiment, a dose of cyclophosphamide is about 500 mg/m²/day and a dose of fludarabine is about 30 mg/m²/day. In another embodiment, a dose of cyclophosphamide is about 300 mg/m²/day and a dose of fludarabine is about 60 mg/m²/day. In another embodiment, a dose of cyclophosphamide is about 500 mg/m²/day and a dose of fludarabine is about 60 mg/m²/day. In another embodiment, a dose of cyclophosphamide is about 1110 mg/m²/day and a dose of fludarabine is about 25 mg/m²/day. In another embodiment, a dose of cyclophosphamide is about 2220 mg/m²/day and a dose of fludarabine is about 25 mg/m²/day. In another embodiment, a dose of cyclophosphamide is about 500 mg/m²/day and a dose of fludarabine is about 30 mg/m²/day.

The timing of the administration of the one or more preconditioning agents can be adjusted to maximize effect. In certain embodiments, the one or more preconditioning agents comprise at two or more preconditioning agents. The two or more preconditioning agents can be administered concurrently or sequentially. In one particular embodiment, a first preconditioning agent, e.g., cyclophosphamide, is administered to the patient prior to or after a second preconditioning agent, e.g., fludarabine.

The doses of cyclophosphamide and fludarabine can be raised or lowered together or independently. For example, the dose of cyclophosphamide can be increased while the dose of fludarabine is decreased, and the dose of cyclophosphamide can be decreased while the dose of fludarabine is increased. Alternatively, the dose of both cyclophosphamide and fludarabine can be increased or decreased together. In some embodiments, the dose of cyclophosphamide is 300 mg/m²/day and the dose of fludarabine is 20 mg/m²/day. In other embodiments, the dose of cyclophosphamide is 300 mg/m²/day and the dose of fludarabine is 30 mg/m²/day. In other embodiments, the dose of cyclophosphamide is 300 mg/m²/day and the dose of fludarabine is 60 mg/m²/day. In other embodiments, the dose of cyclophosphamide is 500 mg/m²/day and the dose of fludarabine is 20 mg/m²/day. In other embodiments, the dose of cyclophosphamide is 500 mg/m²/day and the dose of fludarabine is 30 mg/m²/day. In other embodiments, the dose of cyclophosphamide is 500 mg/m²/day and the dose of fludarabine is 60 mg/m²/day. In other embodiments, the dose of cyclophosphamide is 200 mg/m²/day and the dose of fludarabine is 20 mg/m²/day. In other embodiments, the dose of cyclophosphamide is 200 mg/m²/day and the dose of fludarabine is 30 mg/m²/day. In other embodiments, the dose of cyclophosphamide is 200 mg/m²/day and the dose of fludarabine is 60 mg/m²/day.

As described herein, the day that a T cell therapy is administered is designated as day 0. The one or more preconditioning agents can be administered at any time prior to administration of the T cell therapy. In some embodiments, the administration of the one or more preconditioning agents begins at least seven days, at least six days, at least five days, at least four days, at least three days, at least two days, or at least one day prior to the administration of the T cell therapy. In other embodiments, the administration of the one or more preconditioning agents begins at least eight days, at least nine days, at least ten days, at least eleven days, at least twelve days, at least thirteen days, or at least fourteen days prior to the administration of the T cell therapy. In one embodiment, the administration of the one or more preconditioning agents begins about seven days prior to the administration of the T cell therapy. In another embodiment, the administration of the one or more preconditioning agents begins about five days prior to the administration of the T cell therapy.

In one embodiment, the administration of a first preconditioning agent begins about seven days prior to the administration of the T cell therapy, and the administration of a second preconditioning agent begins about five days prior to administration of the T cell therapy. In one particular embodiment, a first preconditioning agent is administered to the patient for two days at about seven days and about six days prior to the administration of the T cell therapy. In another embodiment, a second preconditioning agent is administered to the patient for five days at about five, four, three, two, and one day prior to the administration of the T cell therapy. In another embodiment, a first preconditioning agent is administered to the patient for three days at about five, four, and three days prior to the administration of the T cell therapy.

In one particular embodiment, administration of the cyclophosphamide begins about seven days prior to the administration of the T cell therapy, and the administration of a purine analog (e.g., fludarabine or pentostatin) begins about five days prior to the administration of the T cell therapy. In another embodiment, administration of the cyclophosphamide begins about five days prior to the administration of the T cell therapy, and the administration of a purine analog (e.g., fludarabine or pentostatin) begins about five days prior to the administration of the T cell therapy.

The timing of the administration of each component can be adjusted to maximize effect. In general, the one or more preconditioning agents can be administered daily. In some embodiments, the one or more preconditioning agents are administered daily for about two days, for about three days, for about four days, for about five days, for about six days, or for about seven days. In some embodiments, the one or more preconditioning agents can be administered daily for at least one day, at least two days, at least three days, at least four days, at least five days, at least six days, or at least seven days. In one particular embodiment, the one or more preconditioning agents are administered daily for about three days.

As described herein, the day the T cell therapy is administered to the patient is designated as day 0. In some embodiments, the one or more preconditioning agents, e.g., the cyclophosphamide, is administered to the patient on day 7 and day 6 prior to day 0 (i.e., day −7 and day −6). In other embodiments, the one or more preconditioning agents, e.g., the cyclophosphamide, is administered to the patient on day −5, day −4, and day −3. In some embodiments, the one or more preconditioning agents, e.g., the fludarabine, is administered to the patient on day −5, day −4, day −3, day −2, and day −1. In other embodiments, the one or more preconditioning agents, e.g., fludarabine, is administered to the patient on day −5, day −4, and day −3.

The one or more preconditioning agents, e.g., the cyclophosphamide and fludarabine, can be administered on the same or different days. If cyclophosphamide and fludarabine are administered on the same day, the cyclophosphamide dose can be administered either before or after the fludarabine dose. In one embodiment, the cyclophosphamide dose is administered to the patient on day −7 and day −6, and the fludarabine dose is administered to the patient on day −5, day −4, day −3, day −2, and day −1. In another embodiment, the cyclophosphamide dose is administered to the patient on day −5, day −4, and day −3, and the fludarabine dose is administered to the patient on day −5, day −4, and day −3.

In certain embodiments, the one or more preconditioning agents, e.g., cyclophosphamide and fludarabine, can be administered concurrently or sequentially. In one embodiment, cyclophosphamide is administered to the patient prior to fludarabine. In another embodiment, cyclophosphamide is administered to the patient after fludarabine.

The one or more preconditioning agents can be administered by any route, including intravenously (IV) or orally. In some embodiments, the one or more preconditioning agents, e.g., the cyclophosphamide, is administered by IV over about 30 minutes, over about 35 minutes, over about 40 minutes, over about 45 minutes, over about 50 minutes, over about 55 minutes, over about 60 minutes, over about 90 minutes, over about 120 minutes. In some embodiments, the one or more preconditioning agents, e.g., the fludarabine, is administered by IV over about 10 minutes, over about 15 minutes, over about 20 minutes, over about 25 minutes, over about 30 minutes, over about 35 minutes, over about 40 minutes, over about 45 minutes, over about 50 minutes, over about 55 minutes, over about 60 minutes, over about 90 minutes, over about 120 minutes

In another embodiment, a lymphodepleting chemotherapy regimen of cyclophosphamide 500 mg/m² intravenously and fludarabine 30 mg/m² is administered intravenously on the fifth, fourth, and third day before infusion of the CAR T cells to the subject. In some embodiments, high- or low-intensity lymphodepletion chemotherapy with a Cy/Flu-based regimen (the high-intensity regimen includes cyclophosphamide at 60 mg/kg; the low-intensity regimen includes cyclophosphamide ≤1500 mg/m² or 30 mg/kg total dose). In some embodiments, the conditioning regimen consists of fludarabine 30 mg/m² and cyclophosphamide 300 mg/m² daily for 3 days (e.g., with the last day occurring on day −7 to −2 prior to CAR T cell infusion. In some embodiments, the conditioning regimen consists of fludarabine (30 mg/m² per day) and cyclophosphamide (300 mg/m²) on days −5, −4, and −3 prior to CAR T cell infusion.

### Cancers

The methods disclosed herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In some embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CIVIL), chronic myeloid leukemia (CIVIL), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, Waldenstrom macroglobulinemia, DLBCL arising from FL, high grade B cell lymphoma, or a combination thereof.

In some embodiments, the cancer is a myeloma. In some embodiments, the cancer is multiple myeloma. In some embodiments, the cancer is leukemia. In some embodiments, the cancer is acute myeloid leukemia. In some embodiments, the cancer is relapsed or refractory large B-cell lymphoma (possibly, after two or more lines of systemic therapy), including diffuse large B-cell lymphoma (DLBCL) not otherwise specified, primary mediastinal large B-cell lymphoma, high grade B-cell lymphoma, and DLBCL arising from follicular lymphoma, or relapsed or refractory follicular lymphoma (FL) (possibly, after two or more lines of systemic therapy), or relapsed or refractory mantle cell lymphoma (MCL).

In some embodiments, the cancer is Non-Hodgking lymphoma. In some embodiments, the cancer is relapsed/refractory NHL. In some embodiments, the cancer is mantle cell lymphoma.

In some embodiments, the cancer is advanced-stage indolent non-Hodgkin lymphoma (iNHL), including follicular lymphoma (FL) and marginal zone lymphoma (MZL). In some embodiments, the patient has had relapsed/refractory disease after ≥2 prior lines of therapy, including an anti-CD20 monoclonal antibody with an alkylating agent. In some embodiments, the patient may have received a PI3K inhibitor. In some embodiments, the patient may (also) have received autologous stem cell transplantation. In some embodiments, the patient undergoes leukapheresis to obtain T cells for CAR T cell manufacturing, followed by conditioning chemotherapy with cyclophosphamide at 500 mg/m²/day and fludarabine at 30 mg/m²/day administered on days −5, −4, and −3; on day 0, the patient may receive a single intravenous infusion of CAR T cell therapy (e.g., axicabtagene ciloleucel) at a target dose of 2×10⁶ CAR T cells/kg. In some embodiments, additional infusions may be given at a later period. In some embodiments, if the patient progresses after responding at the month 3 assessment after initial administration, the patient may receive retreatment with CAR T cell treatment (e.g., axicabtagene ciloleucel). In some embodiments, the patient may receive bridging therapy. Examples of bridging therapies are provided elsewhere in the specification, including the Examples. In some embodiments, the patient experiences CRS. In some embodiments, CRS is managed using any one of the protocols described in this application, including the Examples. In some embodiments, CRS is managed with tocilizumab, corticosteroids and/or vasopressor.

In some embodiments, the cancer is relapsed/refractory indolent Non-Hodgkin Lymphoma and the method of treating a subject in need thereof comprises administering to the subject a therapeutically effective amount of CAR T cells as a retreatment, wherein the subject has previously received a first treatment with CAR T cells. In some embodiments, the first treatment with CAR T cells may have been administered as a first line therapy or a second line therapy, optionally wherein the lymphoma is R/R follicular lymphoma (FL) or marginal zone lymphoma (MZL) and optionally wherein the previous prior lines of therapy included anti-CD20 monoclonal antibody combined with an alkylating agent. In some embodiments, the conditioning therapy comprises fludarabine 30 mg/m² IV and cyclophosphamide 500 mg/m² IV on Days −5, −4, and −3. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 109, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cells are axicabtagene ciloleucel CAR T cells. In some embodiments, the retreatment eligibility criteria include response of a CR or PR at the month 3 disease assessment with subsequent progression; no evidence of CD19 loss in progression biopsy by local review; and/or no Grade 4 CRS or neurologic events, or life-threatening toxicities with the first treatment with CAR T cells. In some embodiments, the method of treatment is that followed by the ZUMA-5 clinical trial (NCT03105336).

In some embodiments, the cancer is NHL and the immunotherapy (e.g, CAR T or TCR T cell treatment) is administered as a first line therapy. In some embodiments, the cancer is LBCL. In some embodiments, the LBCL is high risk/high grade LBCL with MYC and BCL2 and/or BCL6 translocations or DLBCL with IPI score ≥3 any time before enrollment. In some embodiments, the first line therapy comprises CAR T cell treatment in combination with an anti-CD20 monoclonal antibody and anthracycline-containing regimen. In some embodiments, the CAR T cell treatment is administered first. In some embodiments, the anti-CD20 monoclonal antibody/anthracycline-containing regimen is administered first. In some embodiments, the treatments are administered at least 2 weeks, at least 4 weeks, at least 6 weeks, at least 1 month, at least 2 months, at least 3 months, at least 4 months, at least 5 months, less than a year apart, etc. In some embodiments, the method further comprises bridging therapy administered after leukapheresis and completed prior to initiating conditioning chemotherapy. In some embodiments, additional inclusion criteria include age ≥18 years and ECOG PS 0-1. In some embodiments, the conditioning therapy comprises fludarabine 30 mg/m² IV and cyclophosphamide 500 mg/m² IV on Days −5, −4, and −3. Other exemplary beneficial preconditioning treatment regimens are described in U.S. Provisional Patent Applications 62/262,143 and 62/167,750 and U.S. Pat. Nos. 9,855,298 and 10,322,146, which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. Another embodiment comprises serum cyclophosphamide and fludarabine at days −4, −3, and −2 prior to T cell administration at a dose of 500 mg/m² of body surface area of cyclophosphamide per day and a dose of 30 mg/m² of body surface area per day of fludarabine during that period of time. Another embodiment comprises cyclophosphamide at day −2 and fludarabine at days −4, −3, and −2 prior to T cell administration, at a dose of 900 trig/m² of body surface area of cyclophosphamide and a dose of 2.5 mg/m² of body surface area per day of fludarabine during that period of time. In another embodiment, the conditioning comprises cyclophosphamide and fludarabine at days −5, −4 and −3 prior to T cell administration at a dose of 500 mg/m² of body surface area of cyclophosphamide per day and a dose of 30 mg/m² of body surface area of fludarabine per day during that period of time. Other preconditioning embodiments comprise 200-300 mg/m² of body surface area of cyclophosphamide per day and a dose of 20-50 mg/m² of body surface area per day of fludarabine for three days. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cell treatment comprises anti-CD19 CAR T cells. In some embodiments, the CAR T cell treatment comprises axicabtagene ciloleucel or YESCARTA™. In some embodiments, the CAR T cell treatment comprises TECARTUS™-brexucabtagene autoleucel/KTE-X19 or KYMRIAH™ (tisagenlecleucel), etc), Idecabtagene vicleucel/bb2121. In some embodiments, the method of treatment is the method used in any one of the ZUMA-1 through ZUMA-19, KITE-585, KITE-222, KITE-037, KITE-363, KITE-439, or KITE-718 clinical trials, which are well-described in the art.

In another embodiment, the disclosure provides a method of treating cancer in a subject in need thereof, comprising administering a therapeutically effective amount of CD19 CAR-T treatment to a subject in which the number of lines of prior therapy are 1-2; 3; 4; or 5. In one embodiment, the disclosure provides a method of treating cancer in a subject in need thereof, comprising administering a therapeutically effective amount of CD19 CAR-T treatment to a subject in which the number of lines of prior therapy are 1-2. The cancer may be any one of the above listed cancers. The CD19 CAR-T treatment may be any one of the above listed CD19 CAR-T treatments. In some embodiments, the CD19 CAR-T treatment is used as first line of treatment. In some embodiments, the CD19 CAR-T treatment is used as a second line of treatment.

In one embodiment, the CD19 CAR-T treatment is any of the of CD19 CAR-T treatments described above. In one embodiment, the CD19 CAR-T treatment comprises axicabtagene ciloleucel treatment. In embodiments, the cancer is refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In one embodiment, a subject selected for axicabtagene ciloleucel treatment has refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In some embodiments, axicabtagene ciloleucel treatment is used as a second line of treatment, where the first line therapy is CHOP, i.e., Cyclophosphamide (Cytoxan®), Doxorubicin (hydroxydoxorubicin), Vincristine (Oncovin®), and Prednisone. In some embodiments, axicabtagene ciloleucel treatment is used as a second line of treatment, where the first line therapy is R-CHOP (CHOP plus Rituximab).

In embodiments, a patient is selected for second-line axicabtagene ciloleucel treatment that has relapsed or refractory disease after first-line chemoimmunotherapy, refractory disease defined as no complete remission to first-line therapy; individuals who are intolerant to first-line therapy are excluded. progressive disease (PD) as best response to first-line therapy, stable disease (SD) as best response after at least 4 cycles of first-line therapy (eg, 4 cycles of R-CHOP), partial response (PR) as best response after at least 6 cycles and biopsy-proven residual disease or disease progression ≤12 months of therapy, and/or relapsed disease defined as complete remission to first-line therapy followed by biopsy-proven relapse ≤12 months of first-line therapy. In some embodiments, a patient selected for second-line axicabtagene ciloleucel treatment is provided conditioning therapy comprising fludarabine 30 mg/m² IV and cyclophosphamide 500 mg/m² IV on Days −5, −4, and −3. In some embodiments, axicabtagene ciloleucel treatment is used as a second line of treatment.

Combination Treatments

Compositions comprising CAR-expressing immune effector cells disclosed herein may be administered in conjunction (before, after, and/or concurrently with T cell administration) with any number of chemotherapeutic agents. In some embodiments, the antigen binding molecule, transduced (or otherwise engineered) cells (such as CARs), and the chemotherapeutic agent are administered each in an amount effective to treat the disease or condition in the subject. Examples of chemotherapeutic agents include alkylating agents such as thiotepa and cyclophosphamide (CYTOXAN′); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylol melamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; Polysaccharide K (PSK); razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone, R-CHOP (CHOP plus Rituximab), and G-CHOP (CHOP plus obinutuzumab).

In some embodiments, the chemotherapeutic agent is administered at the same time or within one week after the administration of the engineered cell. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein (before, after, and/or concurrently with T cell administration). For example, potentially useful additional therapeutic agents include PD-1 inhibitors such as nivolumab (OPDIVO®), pembrolizumab (KEYTRUDA®), Cemiplimab (Libtayo), pidilizumab (CureTech), and atezolizumab (Roche), and PD-L1 inhibitors such as atezolizumab, durvalumab, and avelumab. In some embodiments, the therapeutic agent(s) to use in combination is anti-IL-1 (e.g. anakinra), T cell activation inhibitors (e.g. dasatinib), JAK inhibitors (e.g. filgotinib), anti-GM-CSF (e.g. lenzilumab), anti-TNF (e.g. infliximab), Ang2 inhibitors (e.g. azilsartan), anti-angiogenic therapies (e.g. bevacizumab), and/or anti-IFNg (e.g. emapalumab-lzsg)

Additional therapeutic agents suitable for use in combination (before, after, and/or concurrently with T cell administration) with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib), inhibitors of GM-CSF, CSF1, GM-CSFR, or CSF1R, in addition to anti-thymocyte globulin, lenzilumab and mavrilimumab.

In one embodiment, the GM-CSF inhibitor is selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule. In one embodiment, the CSF1 inhibitor is selected from RG7155, PD-0360324, MCS110/lacnotuzumab), or a biosimilar version of any one of the same; and a small molecule. In one embodiment, the GM-CSFR inhibitor and the CSF1R inhibitor is/are selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or R05509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule.

In some embodiments, a composition comprising an immunotherapy (e.g., engineered CAR T cells) is administered with an anti-inflammatory agent (before, after, and/or concurrently with T cell administration). Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the compositions described herein are administered in conjunction with a cytokine (before, after, or concurrently with T cell administration). Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and —II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-C SF (GM-CSF); and granulocyte-C SF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the administration of the cells and the administration of the additional therapeutic agent are carried out on the same day, are carried out no more than 36 hours apart, no more than 24 hours apart, no more than 12 hours apart, no more than 6 hours apart, no more than 4 hours apart, no more than 2 hours apart, or no more than 1 hour apart or no more than 30 minutes apart. In some embodiments, the administration of the cells and the administration of the additional therapeutic agent are carried out between at or about 0 and at or about 48 hours, between at or about 0 and at or about 36 hours, between at or about 0 and at or about 24 hours, between at or about 0 and at or about 12 hours, between at or about 0 and at or about 6 hours, between at or about 0 and at or about 2 hours, between at or about 0 and at or about 1 hours, between at or about 0 and at or about 30 minutes, between at or about 30 minutes and at or about 48 hours, between at or about 30 minutes and at or about 36 hours, between at or about 30 minutes and at or about 24 hours, between at or about 30 minutes and at or about 12 hours, between at or about 30 minutes and at or about 6 hours, between at or about 30 minutes and at or about 4 hours, between at or about 30 minutes and at or about 2 hours, between at or about 30 minutes and at or about 1 hour, between at or about 1 hours and at or about 48 hours, between at or about 1 hour and at or about 36 hours, between at or about 1 hour and at or about 24 hours, between at or about 1 hour and at or about 12 hours, between at or about 1 hour and at or about 6 hours, between at or about 1 hour and at or about 4 hours, between at or about 1 hour and at or about 2 hours, between at or about 2 hours and at or about 48 hours, between at or about 2 hours and at or about 36 hours, between at or about 2 hours and at or about 24 hours, between at or about 2 hours and at or about 12 hours, between at or about 2 hours and at or about 6 hours, between at or about 2 hours and at or about 4 hours, between at or about 4 hours and at or about 48 hours, between at or about 4 hours and at or about 36 hours, between at or about 4 hours and at or about 24 hours, between at or about 4 hours and at or about 12 hours, between at or about 4 hours and at or about 6 hours, between at or about 6 hours and at or about 48 hours, between at or about 6 hours and at or about 36 hours, between at or about 6 hours and at or about 24 hours, between at or about 6 hours and at or about 12 hours, between at or about 12 hours and at or about 48 hours, between at or about 12 hours and at or about 36 hours, between at or about 12 hours and at or about 24 hours, between at or about 24 hours and at or about 48 hours, between at or about 24 hours and at or about 36 hours or between at or about 36 hours and at or about 48 hours. In some embodiments, the cells and the additional therapeutic agent are administered at the same time.

In some embodiments, the agent is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg.

In some embodiments, the agent is administered in a dosage amount from 0.5 mg/kg to 100 mg/kg, 1 mg/kg to 50 mg/kg, 1 mg kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount from 1 mg/kg to 10 mg/kg, 2 mg kg/to 8 mg/kg, 2 mg/kg to 6 mg/kg, 2 mg/kg to 4 mg/kg or 6 mg/kg to 8 mg/kg, each In some aspects, the agent is administered in a dosage amount of at least 1 mg/kg, 2 mg/kg, 4 mg/kg, 6 mg/kg, 8 mg/kg, 10 mg/kg or more.

In some embodiments, the agent(s) is/are administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration

In some embodiments, the treatment further comprises bridging therapy, which is therapy between conditioning and the compositions disclosed herein or therapy administered after leukapheresis and completed prior to initiating conditioning chemotherapy. In some embodiments, the bridging therapy comprises, CHOP, G-CHOP, R-CHOP (rituximab, cyclophosphamide, doxorubicin, vincristine, and prednisolone), corticosteroids, bendamustine, platinum compounds, anthracyclines, and/or phosphoinositide 3-kinase (PI3K) inhibitors. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032). In some embodiments, the AKT inhibitor is perifosine, MK-2206. In one embodiment, the mTOR inhibitor is selected from everolimus, sirolimus, temsirolimus, ridaforolimus. In some embodiments, the dual PI3K/mTOR inhibitor is selected from BEZ235, XL765, and GDC-0980. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032).

In some embodiments, the bridging therapy comprises acalabrutinib, brentuximab vedotin, copanlisib hydrochloride, nelarabine, belinostat, bendamustine hydrochloride, carmustine, bleomycin sulfate, bortezomib, zanubrutinib, carmustine, chlorambucil, copanlisib hydrochloride, denileukin diftitox, dexamethasone, doxorubicin hydrochloride, duvelisib, pralatrexate, obinutuzumab, ibritumomab tiuxetan, ibrutinib, idelalisib, recombinant interferon alfa-2b, romidepsin, lenalidomide, mechloretamine hydrochloride, methotrexate, mogamulizumab-kpc, prerixafor, nelarabine, obinutuzumab, denileukin diftitox, pembrolizumab, plerixafor, polatuzumab vedotin-piiq, mogamulizumab-kpc, prednisone, rituximab, hyaluronidase, romidepsin, bortezomib, venetoclax, vinblastine sulfate, vorinostat, zanubrutinib, CHOP, COPP, CVP, EPOCH, R-EPOCH, HYPER-CVAD, ICE, R-ICE, R-CHOP, R-CVP, and combinations of the same.

In some embodiments, the cell immunotherapy is administered in conjunction with debulking therapy, which is used with the aim of reducing tumor burden. In one embodiment, debulking therapy is to be administered after leukapheresis and prior to administration of conditioning chemotherapy or cell infusion. Examples of debulking therapy include the following (Table 1)

TABLE 1
Exemplary debulking bridging therapies

| Type | Proposed Regimen^{a} | Timing/Washout |
|---|---|---|
| R-CHOP | Rituximab 375 mg/m² Day 1 | Should be administered |
| | Doxorubicin 50 mg/m² Day 1 | after leukapheresis/enrollment |
| | Prednisone 100 mg Day 1 | and should be completed |
| | through Day 5 | at least 14 days prior to the start |
| | Cyclophosphamide 750 mg/m² | of conditioning chemotherapy |
| | Day 1 Vincristine 1.4 mg/m² | |
| | Day 1 | |
| R-ICE | Rituximab 375 mg/m² | |
| | Day 1 | |
| | Ifosfamide 5 g/m² 24 h-CI | |
| | Day 2 | |
| | Carboplatin AUC5 Day 2 | |
| | maximum dose 800 mg | |
| | Etoposide 100 mg/m²/d | |
| | Days 1 through Day 3 | |
| R-GEMOX | Rituximab 375 mg/m² Day 1 | |
| | Gemcitabine 1000 mg/m² | |
| | Day 2 Oxaliplatin 100 mg/m² | |
| | Day 2 | |
| R-GDP | Rituximab 375 mg/m² | |
| | Day 1 (or Day 8) | |
| | Gemcitabine 1 g/m² on Day 1 | |
| | and Day 8 Dexamethasone | |
| | 40 mg on Day 1 through | |
| | Day 4 Cisplatin 75 mg/m² on | |
| | Day 1 (or carboplatin AUC5 | |
| | on Day 1) | |
| RADIOTHERAPY^{b} | Per local standard up to 20 to | Should be administered |
| | 30 Gy | after leukapheresis/enrollment |
| | | and should be completed |
| | | at least 5 days prior to the start |
| | | of conditioning chemotherapy |

Abbreviations: AUC, area under the curve
^{a}Other debulking treatment options may be used, to be discussed with the medical monitor. Supportive care with hydration, anti-emesis, mesna, growth factor support, and tumor lysis prophylaxis according to local standard may be used. More than 1 cycle allowed.
^{b}At least 1 target lesion should remain outside of the radiation field to allow for tumor measurements

Monitoring

In some embodiments, administration of the immunotherapy (e.g., chimeric receptor T cell immunotherapy) occurs at a certified healthcare facility.

In some embodiments, the methods disclosed herein comprise monitoring patients at least daily for 7 days at the certified healthcare facility following infusion for signs and symptoms of CRS and neurologic toxicities and other adverse reactions to CAR T cell treatment. In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

### Prevention or Management of Severe Adverse Reactions

In some embodiments, the method comprises management of adverse reactions in any subject. In some embodiments, the adverse reaction is selected from the group consisting of cytokine release syndrome (CRS), a neurologic toxicity, a hypersensitivity reaction, a serious infection, a cytopenia and hypogammaglobulinemia.

In some embodiments, the present disclosure provides methods of preventing the development or reducing the severity of adverse reactions based on the levels of a number of biomarker levels in the serum of the subject undergoing immunotherapy. In some embodiments, the cell therapy is administered in with one or more agents that prevents, delays the onset of, reduces the symptoms of, treats the adverse events, which include cytokine release syndromes and neurologic toxicity. In one embodiment, the agent has been described above. In other embodiments, the agent is described below. In some embodiments, the agent is administered by one of the methods and doses described elsewhere in the specification, before, after, or concurrently with the administration of the cells. In one embodiment, the agent(s) are administered to a subject that may be predisposed to the disease but has not yet been diagnosed with the disease.

In some embodiments, the immunotherapy (e.g., cell treatment) is administered before, during/concurrently, and/or after the administration of one or more agents (e.g., steroids) or treatments (e.g., debulking) that treat and or prevent (are prophylactic) one or more symptoms of adverse events. The pharmacologic and/or physiologic effect may be prophylactic, i.e., the effect completely or partially prevents a disease or symptom thereof. A “prophylactically effective amount” refers to an amount effective, at dosages and for periods of time necessary, to achieve the desired prophylactic result. In one embodiment, a prophylactically effective amount is used in subjects prior to or at an earlier stage of disease. In one embodiment, the prophylactically effective amount will be less than the therapeutically effective amount. In some embodiments, the patient is selected for management of adverse events based on the expression of one of more of the markers described herein in this specification. In one embodiment, the adverse event treatment or prophylaxis is administered to any patient that will receive, is receiving, or has received cell therapy.

In some embodiments, the signs and symptoms of adverse reactions are selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia.

In some embodiments, the patient has been identified and selected based on one or more of the biomarkers described in this application. In some embodiments, the patient has been identified and selected simply by the clinical presentation (e.g., presence and grade of toxicity symptom).

In some embodiments, the adverse events/reactions may be chosen from one or more of the following (Table 2):

TABLE 2
Exemplary adverse events
Adverse Event/Reaction
Blood and Lymphatic System Disorders
Coagulopathy ^{a}
Cardiac Disorders
Tachycardias ^{b}
Bradycardias ^{c}
Non-ventricular Arrhythmias ^{d}
Gastrointestinal Disorders
Nausea
Constipation
Diarrhea
Abdominal pain ^{e}
Oral pain ^{f}
Vomiting ^{g}
Dysphagia
Pyrexia
Fatigue ^{h}
Chills
Edema ⁱ
Dry mouth
Pain ^{j}
Immune System Disorders
Cytokine release syndrome
Hypogammaglobulinemia ^{k}
Infections and Infestations
Infection-pathogen unspecified
Viral infections
Bacterial infections
Metabolism and nutrition disorders
Decreased appetite
Musculoskeletal pain ^{l}
Motor dysfunction ^{m}
Psychiatric Disorders
Nervous System Disorders
Encephalopathy ⁿ
Tremor
Headache ^{o}
Aphasia ^{p}
Dizziness ^{q}
Neuropathy ^{r}
Insomnia
Delirium ^{s}
Anxiety
Immune System Disorders
Cytokine release syndrome
Hypogammaglobulinemia ^{k}
Infections and Infestations
Infection-pathogen unspecified
Viral infections
Bacterial infections
Metabolism and nutrition disorders
Decreased appetite
Musculoskeletal pain ^{l}
Motor dysfunction ^{m}
Psychiatric Disorders
Nervous System Disorders
Encephalopathy ⁿ
Tremor
Headache ^{o}
Aphasia ^{p}
Dizziness ^{q}
Neuropathy ^{r}
Insomnia
Delirium ^{s}
Anxiety
Renal and Urinary Disorders
Renal insufficiency ^{t}
Urine output decreased ^{u}
Hypoxia
Cough ^{v}
Dyspnea ^{w}
Pleural effusion
Skin and Subcutaneous Tissue Disorders
Rash ^{x}
Vascular Disorders
Hypotension ^{y}
Hypertension
Thrombosis ^{z}
Hemorrage

### Cytokine Release Syndrome (CRS)

In some embodiments, the method comprises preventing or reducing the severity of CRS in a chimeric receptor treatment. In some embodiments, the engineered CAR T cells are deactivated after administration to the patient.

In some embodiments, the method comprises identifying CRS based on clinical presentation. In some embodiments, the method comprises evaluating for and treating other causes of fever, hypoxia, and hypotension. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered.

In some embodiments, the method comprises monitoring patients at least daily for 7 days at the certified healthcare facility following infusion for signs and symptoms of CRS. In some embodiments, the method comprises monitoring patients for signs or symptoms of CRS for 4 weeks after infusion. In some embodiments, the method comprises counseling patients to seek immediate medical attention should signs or symptoms of CRS occur at any time. In some embodiments, the method comprises instituting treatment with supportive care, tocilizumab or tocilizumab and corticosteroids as indicated at the first sign of CRS.

In some embodiments, the subject experiences Grade 3+ CRS. In some embodiments, this includes pyrexia, hypotension, tachycardia, hypoxia, chills, sinus tachycardia, fatigue, headache, vomiting, acute kidney injury, myalgia, atrial fibrillation, diarrhea, dyspnea, ejection fraction decreased, pulmonary oedema, atrial flutter, blood creatine increased, capillary leak syndrome, decreased appetite, febrile neutropenia, malaise, metabolic acidosis, fever, nausea, headache, rash, rapid heartbeat, low blood pressure, trouble breathing, etc.

### Neurologic Toxicity (NT)

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the subject experiences Grade 3+NT. In some embodiments, this includes encephalopathy, tremor, confusional state, aphasia, somnolence, agitation, memory impairment, dysarthria, hallucination, mental status changes, ataxia, restlessness, seizure, delirium, disturbance in attention, lethargy, depressed level of consciousness, disorientation, dyscalculia, hemiparesis, monoclonus, cerebral edema, and others

Management of Adverse Events

In some embodiments, the method of managing adverse events comprises monitoring patients at least daily for 7 days at the certified healthcare facility following infusion for signs and symptoms of neurologic toxicities. In some embodiments, the method comprises monitoring patients for signs or symptoms of neurologic toxicities and/or CRS or 4 weeks after infusion

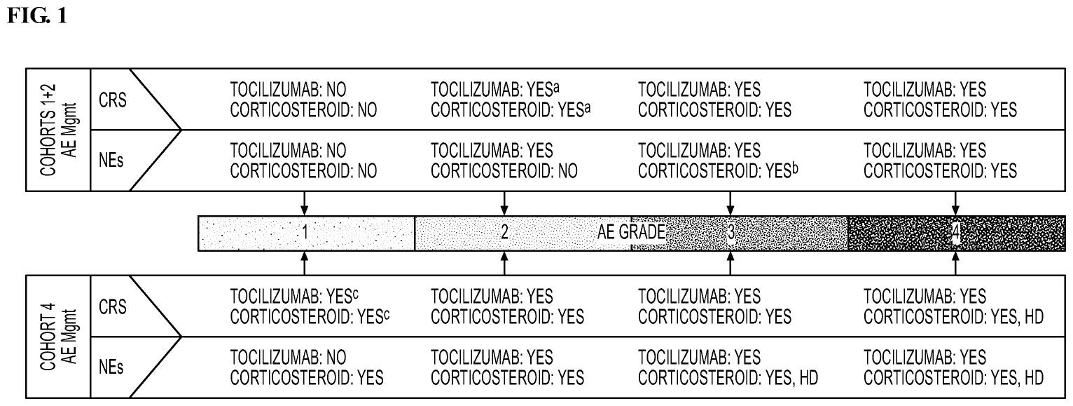

In some embodiments, the disclosure provides two methods of managing adverse events in subjects receiving CAR T cell treatment with steroids and anti-IL6/anti-IL-6R antibody/ies. In one embodiment, the methods are outlined in FIG. 1. In one embodiment, the CAR T cell treatment is an anti-CD19 treatment, as described in the Examples. In one embodiment, the CAR T cell treatment is known as ZUMA-1, which includes different adverse event management protocols for different cohorts. In one embodiment, the disclosure provides that early steroid intervention in Cohort 4 is associated with lower rates of severe CRS and neurologic events than what was observed in Cohorts 1+2. In one embodiment, the disclosure provides that earlier use of steroids in Cohort 4 was associated with a median cumulative cortisone-equivalent dose approximately 15% of that in Cohorts 1+2, suggesting that earlier steroid use may allow reduction of overall steroid exposure. Accordingly, in one embodiment, the disclosure provides a method of adverse event management whereby corticosteroid therapy is initiated for management of all cases of grade 1 CRS if there was no improvement after 3 days and for all grade ≥1 neurologic events. In one embodiment, tocilizumab is initiated for all cases of grade 1 CRS if there is no improvement after 3 days and for all grade ≥2 neurologic events. In one embodiment, the disclosure provides a method of reducing overall steroid exposure in patients receiving adverse event management after CAR T cell administration, the method comprising initiation of corticosteroid therapy for management of all cases of grade 1 CRS if there was no improvement after 3 days and for all grade ≥1 neurologic events and/or initiation of tocilizumab for all cases of grade 1 CRS if there is no improvement after 3 days and for all grade ≥2 neurologic events. In one embodiment, the corticosteroid and tocilizumab are administering in a regimen selected from those exemplified in protocols A through C. In one embodiment, the disclosure provides that earlier steroid use is not associated with increased risk for severe infection, decreased CAR T-cell expansion, or decreased tumor response.

In one embodiment, the disclosure supports the safety of levetiracetam prophylaxis in CAR T cell cancer treatment. In one embodiment, the cancer is NHL. In one embodiment, the cancer is R/R LBCL and the patients receive axicabtagene ciloleucel. Accordingly, in one embodiment, the disclosure provides a method of managing adverse events in patients treated with CAR T cells comprising administering to the patient a prophylactic dosage of an anti-seizure medication. In some embodiments, the patients receive levetiracetam (for example, 750 mg orally or intravenous twice daily) starting on day 0 of the CAR T cell treatment (after conditioning) and also at the onset of grade ≥2 neurologic toxicities, if neurologic events occur after the discontinuation of prophylactic levetiracetam. In one embodiment, if a patient does not experience any grade ≥2 neurologic toxicities, levetiracetam is tapered and discontinued as clinically indicated. In one embodiment, levetiracetam prophylaxis is combined with any other adverse event management protocol.

In one embodiment, the disclosure provides that CAR T-cell levels in the patients subject to the adverse management protocol of Cohort 4 were comparable to those of Cohorts 1+2. In one embodiment, the disclosure provides that the numerical levels of key inflammatory cytokines associated with CAR-related inflammatory events (e.g, IFNγ, IL-2 and GM-CSF) are lower in Cohort 4 than in Cohorts 1+2. Accordingly, the disclosure provides a method of reducing CAR T cell treatment-related inflammatory events without impact on CAR T cell levels comprising administering to the patient the adverse event management protocol of Cohort 4. The disclosure also provides a method of reducing cytokine production by immune cells after CAR T cell therapy comprising administering to the patient the adverse event management protocol of Cohort 4. In one embodiment, this effect is obtained without affecting CAR T-cell expansion and response rates. In one embodiment, the patient has R/R LBCL. In one embodiment, the CAR T cell treatment is anti-CD19 CAR T cell treatment. In one embodiment, the CAR T cell treatment comprises axicabtagene ciloleucel.

In one embodiment, the disclosure provides that early or prophylactic use of tocilizumab following axicabtagene ciloleucel for adverse event management decreased grade ≥3 cytokine release syndrome but increased grade ≥3 neurologic events. Accordingly, the disclosure provides a method for adverse event management in CAR T-cell therapy as described in FIG. 1. In one embodiment, patients receive levetiracetam (750 mg oral or intravenous twice daily) starting on day 0. At the onset of grade ≥2 neurologic events, levetiracetam dose is increased to 1000 mg twice daily. If a patient did not experience any grade ≥2 neurologic event, levetiracetam is tapered and discontinued as clinically indicated. Patients also receive tocilizumab (8 mg/kg IV over 1 hour [not to exceed 800 mg]) on day 2. Further tocilizumab (±corticosteroids) may be recommended at the onset of grade 2 CRS in patients with comorbidities or older age, or otherwise in case of grade ≥3 CRS. For patients experiencing grade ≥2 neurologic events, tocilizumab is initiated, and corticosteroids are added for patients with comorbidities or older age, or if there is any occurrence of a grade ≥3 neurologic event with worsening symptoms despite tocilizumab use.

In one embodiment, the disclosure provides that prophylactic steroid use appears to reduce the rate of severe CRS and NEs to a similar extent as early steroid use following axicabtagene ciloleucel administration. Accordingly, the disclosure provides a method for adverse event management in CAR T-cell therapy wherein patients receive dexamethasone 10 mg PO on Days 0 (prior to axicabtagene ciloleucel infusion), 1, and 2. Steroids are also administered starting at Grade 1 NE, and for Grade 1 CRS when no improvement is observed after 3 days of supportive care. Tocilizumab is also administered for Grade ≥1 CRS if no improvement is observed after 24 hours of supportive care.

In one embodiment, the disclosure provides that adverse event management of CAR T-cell therapy with an antibody that neutralizes and/or depletes GM-C SF prevents or reduces treatment-related CRS and/or NEs in treated patients. In one embodiment, the antibody is lenzilumab.

In one embodiment, the method of prevention and/or management of adverse events comprises administering a “prophylactically effective amount” of tocilizumab, of a corticosteroid therapy, and/or of an anti-seizure medicine for toxicity prophylaxis. In some embodiments, the method comprises administering inhibitors of GM-CSF, CSF1, GM-CSFR, or CSF1R, lenzilumab, mavrilimumab, cytokines, and/or anti-inflammatory agents.

In some embodiments, the adverse events are managed by the administration of an agent/agents that is/are an antagonist or inhibitor of IL-6 or the IL-6 receptor (IL-6R). In some embodiments, the agent is an antibody that neutralizes IL-6 activity, such as an antibody or antigen-binding fragment that binds to IL-6 or IL-6R. For example, in some embodiments, the agent is or comprises tocilizumab (atlizumab) or sarilumab, anti-IL-6R antibodies. In some embodiments, the agent is an anti-IL-6R antibody described in U.S. Pat. No. 8,562,991. In some cases, the agent that targets IL-6 is an anti-TL-6 antibody, such as siltuximab, elsilimomab, ALD518/BMS-945429, sirukumab (CNTO 136), CPSI-2634, ARGX 109, FE301, FM101, or olokizumab (CDP6038), and combinations thereof. In some embodiments, the agent may neutralize IL-6 activity by inhibiting the ligand-receptor interactions. In some embodiments, the IL-6/IL-6R antagonist or inhibitor is an IL-6 mutein, such as one described in U.S. Pat. No. 5,591,827. In some embodiments, the agent that is an antagonist or inhibitor of IL-6/IL-6R is a small molecule, a protein or peptide, or a nucleic acid.

In some embodiments, other agents that may be used to manage adverse reactions and their symptoms include an antagonist or inhibitor of a cytokine receptor or cytokine. In some embodiments, the cytokine or receptor is IL-10, TL-6, TL-6 receptor, IFNγ, IFNGR, IL-2, IL-2R/CD25, MCP-1, CCR2, CCR4, MIP13, CCRS, TNFalpha, TNFR1, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCRS), TNF alpha receptor (e.g., TNFR1), IL-1 receptor (IL1-Ra/IL-1RP), or IL-10 receptor (IL-10R), IL-1, and IL-1Ralpha/IL-1beta. In some embodiments, the agent comprises situximab, sarilumab, olokizumab (CDP6038), elsilimomab, ALD518/BMS-945429, sirukumab (CNTO 136), CPSI-2634, ARGX 109, FE301, or FM101. In some embodiments, the agent, is an antagonist or inhibitor of a cytokine, such as transforming growth factor beta (TGF-beta), interleukin 6 (TL-6), interleukin 10 (IL-10), IL-2, MIP13 (CCL4), TNF alpha, IL-1, interferon gamma (IFN-gamma), or monocyte chemoattractant protein-I (MCP-1). In some embodiments, the is one that targets (e.g. inhibits or is an antagonist of) a cytokine receptor, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R) and combinations thereof. In some embodiments, the agent is administered by one of the methods and doses described elsewhere in the specification, before, after, or concurrently with the administration of the cells.

In some embodiments, the agent is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the agent is administered in a dosage amount of at least or at least about or about 2 mg/kg, 4 mg/kg, 6 mg/kg or 8 mg/kg. In some embodiments, is administered in a dosage amount from about 1 mg/kg to 12 mg/kg, such as at or about 10 mg/kg. In some embodiments, the agent is administered by intravenous infusion. In one embodiment, the agent is tocilizumab. In some embodiments, the (agent(s), e.g, specifically tocilizumab) is/are administered by one of the methods and doses described elsewhere in the specification, before, after, or concurrently with the administration of the cells.

In some embodiments, the method comprises identifying CRS based on clinical presentation. In some embodiments, the method comprises evaluating for and treating other causes of fever, hypoxia, and hypotension. If CRS is observed or suspected, it may be managed according to the recommendations in protocol A, which may also be used in combination with the other treatments of this disclosure, including Neutralization or Reduction of the CSF/CSFR1 Axis. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered. In some embodiments, a biosimilar or equivalent of tocilizumab may be used instead of tocilizumab in the methods disclosed herein. In other embodiments, another anti-IL6R may be used instead of tocilizumab.

In some embodiments, adverse events are managed according to the following protocol (protocol A/Table 1):

TABLE 3
CRS grading and management guidance

| CRS Grade (a) | Tocilizumab | Corticosteroids |
|---|---|---|
| Grade 1 | If symptoms (e.g., fever) not | If not improving after 3 days, |
| Symptoms require | improving after 24 hours, | administer one dose of |
| symptomatic treatment only | consider managing as Grade | dexamethasone 10 mg |
| (e.g., fever, nausea, fatigue, | 2. | intravenously. |
| headache, myalgia, malaise). | | |
| Grade 2 | Administer tocilizumab c 8 | Administer dexamethasone 10 |
| Symptoms require and | mg/kg intravenously over 1 | mg intravenously once daily. |
| respond to moderate | hour (not to exceed 800 mg). | If improving, manage as |
| intervention. | If no clinical improvement in | Grade 1 above and continue |
| Oxygen requirement less | the signs and symptoms of | corticosteroids until the |
| than 40% FiO₂ or | CRS after the first dose, | severity is Grade 1 or less, |
| hypotension responsive to | repeat tocilizumab every 8 | then quickly taper as clinically |
| fluids or low-dose of one | hours as needed. | appropriate. |
| vasopressor or Grade 2 organ | Limit to a maximum of 3 | If not improving, manage as |
| toxicity (b). | doses in a 24-hour period; | appropriate grade below. |
| | maximum total of 4 doses. | |
| | If improving, discontinue | |
| | tocilizumab. | |
| Grade 3 | Per Grade 2 | Dexamethasone 10 mg |
| Symptoms require and | If improving, manage as | intravenously three times a |
| respond to aggressive | appropriate grade above | day. |
| intervention. | | If improving, manage as |
| Oxygen requirement greater | | appropriate grade above and |
| than or equal to 40% FiO₂ or | | continue corticosteroids until |
| hypotension requiring high- | | the severity is Grade 1 or less, |
| dose or multiple vasopressors | | then quickly taper as clinically |
| or Grade 3 organ toxicity or | | appropriate. |
| Grade 4 transaminitis. | | If not improving, manage as |
| | | Grade 4. |
| Grade 4 | Per Grade 2 | Administer |
| Life-threatening symptoms. | If improving, manage as | intravenously once per day for |
| Requirements for ventilator | appropriate grade above. | 3 days. |
| support, continuous veno- | | methylprednisolone 1000 mg |
| venous hemodialysis | | If improving, manage as |
| (CVVHD) or | | appropriate grade above and |
| Grade 4 organ toxicity | | continue corticosteroids until |
| (excluding transaminitis). | | the severity is Grade 1 or less, |
| | | then taper as clinically |
| | | appropriate. |
| | | If not improving, consider |
| | | methylprednisolone 1000 mg |
| | | 2-3 times a day or alternate |
| | | therapy.d |

(a) Lee DW et al., (2014). Current concepts in the diagnosis and management of cytokine release syndrome. Blood. 2014 Jul. 10; 124(2): 188-195.
(b) Refer to Procotocol B for management of neurologic toxicity.
c Refer to ACEMTRA ® (tocilizumab) Prescribing Information for details, https://www.gene.com/download/pdf/actemra_prescribing.pdf (last accessed Oct. 18, 2017). Initial U.S. approval is indicated to be in 2010.
dAlternate therapy includes (but is not limited to): anakinra, siltuximab, ruxolitinib, cyclophosphamide, IVIG and ATG.

### Neurologic Toxicity

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. Consider non-sedating, anti-seizure medicines (e.g., levetiracetam) for seizure prophylaxis for any ≥Grade 2 neurologic toxicities. The following treatments may be used in combination with the other treatments of this disclosure, including Neutralization or Reduction of the CSF/CSFR1 Axis.

In some embodiments, NE are managed according to the following protocol (protocol B/Table 4):

TABLE 4
Neurologic toxicity grading and management guidance

| Grading | | |
|---|---|---|
| Assessment | Concurrent CRS | No concurrent CRS |
| Grade 1 | Administer tocilizumab per protocol A | Administer one dose of |
| | for management of Grade 1 CRS. | dexamethasone 10 mg |
| | In addition, administer one dose of | intravenously. |
| | dexamethasone 10 mg intravenously. | If not improving after 2 |
| | If not improving after 2 days, repeat | days, repeat dexamethasone |
| | dexamethasone 10 mg intravenously. | 10 mg intravenously. |
| | Consider levetiracetam for seizure | Consider levetiracetam for |
| | prophylax | seizure prophylax |
| Grade 2 | Administer tocilizumab per Table 1 for | Administer dexamethasone |
| | management of Grade 2 CRS. | 10 mg intravenously four |
| | In addition, administer dexamethasone 10 | times a day |
| | mg intravenously four times a day. | If improving, continue |
| | If improving, continue corticosteroids | corticosteroids until the |
| | until the severity is Grade 1 or less, then | severity is Grade 1 or less, |
| | quickly taper as clinically appropriate. | then quickly taper as |
| | If not improving, manage as appropriate | clinically appropriate. |
| | grade below. | If not improving, manage as |
| | | appropriate grade below. |
| | Consider non-sedating, anti-seizure medicines (e.g., | |
| | levetiracetam) for seizure prophylaxis. | |
| Grade 3 | Administer tocilizumab per (protocol A) | Administer |
| | for management of Grade 2 CRS. | methylprednisolone 1000 |
| | In addition, administer | mg intravenously once daily. |
| | methylprednisolone 1000 mg | If improving, manage as |
| | intravenously once daily. | appropriate grade above and |
| | If improving, manage as appropriate | continue corticosteroids |
| | grade above and continue corticosteroids | until the severity is Grade 1 |
| | until the severity is Grade 1 or less, then | or less, then taper as |
| | taper as clinically appropriate. | clinically appropriate. |
| | If not improving, manage as Grade 4. | If not improving, manage as |
| | | Grade 4. |
| | Consider non-sedating, anti-seizure medicines (e.g., | |
| | levetiracetam) for seizure prophylaxis. | |
| Grade 4 | Administer tocilizumab per Table 1 for | Administer |
| | management of Grade 2 CRS. | methylprednisolone 1000 |
| | In addition, administer | mg intravenously twice per |
| | methylprednisolone 1000 mg | day |
| | intravenously twice per day. | If improving, manage as |
| | If improving, manage as appropriate | appropriate grade above and |
| | grade above and continue corticosteroids | continue corticosteroids |
| | until the severity is Grade 1 or less, then | until the severity is Grade 1 |
| | taper as clinically appropriate. | or less, then taper as |
| | If not improving, consider 1000 mg of | clinically appropriate. |
| | methylprednisolone intravenously 3 times | If not improving, consider |
| | a day or alternate therapy.b | 1000 mg of methylprednisolone |
| | | intravenously 3 times a day |
| | | or alternate therapy.b |

Consider non-sedating, anti-seizure medicines (e.g.,
levetiracetam) for seizure prophylaxis.
^{a.}Severity based on Common Terminology Criteria for Adverse Events.
bAlternate therapy includes (but is not limited to): anakinra, siltuximab, ruxolitinib, cyclophosphamide, IVIG and ATG.
methylprednisolone may be substituted for equivalent levels of dexamethasone.

Additional Safety Management Strategies with Corticosteroids

Administration of corticosteroids and/or tocilizumab at Grade 1 may be considered prophylactic. Supportive care may be provided in all protocols at all CRS and NE severity grades.

In one embodiment of a protocol for management of adverse events related to CRS, tocilizumab and/or corticosteroids are administered as follows: Grade 1 CRS: no tocilizumab; no corticosteroids; Grade 2 CRS: tocilizumab (only in case of comorbidities or older age); and/or corticosteroids (only in case of comorbidities or older age); Grade 3 CRS: tocilizumab; and/or corticosteroids; Grade 4 CRS: tocilizumab; and/or corticosteroids. In another embodiment of a protocol for management of adverse events related to CRS, tocilizumab and/or corticosteroids are administered as follows: Grade 1 CRS: tocilizumab (if no improvement after 3 days); and/or corticosteroids (if no improvement after 3 days); Grade 2 CRS: tocilizumab; and/or corticosteroids; Grade 3 CRS: tocilizumab; and/or corticosteroids; Grade 4 CRS: tocilizumab; and/or corticosteroids, high dose.

In one embodiment of a protocol for management of adverse events related to NE, tocilizumab and/or corticosteroids are administered as follows: Grade 1 NE: no tocilizumab; no corticosteroids;

Grade 2 NE: no tocilizumab; no corticosteroids; Grade 3 NE: tocilizumab; and/or corticosteroids (only if no improvement to tocilizumab, standard dose); Grade 4 NE: tocilizumab; and/or corticosteroids.

In another embodiment of a protocol for management of adverse events related to NE, tocilizumab and/or corticosteroids are administered as follows: Grade 1 NE: no tocilizumab; and/or corticosteroids; Grade 2 NE: tocilizumab; and/or corticosteroids; Grade 3 NE: tocilizumab; and/or corticosteroids, high dose; Grade 4 NE: tocilizumab; and/or corticosteroids, high dose.

In one embodiment, corticosteroid treatment is initiated at CRS grade ≥2 and tocilizumab is initiated at CRS grade ≥2. In one embodiment, corticosteroid treatment is initiated at CRS grade ≥1 and tocilizumab is initiated at CRS grade ≥1. In one embodiment, corticosteroid treatment is initiated at NE grade ≥3 and tocilizumab is initiated at CRS grade ≥3. In one embodiment, corticosteroid treatment is initiated at CRS grade ≥1 and tocilizumab is initiated at CRS grade ≥2. In some embodiments, prophylactic use of tocilizumab administered on Day 2 may decrease the rates of Grade ≥3 CRS.

In one embodiment, the protocol for treatment of adverse events comprises Protocol C, as follows (Table 5)

TABLE 5
Alternative adverse management guidance

| CRS | | |
|---|---|---|
| Grade | Tocilizumab Dose^{a} | Corticosteroid Dose^{a} |
| 1 | 8 mg/kg over 1 hour^{b} if no | Dexamethasone 10 mg × 1 |
| | improvement after 24 hours of | if no improvement after 3 days |
| | supportive care; repeat every | |
| | 4-6 hours as needed | |
| 2 | 8 mg/kg over 1 hour^{b}; repeat | Dexamethasone 10 mg × 1 |
| | every 4-6 hours as needed | |
| 3 | Per Grade 2 | Methylprednisolone 1 mg/kg IV |
| | | twice daily or equivalent |
| | | dexamethasone dose |
| 4 | Per Grade 2 | Methylprednisolone 1000 mg/d |
| | | IV for 3 days |
| NE | | |
| Grade | Tocilizumab Dose | Corticosteroid Dose |
| 1 | N/A | Dexamethasone 10 mg × 1 |
| 2 | Only in the case of concurrent | Dexamethasone 10 mg 4×/day |
| | CRS; 8 mg/kg over 1 hour; | |
| | repeat every 4-6 hours as | |
| | needed | |
| 3 | Per Grade 2 | Methylprednisolone 1 g once |
| | | daily |
| 4 | Per Grade 2 | Methylprednisolone 1 g twice |
| | | daily |

^{a}Therapy to be tapered on improvement of symptoms at investigator's discretion;
^{b}Not to exceed 800 mg;
AE, adverse event;
CRS, cytokine release syndrome;
IV, intravenous;
N/A, not applicable;
NE, neurologic event

Any corticosteroid may be appropriate for this use. In one embodiment, the corticosteroid is dexamethasone. In some embodiments, the corticosteroid is methylprednisolone. In some embodiments, the two are administered in combination. In some embodiments, glucocorticoids include synthetic and non-synthetic glucocorticoids. Exemplary glucocorticoids include, but are not limited to: alclomethasones, algestones, beclomethasones (e.g. beclomethasone dipropionate), betamethasones (e.g. betamethasone 17 valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, flupredni solones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylpredni solones (methylprednisolone aceponate, methylprednisolone acetate, methylprednisolone hemi succinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone famesylate, prednisolone hemi succinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21 palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980) and Remington: The Science and Practice of Pharmacy, 22nd Edition, Lippincott Williams & Wilkins, Philadelphia, Pa. (2013) and any other editions, which are hereby incorporated by reference. In some embodiments, the glucocorticoid is selected from among cortisones, dexamethasones, hydrocortisones, methylprednisolones, prednisolones and prednisones. In an embodiment, the glucocorticoid is dexamethasone. In other embodiments, the steroid is a mineralcorticoid. Any other steroid may be used in the methods provided herein.

The one or more corticosteroids may be administered at any dose and frequency of administration, which may be adjusted to the severity/grade of the adverse event (e.g., CRS and NE). The tables above provide examples of dosage regimens for management of CRS and NE, respectively. In another embodiment, corticosteroid administration comprises oral or IV dexamethasone 10 mg, 1-4 times per day. Another embodiment, sometimes referred to as “high-dose” corticosteroids, comprises administration of IV methylprednisone 1 g per day alone, or in combination with dexamethasone. In some embodiments, the one or more cortico steroids are administered at doses of 1-2 mg/kg per day.

The corticosteroid may be administered in any amount that is effective to ameliorate one or more symptoms associated with the adverse events, such as with the CRS or neurotoxicity. The corticosteroid, e.g., glucocorticoid, may be administered, for example, at an amount between at or about 0.1 and 100 mg, per dose, 0.1 to 80 mg, 0.1 to 60 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject. Typically, the corticosteroid, such as a glucocorticoid is administered at an amount between at or about 0.4 and 20 mg, for example, at or about 0.4 mg, 0.5 mg, 0.6 mg, 0.7 mg, 0.75 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg or 20 mg per dose, to an average adult human subject.

In some embodiments, the corticosteroid may be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

Generally, the dose of corticosteroid administered is dependent upon the specific corticosteroid, as a difference in potency exists between different corticosteroids. It is typically understood that drugs vary in potency, and that doses may therefore vary, in order to obtain equivalent effects. Equivalence in terms of potency for various glucocorticoids and routes of administration. is well known. Information relating to equivalent steroid dosing (in a non-chronotherapeutic manner) may be found in the British National Formulary (BNF) 37, March 1999.

In some embodiments, the adverse events are managed by the following protocol: patients receive levetiracetam (750 mg oral or intravenous twice daily) starting on day 0 of administration of T cell therapy; at the onset of grade ≥2 neurologic events, levetiracetam dose is increased to 1000 mg twice daily; if a patient did not experience any grade ≥2 neurologic event, levetiracetam is tapered and discontinued as clinically indicated; patients also receive tocilizumab (8 mg/kg IV over 1 hour [not to exceed 800 mg]) on day 2; further tocilizumab (±corticosteroids) may be recommended at the onset of grade 2 CRS in patients with comorbidities or older age, or otherwise in case of grade ≥3 CRS; for patients experiencing grade ≥2 neurologic events, tocilizumab is initiated, and corticosteroids are added for patients with comorbidities or older age, or if there is any occurrence of a grade ≥3 neurologic event with worsening symptoms despite tocilizumab use. In some embodiments, levetiracetam is administered for prophylaxis and at the onset of grade ≥2 neurologic toxicities, if neurologic events occur after the discontinuation of prophylactic levetiracetam and/or levetiracetam is tapered and discontinued if the patient does not experience any grade ≥2 neurologic toxicities.

In some embodiments, the adverse events are managed by the following protocol: patients receive dexamethasone 10 mg PO on Days 0 (prior to T cell therapy infusion), 1, and 2; steroids are also administered starting at Grade 1 NE, and for Grade 1 CRS when no improvement is observed after 3 days of supportive care; tocilizumab is also administered for Grade ≥1 CRS if no improvement is observed after 24 hours of supportive care.

### Secondary Malignancies

In some embodiments, patients treated with CAR T cells (e.g., CD19-directed) or other genetically modified autologous T cell immunotherapy may develop secondary malignancies. In certain embodiments, patients treated with CAR T cells (.e.g, CD19-directed) or other genetically modified allogeneic T cell immunotherapy may develop secondary malignancies. In some embodiments, the method comprises monitoring life-long for secondary malignancies.

Methods and Compositions to Generate a Product for Increased Clinical Efficacy and/or Decreased Toxicity

In one embodiment, the disclosure provides a method of manufacturing an immunotherapy product with improved clinical efficacy and/or decreased toxicity to be used in patients according to the predicted grade of toxicity (CRS/NE). In some embodiments, the immunotherapy product comprises blood cells. In some embodiments, blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some embodiments, a washing step is accomplished a semi-automated“flow-through” centrifuge (for example, the Cobe 2991 cell processor, Baxter) according to the manufacturer's instructions. In some embodiments, a washing step is accomplished by tangential flow filtration (TFF) according to the manufacturer's instructions. In some embodiments, the cells are resuspended in a variety of biocompatible buffers after washing, such as, for example, Ca++Mg++free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient. In some embodiments, the methods include leukapheresis.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some embodiments includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some embodiments of such processes, a volume of cells is mixed with an amount of a desired affinity-based selection reagent. The immunoaffinity-based selection may be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) may be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a chamber, for example, under centrifugal rotation. In some embodiments, incubation of cells with selection reagents, such as immunoaffinity-based selection reagents, is performed in a chamber.

In some embodiments, by conducting such selection steps or portions thereof (e.g., incubation with antibody-coated particles, e.g., magnetic beads) in the cavity of a chamber, the user is able to control certain parameters, such as volume of various solutions, addition of solution during processing and timing thereof, which may provide advantages compared to other available methods. For example, the ability to decrease the liquid volume in the cavity during the incubation may increase the concentration of the particles (e.g. bead reagent) used in the selection, and thus the chemical potential of the solution, without affecting the total number of cells in the cavity. This in turn may enhance the pairwise interactions between the cells being processed and the particles used for selection.

In some embodiments, carrying out the incubation step in the chamber, e.g., when associated with the systems, circuitry, and control as described herein, permits the user to effect agitation of the solution at desired time(s) during the incubation, which also may improve the interaction.

In some embodiments, at least a portion of the selection step is performed in a chamber, which includes incubation of cells with a selection reagent. In some embodiments of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which may aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some embodiments also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps may be based on positive selection, in which the cells having bound the reagents are retained for further use, and/or negative selection, in which the cells having not bound to the antibody or binding partner are retained. In some examples, both fractions are retained for further use.

In some embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+, CD28+ T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations may be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long term survival, expansion, and/or engraftment following administration, which in some embodiments is particularly robust in such sub-populations. In some embodiments, combining TcM-enriched CD8+ T cells and CD4+ T cells further enhances efficacy. In some embodiments, enriching for T cells with naïve phenotype (CD45RA+CCR7+) enhances efficacy.

In embodiments, memory T cells are present in both CD62L+ and CD62L subsets of CD8+peripheral blood lymphocytes. PBMC may be enriched for or depleted of CD62L CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some embodiments, it is based on negative selection for cells expressing or highly expressing CD45RA and/or granzyme B. In some embodiments, isolation of a CD8+ population enriched for TCM cells is carried out by depletion of cells expressing CD4, CD 14, CD45RA, and positive selection or enrichment for cells expressing CD62L. In one embodiment, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some embodiments are carried out simultaneously and in other embodiments are carried out sequentially, in either order. In some embodiments, the same CD4 expression-based selection step used in preparing the CD8+cell population or subpopulation, also is used to generate the CD4+cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naive, central memory, and effector cells by identifying cell populations that have cell surface antigens. CD4+ lymphocytes may be obtained by standard methods. In some embodiments, naive CD4+ T lymphocytes are CD45RO, CD45RA+, CD62L+, CD4+ T cells. In some embodiments, central memory CD4+ cells are CD62L+ and CD45RO+. In some embodiments, effector CD4+ cells are CD62L and CD45RO. In some embodiments, T cells with naïve phenotype are CD45RA+CCR7+.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques.

In some embodiments, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps.

In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., ah, of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps.

In some embodiments, the separation and/or other steps is carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls ah components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some embodiments is equipped with a cell processing unity that permits automated washing and fractionation of cells by centrifugation. The CliniMACS Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some embodiments may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In one embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the cells are incubated and/or cultured prior to or in connection with genetic engineering. The incubation steps may include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions may include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some embodiments, the agent turns on or initiates TCR/CD3 intracellular signaling cascade in a T cell. Such agents may include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some embodiments, the IL-2 concentration is at least about 10 units/mL. In some embodiments, incubation is carried out in accordance with techniques such as those described in U.S. Pat. No. 6,040,177 to Riddell et al., Klebanoff et al. (2012) J Immunother. 35(9): 651— 660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some embodiments, the non-dividing feeder cells may comprise gamma-irradiated PBMC feeder cells. In some embodiments, the PBMC are irradiated with gamma rays in the range of about 3000 to 3600 rads to prevent cell division. In some embodiments, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL may be irradiated with gamma rays in the range of about 6000 to 10,000 rads. The LCL feeder cells in some embodiments is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones may be generated to cytomegalovirus antigens by isolating T cells from infected subjects and stimulating the cells in vitro with the same antigen.

In some embodiments, at least a portion of the incubation in the presence of one or more stimulating conditions or stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some embodiments of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which may aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80g to 100g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the stimulation results in activation and/or proliferation of the cells, for example, prior to transduction.

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g. retroviral or lentiviral, transduction, transposons, and electroporation. In some embodiments, the engineering produces one or more engineered compositions of enriched T cells.

In certain embodiments, the one or more compositions of stimulated T cells are or include two separate stimulated compositions of enriched T cells. In some embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In some embodiments, the two separate compositions include a composition of enriched CD8+ T cells. In some embodiments, two separate compositions of enriched CD4+ T cells and enriched CD8+ T cells are genetically engineered separately. In some embodiments, the same composition is enriched for both CD4+ T cells and CD8+ T cells and these are genetically engineered together.

In one embodiment, the sample of T lymphocytes is prepared by leukapheresis of PBMCs from the subject. In one embodiment, the leukapheresis sample is further subject to T lymphocyte enrichment through positive selection for CD4+ and/or CD8+ cells. In one embodiment, the lymphocytes are further engineered to comprise a CAR or an exogenous TCR. Examples of CARs and TCRs and methods of engineering lymphocytes are described elsewhere in the disclosure. In one embodiment, the method comprises expanding the engineered lymphocytes to produce a T cell infusion product in the presence of IL-2. In one embodiment, the engineered lymphocytes are expanded for about 2-7 days in the presence of IL-2.

Under circumstances where subjects initially respond and subsequently relapse, subjects may be eligible for a second course of conditioning chemotherapy and axicabtagene ciloleucel. Retreatment may be administered under conditions such as: subject has a PR or CR; subject's disease subsequently progresses; CD19 tumor expression confirmed locally by biopsy after disease progression and prior to re-treatment; Subject continues to meet the original study eligibility criteria with exception of prior axicabtagene ciloleucel use. Screening assessments should be repeated if clinically indicated, as determined by the investigator, to confirm eligibility; Subject has not received subsequent therapy for the treatment of lymphoma; Toxicities related to conditioning chemotherapy (fludarabine and cyclophosphamide), with the exception of alopecia, have resolved to ≤Grade 1 or returned to baseline prior to retreatment; and Subject does not have known neutralizing antibodies (exception: if a non-neutralizing antibody develops subject may be retreated if they meet the original study eligibility criteria).

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference. All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

The following examples are intended to illustrate various aspects of the application. As such, the specific aspects discussed are not to be construed as limitations on the scope of the application. For example, although the Examples below are directed to T cells transduced with an anti-CD19 chimeric antigen receptor (CAR), one skilled in the art would understand that the methods described herein may apply to immune cells transduced with any CAR. The methods are also applicable to other immunotherapies. It will be apparent to one skilled in the art that various equivalents, changes, and modifications may be made without departing from the scope of application, and it is understood that such equivalent aspects are to be included herein.

The disclosures provided by this application may be used in a variety of methods in additional to, or as a combination of, the methods described above. The following is a compilation of exemplary methods that may be derived from the disclosures provided in this application.

### EXAMPLES

### Example 1

ZUMA-1 (NCT02348216) is a clinical study wherein patients with relapsed/refractory NHL have been treated with axicabtagene ciloleucel. Axicabtagene ciloleucel is a CD19-directed genetically modified autologous T cell immunotherapy, comprising the patient's own T cells harvested and genetically modified ex vivo by retroviral transduction to express a chimeric antigen receptor (CAR) comprising an anti-CD19 single chain variable fragment (scFv) linked to CD28 and CD3-zeta co-stimulatory domains. Patients may have had diffuse large B-cell lymphoma, primary mediastinal B-cell lymphoma, or transformed follicular lymphoma with refractory disease despite undergoing recommended prior therapy. Patients received a target dose of 2×10⁶ anti-CD19 CAR T cells per kilogram of body weight after receiving a conditioning regimen of low-dose cyclophosphamide and fludarabine. (Neelapu, S S et al. 2017, N Engl J Med 2017; 377(26):2531-44. Safety and efficacy results were previously reported. (Neelapu, S S et al. 2017, N Engl J Med 2017; 377(26):2531-44; Locke F L et al. 2019; Lancet Oncol. 2019 January; 20(1):31-42. doi:10.1016/51470-2045(18)30864-7. Epub 2018 December 2).

Available samples from patients in ZUMA-1 (NCT02348216) were analyzed. Cohorts 1 and 2 represent the pivotal cohorts. (Locke F L, et al. Lancet Oncol. 2019; 20:31-42; Neelapu S S, et al. N Engl J Med. 2017; 377:2531-2544). In a 2-year analysis (median follow-up, 27.1 months; N=101), axicabtagene ciloleucel treatment showed objective response, complete response, and ongoing response rates of 83%, 58%, and 39%, respectively. After a median of 39.1 months of follow-up, median overall survival (OS) was 25.8 months, with a 3-year OS rate of 47%. Cytokine release syndrome (CRS) and neurologic events are common in patients receiving anti-CD19 CAR T-cell therapies and may be severe or life-threatening. At the 2-year follow-up of the 108 patients in phases 1 and 2 of ZUMA-1 (data cutoff, Aug. 11, 2018), grade ≥3 CRS was reported in 11% of patients and grade ≥3 neurologic events were reported in 32%. The majority of CRS cases and neurologic events were manageable and reversible

To reduce axicabtagene ciloleucel—related toxicity, several exploratory safety management cohorts were added to ZUMA-1 in refractory LBCL. Cohort 4 evaluated the rates and severity of cytokine release syndrome (CRS) and neurologic events (NEs) with earlier corticosteroid and tocilizumab use. 7Primary endpoints were incidence and severity of CRS and NEs. In Cohort 4, forty-one patients received axicabtagene ciloleucel. Incidences of any-grade CRS and NEs were 93% and 61%, respectively (grade ≥3, 2% and 17%, respectively). There was no grade 4 or 5 CRS or NE. Despite earlier dosing, the cumulative cortisone-equivalent corticosteroid dose in patients requiring corticosteroid therapy was lower than that reported in the pivotal ZUMA-1 cohorts. With a median follow-up of 14.8 months, objective and complete response rates were 73% and 51%, respectively, and 51% of treated patients were in ongoing response. Earlier and measured use of corticosteroids and/or tocilizumab has the potential to reduce the incidence of grade ≥3 CRS and NEs in patients with R/R LBCL receiving axicabtagene ciloleucel.

Eligible patients in cohort 4 had R/R LBCL after ≥2 systemic lines of therapy or were refractory to first-line therapy (i.e., best response of progressive disease (PD) or stable disease (to ≥4 cycles of first-line therapy with stable disease duration no longer than 6 months). Prior therapy must have included an anti-CD20 monoclonal antibody (unless the tumour was CD20-negative) and an anthracycline-containing chemotherapy regimen. Patients were required to have an Eastern Cooperative Oncology Group performance status of 0 or 1. Additional inclusion criteria were absolute neutrophil count >1,000 cells/μL, absolute lymphocyte count >100 cells/μL, platelet count >75,000 cells/μL, adequate organ function, no central nervous system involvement, and no active infection.

Cohort 4 patients received a conditioning regimen of cyclophosphamide (500 mg/m²/day) and fludarabine (30 mg/m²/day) on days −5 to −3, and 1 dose of axicabtagene ciloleucel (target dose, 2×10⁶ CAR T cells/kg) on day 0. Bridging therapy prior to initiation of conditioning chemotherapy was allowed per investigator's discretion (e.g., bulky disease or rapidly progressing disease at screening or baseline).

Baseline characteristics for Cohort 4 were generally comparable to those of Cohorts 1+2, with the exceptions of lower pre-treatment tumor burden, lower levels of inflammatory markers (eg, ferritin, lactate dehydrogenase [LDH]), and a lower proportion of patients with progressive disease in response to the most recent line of therapy in Cohort 4 (Table 6). Unlike Cohorts 1+2 in which patients had to be refractory after second-line or later therapy, patients in Cohort 4 could be relapsed or refractory. Consequently, there were 12% who were relapsed after second-line or later therapy (0 in Cohorts 1+2).

TABLE 6
Baseline Characteristics by Cohort

| | Cohorts 1 + 2 | | Cohort 4 | |
|---|---|---|---|---|
| Characteristic | (N = 101) | | (N = 41) | |
| Disease type, n (%) | | | | |
| DLBCL | 77 | (76) | 26 | (63) |
| PMBCL | 8 | (8) | 2 | (5) |
| TFL | 16 | (16) | 10 | (24) |
| HGBCL | NA^{a} | | 3 | (7) |
| Age | | | | |
| Median (range), years-old | 58 | (23-76) | 61.0 | (19-77) |
| ≥65 years-old, n (%) | 24 | (24) | 13 | (32) |
| Male sex, n (%) | 68 | (67) | 28 | (68) |
| ECOG performance status | 59 | (58) | 20 | (49) |
| score of 1, n (%) | | | | |
| Disease stage, n (%) | | | | |
| I or II | 15 | (15) | 11 | (27) |
| III or IV | 86 | (85) | 29 | (71) |
| IPI score, n (%) | | | | |
| 0-2 | 55 | (54) | 21 | (51) |
| 3-4 | 46 | (46) | 20 | (49) |
| CD19 positivity, n/N (%) | | | | |
| Yes | 74/82 | (90) | 22/24 | (92) |
| No | 8/82 | (10) | 2/24 | (8) |
| Number of prior lines of | | | | |
| chemotherapy, % | | | | |
| 1 | 3 | (3) | 0 | |
| 2 | 28 | (28) | 15 | (37) |
| 3 | 29 | (29) | 15 | (37) |
| 4 | 29 | (29) | 8 | (20) |
| ≥5 | 12 | (12) | 3 | (7) |
| Prior SCT, n (%) | 25 | (25) | 14 | (34) |
| PD as best response to most | 67 | (66) | 15 | (37) |
| recent chemotherapy^{b}, n (%) | | | | |
| Median tumor burden | 3721 | (171-23,297) | 2100 | (204-24,758) |
| by SPD^{c} (range), mm² | | | | |
| Median LDH, U/L (range) | 344 | (116-7802) | 262 | (145-4735) |
| Median ferritin, ng/ml (range) | 777 | (1-10,576) | 393 | (23-3457) |
| Refractory subgroup, n (%) | | | | |
| Primary refractory | 3 | (3) | 0 | (0) |
| Refractory ≥ 2^{nd}-line therapy | 77 | (76) | 28 | (68) |
| Relapsed ≥ 2^{nd}-line therapy | NA | | 5 | (12) |
| Relapsed post-ASCT | 21 | (21) | 8 | (20) |

^{a}HGBCL was captured as DLBCL in Cohorts 1 + 2. Pretreatment tumor samples were assessed in 47 patients; 7 (15%) had HGBCL.20
^{b}For patients who had not relapsed post-ASCT.
^{c}Measured after bridging.
ASCT, autologous stem cell transplant; DLBCL, diffuse large B-cell lymphoma; ECOG, Eastern Cooperative Oncology Group; HGBCL, high grade B-cell lymphoma; IPI, International Prognostic Index; LDH, lactate dehydrogenase; NA, not applicable; PMBCL, primary mediastinal B-cell lymphoma; SPD, sum of the products of diameters; TFL, transformed follicular lymphoma.

TABLE 7
Tocilizumab and corticosteroid guidelines for
adverse event management in ZUMA-1 cohort 4.

| CRS | | |
|---|---|---|
| grade | Tocilizumab dose* | Corticosteroid dose* |
| 1 | If no improvement after 3 days, | If no improvement after 3 days, |
| | 8 mg/kg over 1 hour^{†}; repeat | dexamethasone 10 mg × 1 |
| | every 4-6 hours as needed | |
| 2 | 8 mg/kg over 1 hour^{†}; repeat | Dexamethasone 10 mg × 1 |
| | every 4-6 hours as needed | |
| 3 | Per grade 2 | Methylprednisolone 1 mg/kg IV |
| | | twice daily or equivalent |
| | | dexamethasone dose |
| 4 | Per grade 2 | Methylprednisolone 1000 |
| | | mg/day IV × 3 days |
| NE | | |
| grade | Tocilizumab dose | Corticosteroid dose |
| 1 | N/A | Dexamethasone 10 mg × 1 |
| 2 | 8 mg/kg over 1 hour; repeat | Dexamethasone 10 mg 4 |
| | every 4-6 hours as needed | times/day |
| 3 | As per grade 2 | Methylprednisolone 1 g |
| | | once daily |
| 4 | As per grade 2 | Methylprednisolone 1 g |
| | | twice daily |

CRS, cytokine release syndrome; IV, intravenously; N/A, not applicable; NE, neurologic event.
*Therapy to be tapered upon improvement of symptoms at investigator's discretion.
^{†}Not to exceed 800 mg.

The primary endpoint in cohort 4 was the incidence and severity of CRS and NEs. CRS was graded according to modified Lee et al criteria (Lee et al, Blood. 2014, 124(2): 188-195) and NEs were graded per Common Terminology Criteria for Adverse Events version 4.03. Key safety-related secondary endpoints included the incidence of other adverse events and clinically significant changes in safety laboratory values. Key efficacy-related secondary endpoints included ORR per investigator assessment, duration of response, PFS, OS, anti-CD19 CAR T-cell levels in the blood, and cytokine levels in the serum.

TABLE 8
Comparison of efficacy and safety outcomes and CAR T-cell
and soluble serum biomarker levels between patients in cohorts
1 + 2 and cohort 4 before and after propensity score matching.

| | Cohorts 1 + 2 | | Cohorts 1 + 2 | | | |
|---|---|---|---|---|---|---|
| | overall | | after matching | | Cohort 4 | |
| Characteristic | (N = 101) | | (N = 41) | | (N = 41) | |
| Efficacy | | | | | | |
| Response | | | | | | |
| Objective | 84 | (83.2) | 38 | (92.7) | 30 | (73.2) |
| response, n (%) | | | | | | |
| Complete | 59 | (58.4) | 31 | (75.6) | 21 | (51.2) |
| response, n (%) | | | | | | |
| Ongoing response | 42 | (41.6) | 21 | (51.2) | 21 | (51.2) |
| at data cutoff, n | | | | | | |
| (%) | | | | | | |
| Safety | | | | | | |
| CRS | | | | | | |
| Worst grade 2 | 45 | (44.6) | 16 | (39.0) | 24 | (58.5) |
| Worst grade ≥3, | 12 | (11.9) | 6 | (14.6) | 1 | (2.4) |
| n (%) | | | | | | |
| Median (Q1-Q3) | 2 | (2-3) | 2 | (2-3) | 2 | (1-4) |
| time to onset of | | | | | | |
| any grade CRS, | | | | | | |
| days | | | | | | |
| NEs | | | | | | |
| Worst grade 2 | 14 | (13.9) | 5 | (12.2) | 4 | (9.8) |
| Worst grade ≥3, | 29 | (28.7) | 11 | (26.8) | 7 | (17.1) |
| n (%) | | | | | | |
| Median (Q1-Q3) | 5 | (3-7) | 6 | (3-7) | 6 | (2-9) |
| time to onset of | | | | | | |
| any grade NE, | | | | | | |
| days | | | | | | |
| Corticosteroid | | | | | | |
| use* | | | | | | |
| Patients receiving | 26 | (25.7) | 8 | (19.5) | 30 | (73.2) |
| corticosteroids, | | | | | | |
| n (%) | | | | | | |
| Median (Q1-Q3) | 6387 (3051- | | 6886 (1565- | | 939 (626- | |
| cumulative | 15,862) | | 15,963) | | 8138) | |
| corticosteroid | | | | | | |
| dose, mg | | | | | | |
| Tocilizumab use | | | | | | |
| Patients receiving | 43 | (42.6) | 12 | (29.3) | 31 | (75.6) |
| tocilizumab, | | | | | | |
| n (%) | | | | | | |
| Pharmacokinetics | | | | | | |
| and pharma- | | | | | | |
| codynamics | | | | | | |
| Peak CAR T-cell | | | | | | |
| levels, median | | | | | | |
| (Q1-Q3) | | | | | | |
| CAR T-cell | 38.3 (14.7- | | 33.8 (17.1- | | 52.9 (27.3- | |
| expansion, | 83.0) | | 106.9) | | 92.8) | |
| cells/μl | | | | | | |
| AUC₀₋₂₈, cells/ | 453.5 (148.7- | | 450.0 (231.9- | | 511.2 (216.0- | |
| μl × day | 920.3) | | 975.6) | | 973.5) | |
| Peak cytokine | | | | | | |
| levels, median | | | | | | |
| (Q1-Q3) | | | | | | |

| | | | |
|---|---|---|---|
| IFN-γ, pg/ml | 477.4 (196.3- | 452.0 (137.3- | 334.5 (136.1- |
| | 1096.7) | 1094.3) | 737.3) |
| IL-15, pg/ml | 52.9 (34.7- | 56.5 (36.1- | 45.8 (31.2- |
| | 72.1) | 74.4) | 59.5) |
| IL-2, pg/ml | 21.7 (10.2- | 29.7 (10.2- | 11.2 (5.2- |
| | 37.8) | 45.9) | 20.9) |
| IL-6, pg/ml | 83.3 (23.3- | 63.90 (15.9- | 136.70 (14.9- |
| | 347.5) | 261.0) | 366.3) |
| IL-8, pg/ml | 93.6 (46.6- | 124.9 (37.0- | 67.4 (31.6- |
| | 329.3) | 329.9) | 175.2) |
| MCP-1 (CCL2), | 1500.0 (900.1- | 1500.0 (879.5- | 1221.8 (748.9- |
| pg/ml | 1500.0) | 1500.0) | 1500.0) |
| CRP, mg/l | 214.2 (141.4- | 185.2 (141.4- | 126.5 (60.9- |
| | 353.4) | 382.1) | 275.6) |
| Ferritin, ng/ml | 3001.4 (1325.6- | 2461.1 (1154.9- | 1086.4 (481.0- |
| | 6683.5) | 5819.1) | 1586.6) |
| GM-CSF, pg/ml | 7.3 (1.9- | 9.5 (1.9- | 4.4 (1.9- |
| | 16.1) | 22.5) | 6.9) |

AUC₀₋₂₈, area under the curve from day 0 to day 28; CAR, chimeric antigen receptor; CRP, C-reactive protein; CRS, cytokine release syndrome; GM-CSF, granulocyte-macrophage colony-stimulating factor; IFN, interferon; IL, interleukin; MCP-1, monocyte chemoattractant protein-1; NE, neurologic event; Q, quartile.
*Corticosteroid use includes those doses that started on or after the start date of axicabtagene ciloleucel but before the hospital discharge date

Although the overall incidence of CRS in Cohort 4 was comparable to that of Cohorts 1+2 (both 93%), the incidence of grade ≥3 CRS was 2% in Cohort 4 and 12% in Cohorts 1+2 (Table 19); incidence, severity, onset, and duration of CRS and neurologic Events). In Cohort 4, no grade 4 CRS events occurred while in Cohorts 1+2, grade 4 CRS occurred in 3% of patients. CRS resolved by the data cutoff date in all patients in Cohort 4 and 93 of 94 patients in Cohorts 1+2. There were no deaths in the setting of CRS in Cohort 4. In Cohorts 1+2, one patient died from hemophagocytic lymphohistiocytosis and one grade 5 cardiac arrest occurred in a patient with CRS. Time to onset and duration of CRS were similar across cohorts.

A similar pattern was observed with neurologic events: the overall incidence was comparable in Cohort 4 and Cohorts 1+2 (61% vs 64%, respectively), with incidences of grade ≥3 neurologic events of 17% and 29% (Table 9). In Cohort 4, no grade 4 neurologic events were observed while 2% of patients in Cohorts 1+2 experienced grade 4 neurologic events. There were no grade 5 neurologic events in either cohort. Neurologic events resolved in 20 of 25 patients in Cohort 4 and 60 of 64 patients in Cohorts 1+2. Time to onset and duration of neurologic events were both similar across cohorts. All grade ≥3 neurologic events in Cohort 4 occurred in patients who received bridging therapy, possibly due to selection of patients with more aggressive disease in this group.

TABLE 9
Overall incidence of adverse events by Cohort

| | | |
|---|---|---|
| | Cohorts 1 + 2 | Cohort 4 |
| TEAE | (N = 101) | (N = 41) |

CRS

| Any, n (%) | 94 | (93) | 38 | (93) |
|---|---|---|---|---|
| Worst grade 1, n (%) | 37 | (37) | 13 | (32) |
| Worst grade 2, n (%) | 45 | (45) | 24 | (59) |
| Worst grade 3, n (%) | 8 | (8) | 1 | (2) |
| Worst grade 4, n (%) | 3 | (3) | 0 | |
| Worst grade 5, n (%) | 1 | (1) | 0 | |
| Median (range) time to | 2.0 | (1.0-12.0) | 2.0 | (1.0-8.0) |
| onset, days | | | | |
| Median (range) duration, days | 7.0 | (2.0-58.0) | 6.5 | (2.0-16.0) |

Neurologic events

| Any, n (%) | 65 | (64) | 25 | (61) |
|---|---|---|---|---|
| Worst grade 1, n (%) | 21 | (21) | 14 | (34) |
| Worst grade 2, n (%) | 15 | (15) | 4 | (10) |
| Worst grade 3, n (%) | 27 | (27) | 7 | (17) |
| Worst grade 4, n (%) | 2 | (2) | 0 | |
| Worst grade 5, n (%) | 0 | | 0 | |
| Median (range) time to | 5.0 | (1.0-17.0) | 6.0 | (1.0-93.0) |
| onset, days | | | | |
| Median (range) duration, days | 12.0 | (1.0-450.0) | 8.0 | (1.0-144.0) |

CRS, cytokine release syndrome;
TEAE, treatment-emergent adverse event

### Example 2

In the Zuma-1 pivotal study, grade 3+(G3+) cytokine release syndrome (CRS) and neurological events (NE) were observed in 13% and 28% of patients with median onset of any grade at 2 and 5 days, respectively (Neelapu S S et al., N Engl J Med. 2017; 377(26):2531-2544). Using conventional low throughput platforms such as multiplex ELISA, pretreatment markers, including serum levels of LDH, IL-6, and IL-15, demonstrated positive association with G3+ CRS and/or NE (Locke F L et al., Blood Advances. 2020; 4(19):4898-4911). We sought to explore biological mechanisms and novel markers underlying the development of early onset (within 5 days post-CAR T infusion) G3+ CRS and NE by high throughput Olink proteomic profiling.

Serum samples collected prior to conditioning chemotherapy (baseline) and immediately prior to CAR T therapy (day 0) for 142 R/R LBCL patients treated in Zuma-1 Phase 2. Table 10.

TABLE 10
Patient population and analysis set.

| | Overall | Cohort 1&2 | Cohort 4 |
|---|---|---|---|
| Binary Outcomes | (n = 142) | (n = 101) | (n = 41) |
| Early NE/CRS Tox (G3+ | 8 (6%) | 8 (8%) | 0 (0%) |
| w/in 3 days) | | | |
| G3+ CRS Tox (w/in 3 days) | 3 (2%) | 3 (3%) | 0 (0%) |
| G3+ NE Tox (w/in 3 days) | 5 (4%) | 5 (5%) | 0 (0%) |
| G3+ CRS Tox (w/in 5 days) | 8 (6%) | 7 (7%) | 1 (2%) |
| G3+ NE Tox (w/in 5 days) | 17 (12%) | 14 (14%) | 3 (7%) |
| G3+ CRS Tox (w/in 7 days) | 10 (7%) | 9 (9%) | 1 (2%) |
| G3+ NE Tox (w/in 5 days) | 30 (21%) | 25 (25%) | 5 (12%) |

Baseline serum samples were collected before conditioning (Day −5) or at leukapheresis. Cohorts 1, 2 and 4 were analyzed by Olink panels comprising 1,458 protein markers. Olink proteomic panels use a proprietary Proximity Extension Assay (PEA) technology, a unique method where each biomarker is detected by a matched pair of antibodies, coupled to unique, partially complementary oligonucleotides and measured by quantitative PCR. Olink. (2020). PEA—a high-multiplex immunoassay technology with qPCR or NGS readout. Olink. www.olink.com/application/pea-a-high-multiplex-immunoassay-technology-with-qpcr-or-ngs-readout-2/.

Association between marker expression and early G3+ toxicity was evaluated using Wilcoxon test and logistic regression. WGCNA (Weighted gene coexpression network analysis; Langfelder P. et al., BMC Bioinformatics, 2008; 9:559) was performed to identify highly co-expressed protein clusters, which were used for gene ontology (GO) analysis for biological interpretation. Machine learning methods were used to select features and build classifier with performance evaluated using mean AUC in the test set.

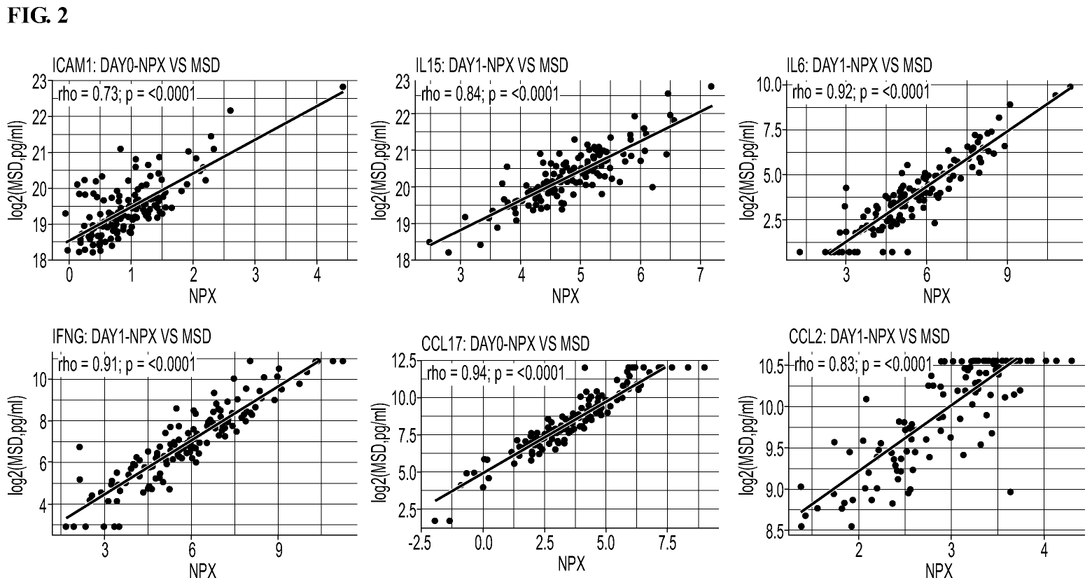

Olink analysis demonstrated a high degree of correlation and wider detection range compared the same markers (selected) analyzed by MSD (MesoScale Discovery, multiplex ELISA). FIG. 2. Olink often has higher sensitivity than MSD at both lower and higher expression ranges. NPX, Normalized Protein eXpression, is Olink's arbitrary unit which is in Log2 scale. It is calculated from Ct values and data pre-processing (normalization) is performed to minimize both intra- and inter-assay variation. NPX data allows users to identify changes for individual protein levels across their sample set, and then use this data to establish protein signatures. NPX values for 2 different analytes/proteins are not directly comparable.

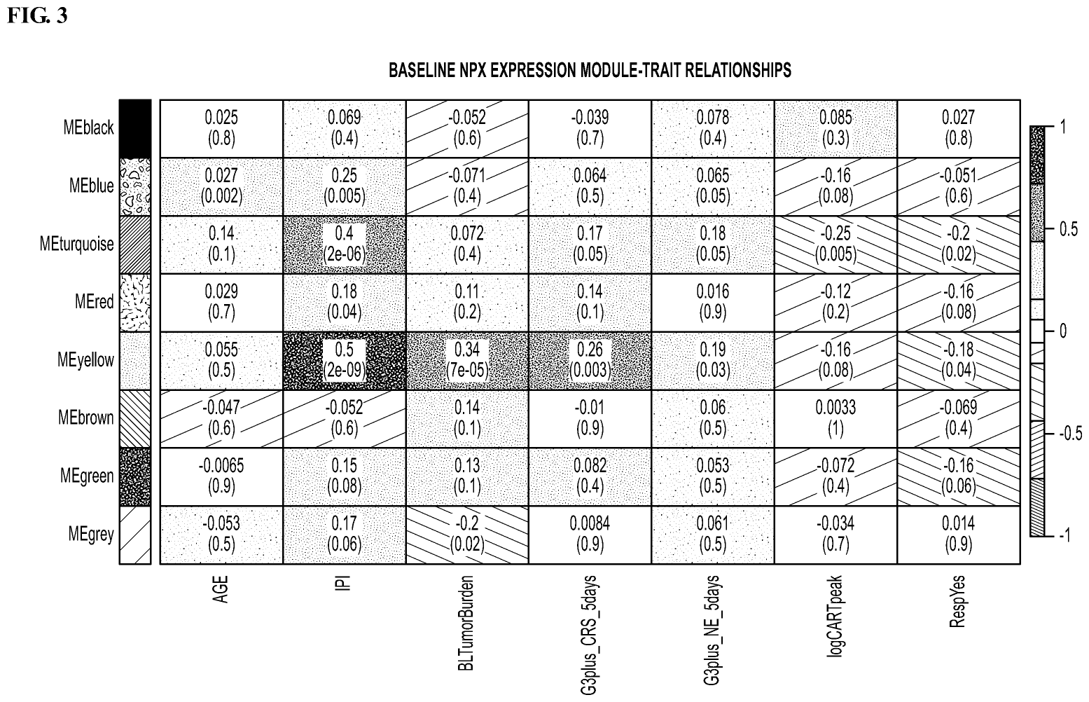

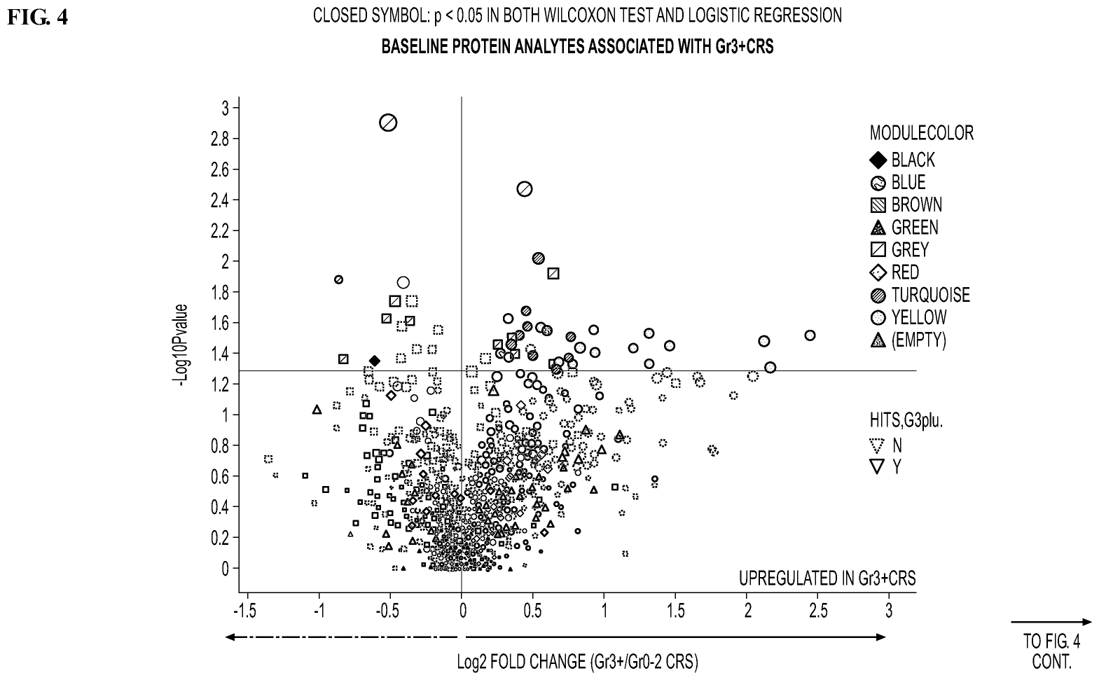

Twenty-four patients (17%) experienced G3+ CRS and/or NE within 5 days following CAR T infusion. Univariate and WGCNA analyses demonstrated that clusters of pretreatment markers associated with these toxicities, and they correlated positively with poor prognosis factors, e.g., international prognostic index and baseline tumor burden. WGCNA analysis showed baseline (collected at leukapheresis or prior to chemo-conditioning at Day −5) proteins co-expressed in “turquoise” and “yellow” modules positively associated with early toxicity (Grade 3+ NE and/or CRS toxicity) but showed inverse correlation with response. FIG. 3. A greater association was observed with the “yellow” module, which also associates with SPD and IPI score. For early high grade toxicity, correlation of the “yellow” module eigenprotein is stronger with CRS than NE (correlation coefficient r=0.26 vs. r=0.19, respectively). Meanwhile, the eigenprotein of the “turquoise” module showed positive correlation with IPI score while the eigenprotein of the “yellow” module positively correlated with both IPI and baseline tumor burden (SPD). The genes associated with the “yellow” and “turquoise” models at the baseline timepoint can be found in Table 14. The module color is based on the likelihood of coexpression.

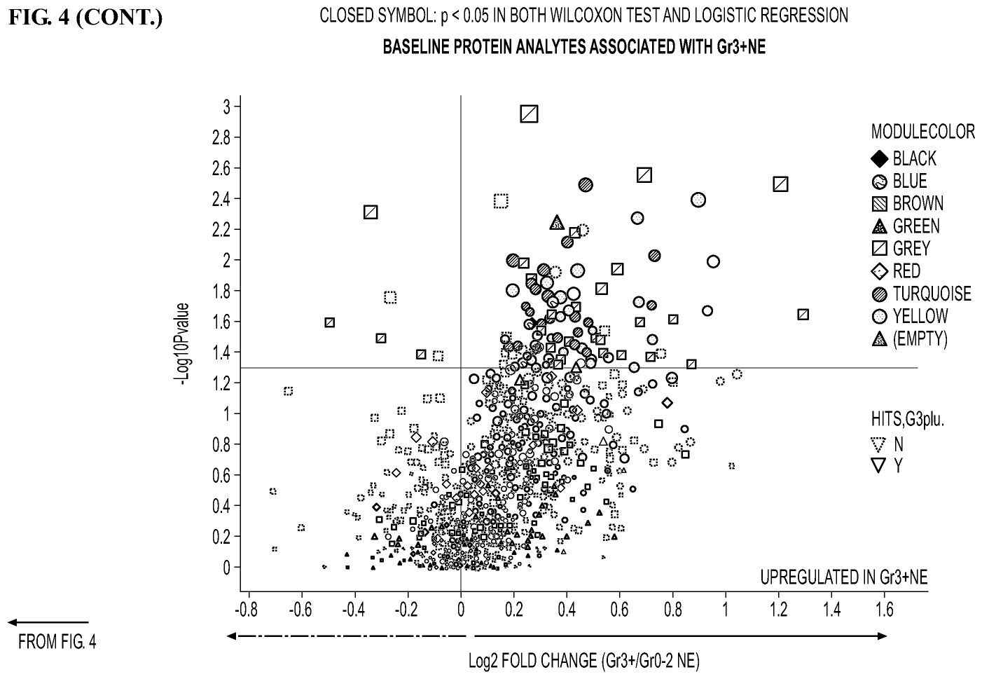

Volcano graphs of univariate analysis comparing Gr3+ with Gr0-2 CRS (left) or NE (right) were plotted. FIG. 4. Color labels are based on WGCNA color modules. Volcano plots depict differential expression of analytes selected through multivariable feature selection methods; x-axis reflects the median difference in NPX between those patients with Grade 3+ CRS/NE versus those without; y-axis reflects p-value from univariate analysis (Wilcoxon rank sum test), which is then transformed as −log₁₀(pvalue). Analyte color reflects its WGCNA module membership. A closed symbol represents protein analyte showing statistical significance (p<0.05) in both Wilcoxon test and Logistic Regression.

Hypergeometric test was used to identify the overall enrichment of the modules containing proteins associated with Gr3+ NE and/or CRS. Confirmation of modules of interest by cross comparing univariate and WGCNA analysis using hypergeometric test was observed. The analysis confirmed “turquoise” and “yellow” modules showed greatest association with early toxicity. The “turquoise” module positively associated with IPI and negatively with response. The “yellow” module positively associated with IPI and SPD, and negatively associated with response.

TABLE 11
Identification of protein sets (modules) at baseline associated with Gr3+ CRS or NE

| | | | | | | | |
|---|---|---|---|---|---|---|---|
| moduleColors | Freq | moduleSize | DEinModule | DEinbackground | backgroundSize | ratio | hyperPvalue |

Gr3+ CRS

| | | | | | | | |
|---|---|---|---|---|---|---|---|
| black | 35 | 35 | 0 | 207 | 1457 | 0 | 1.00E+00 |
| blue | 178 | 178 | 6 | 207 | 1457 | 0.03370787 | 1.00E+00 |
| brown | 125 | 125 | 2 | 207 | 1457 | 0.016 | 1.00E+00 |
| green | 84 | 84 | 8 | 207 | 1457 | 0.0952381 | 9.30E−01 |
| grey | 629 | 629 | 48 | 207 | 1457 | 0.07631161 | 1.00E+00 |
| red | 57 | 57 | 8 | 207 | 1457 | 0.14035088 | 5.75E−01 |
| Turquoise | 224 | 224 | 56 | 207 | 1457 | 0.25 | 1.83E−06 |
| yellow | 125 | 125 | 79 | 207 | 1457 | 0.632 | 1.34E−41 |

Gr3+ NE

| | | | | | | | |
|---|---|---|---|---|---|---|---|
| black | 35 | 35 | 1 | 91 | 1457 | 0.02857143 | 0.898216151 |
| blue | 178 | 178 | 6 | 91 | 1457 | 0.03370787 | 0.976127383 |
| brown | 125 | 125 | 1 | 91 | 1457 | 0.008 | 0.999783581 |
| green | 84 | 84 | 0 | 91 | 1457 | 0 | 1 |
| grey | 629 | 629 | 33 | 91 | 1457 | 0.05246423 | 0.931888502 |
| red | 57 | 57 | 1 | 91 | 1457 | 0.01754386 | 0.976507425 |
| turquoise | 224 | 224 | 30 | 91 | 1457 | 0.13392857 | 1.14178E−05 |
| yellow | 125 | 125 | 19 | 91 | 1457 | 0.152 | 0.000129793 |

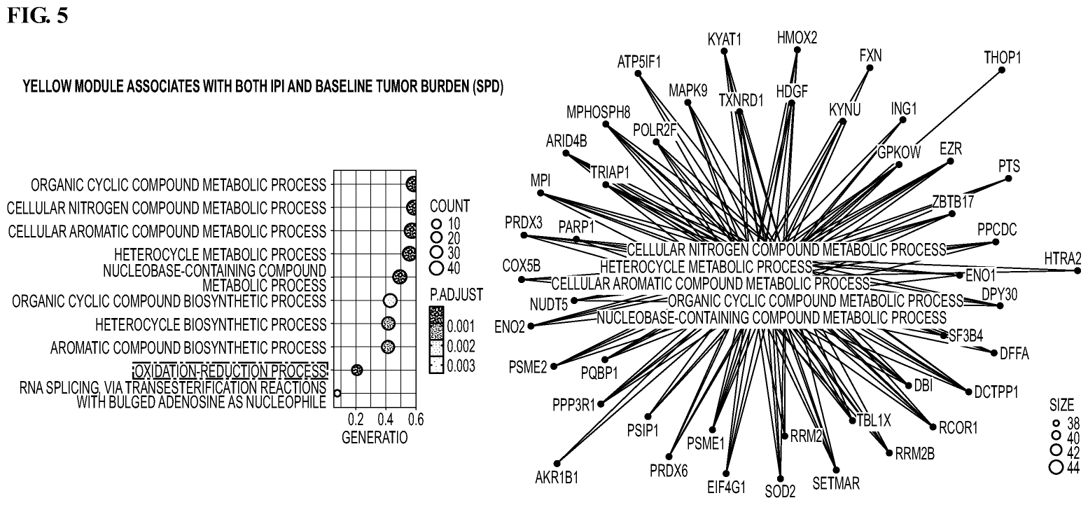

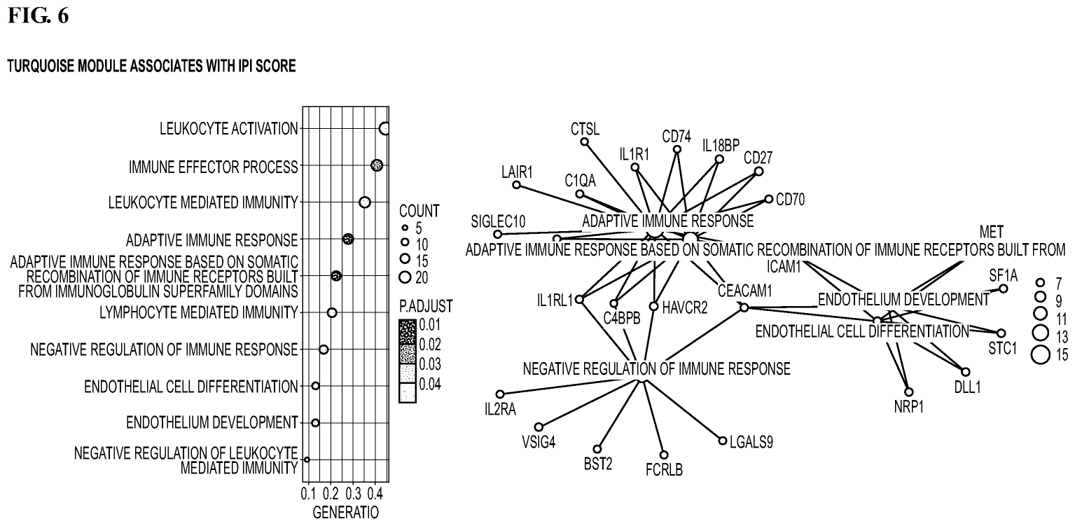

Gene ontology (GO) analysis applied to these clusters showed enrichment of proteins involved in metabolic processes and leukocyte activation. Differentially expressed proteins in the “yellow” module associated with response to oxidative stress and metabolic processes. FIG. 5. Oxidative stress may be correlated with hypoxia of tumors. Again, the “yellow” module associated with both IPI and baseline tumor burden (SPD). On the other hand, differentially expressed baseline proteins in the “turquoise” module positively associated with leukocyte activation and adhesion. FIG. 6. “Turquoise” module associated with IPI score. Systemic inflammation is associated with prognostic factor IPI score.

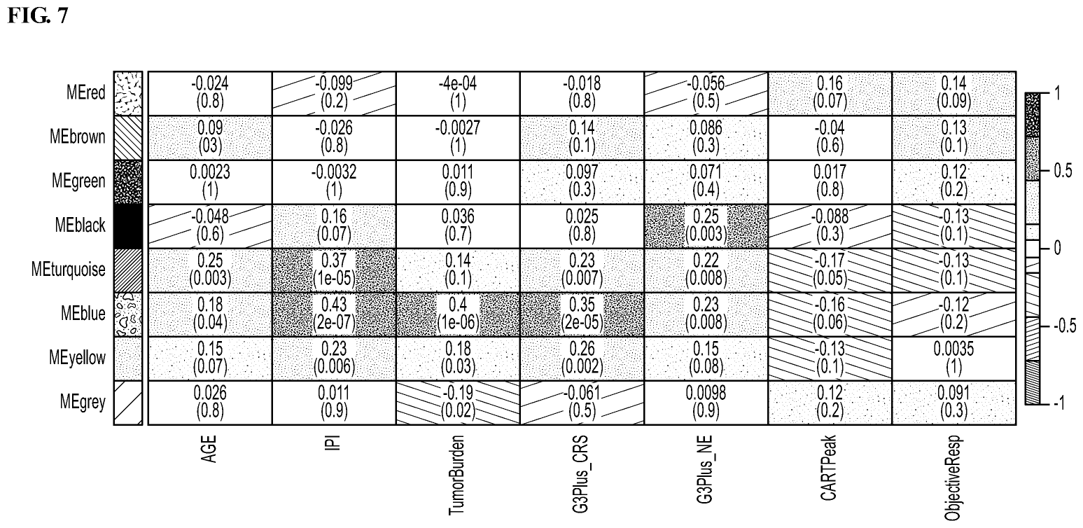

The O-Link WGCNA analysis was repeated at Day zero. FIG. 7. WGCNA analysis showed Day 0 (collected prior to CAR-T infusion) proteins co-expressed in “turquoise”, “blue”, “yellow”, and “black” modules positively associated with early toxicity. Meanwhile, the “turquoise” module showed positive correlation with IPI scores while the “blue” and “yellow” modules positively correlated with both IPI and baseline tumor burden (SPD). The “black” module, however, demonstrated no correlation with either baseline IPI or SPD. “Blue” and “Turquoise” modules show positive association with both Gr3+ CRS and NE. “Yellow” and “Black” modules preferentially associate with CRS and NE, respectively.

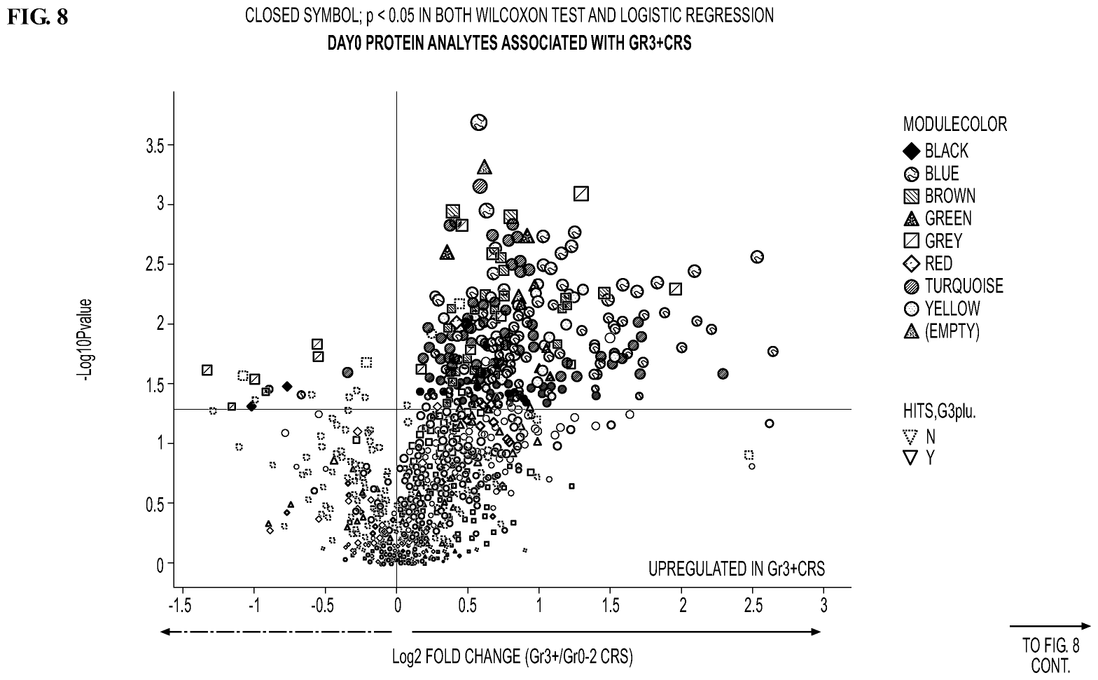

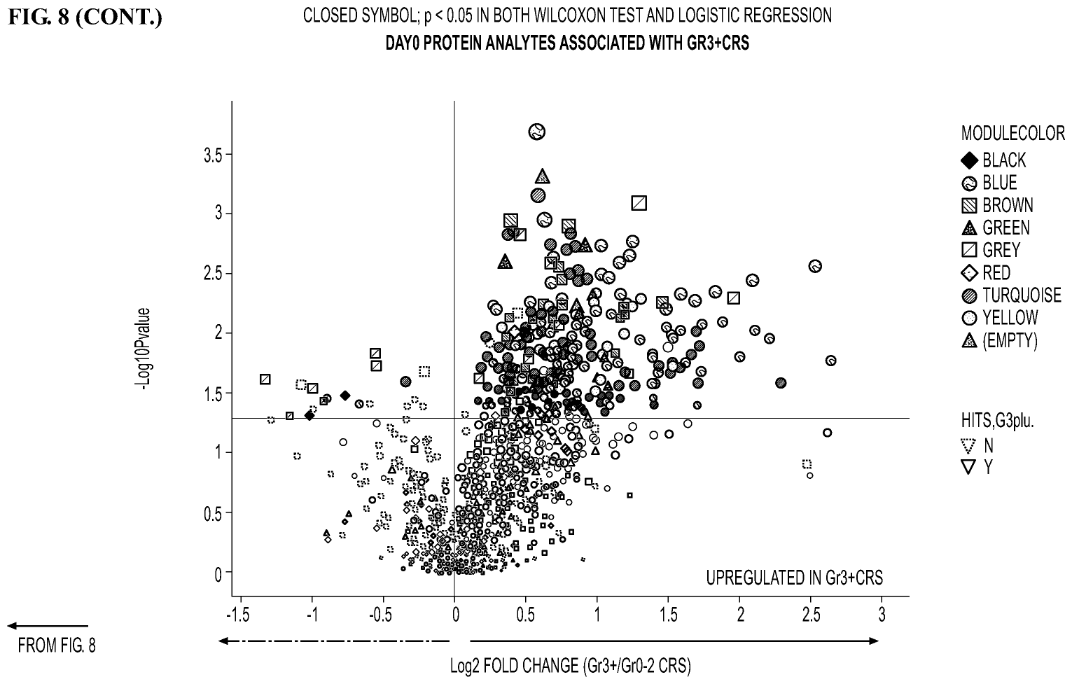

Volcano graphs of univariate analysis comparing Gr3+ with Gr0-2 CRS (FIG. 8 left) or NE (FIG. 8, right) were also plotted. Color label is based on WGCNA color modules. Volcano plots depict differential expression of analytes selected through multivariable feature selection methods; x-axis reflects the median difference in NPX between those patients with Grade 3+ CRS/NE versus those without; y-axis reflects p-value from univariate analysis (Wilcoxon rank sum test), which is then transformed as −log₁₀(pvalue). Analyte color reflects its WGCNA module membership. A closed symbol represents protein analyte showing statistical significance (p<0.05) in both Wilcoxon test and Logistic Regression. Hypergeometric test was used to identify the overall enrichment of the modules containing proteins associated with Gr3+early toxicity. The analysis confirmed “turquoise”, “blue”, and “yellow” modules as showing greatest association with early toxicity. In addition, the “blue” module associated positively with IPI and SPD, the “turquoise” module associated positively with IPI, and the “yellow” module associated positively with IPI and SPD.

TABLE 12
Identification of protein sets (modules) at Day 0 associated with Gr3+ CRS or NE: hypergeometric test.

| | | | | | | | |
|---|---|---|---|---|---|---|---|
| Module | | | | | | | |
| Colors | Freq | moduleSize | DEinModule | DEinbackground | backgroundSize | ratio | hyperPvalue |

Gr3+ CRS

| | | | | | | | |
|---|---|---|---|---|---|---|---|
| black | 37 | 37 | 1 | 361 | 1457 | 0.02702703 | 1.00E+00 |
| blue | 184 | 184 | 99 | 361 | 1457 | 0.53804348 | 7.47E−20 |
| brown | 169 | 169 | 32 | 361 | 1457 | 0.18934911 | 9.78E−01 |
| green | 81 | 81 | 16 | 361 | 1457 | 0.19753086 | 8.89E−01 |
| grey | 364 | 364 | 29 | 361 | 1457 | 0.07967033 | 1.00E+00 |
| red | 45 | 45 | 2 | 361 | 1457 | 0.04444444 | 1.00E+00 |
| turquoise | 494 | 494 | 140 | 361 | 1457 | 0.28340081 | 1.46E−02 |
| yellow | 83 | 83 | 42 | 361 | 1457 | 0.5060241 | 1.50E−07 |

Gr3+ NE

| | | | | | | | |
|---|---|---|---|---|---|---|---|
| black | 37 | 37 | 9 | 241 | 1457 | 0.24324324 | 1.44E−01 |
| blue | 184 | 184 | 58 | 241 | 1457 | 0.31521739 | 4.49E−08 |
| brown | 169 | 169 | 14 | 241 | 1457 | 0.08284024 | 1.00E+00 |
| green | 81 | 81 | 3 | 241 | 1457 | 0.03703704 | 1.00E+00 |
| grey | 364 | 364 | 26 | 241 | 1457 | 0.07142857 | 1.00E+00 |
| red | 45 | 45 | 0 | 241 | 1457 | 0 | 1.00E+00 |
| turquoise | 494 | 494 | 113 | 241 | 1457 | 0.22874494 | 3.30E−06 |
| yellow | 83 | 83 | 18 | 241 | 1457 | 0.21686747 | 1.27E−01 |

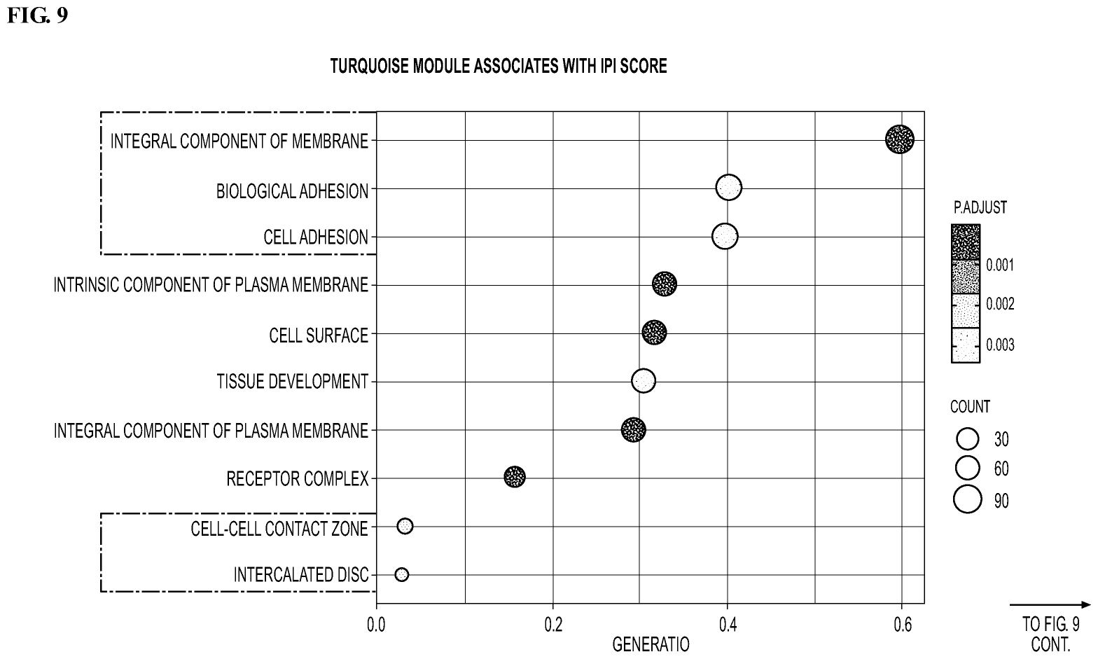

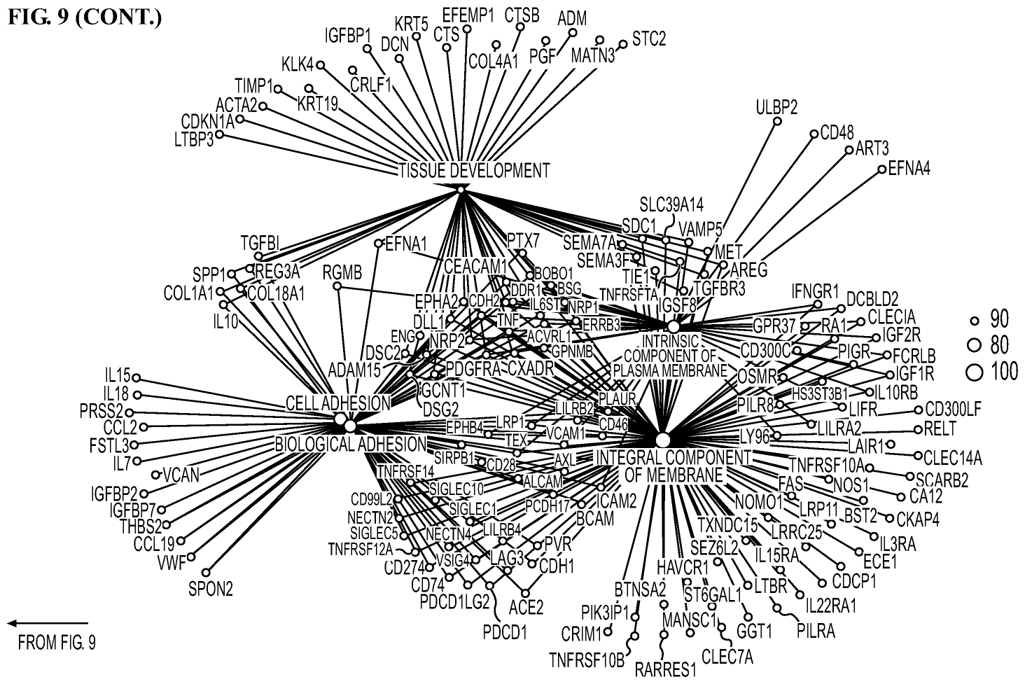

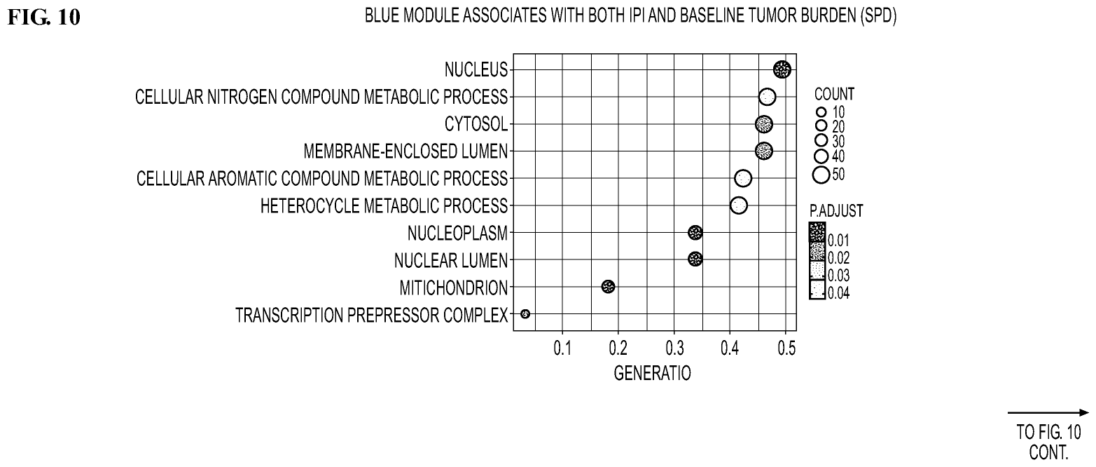

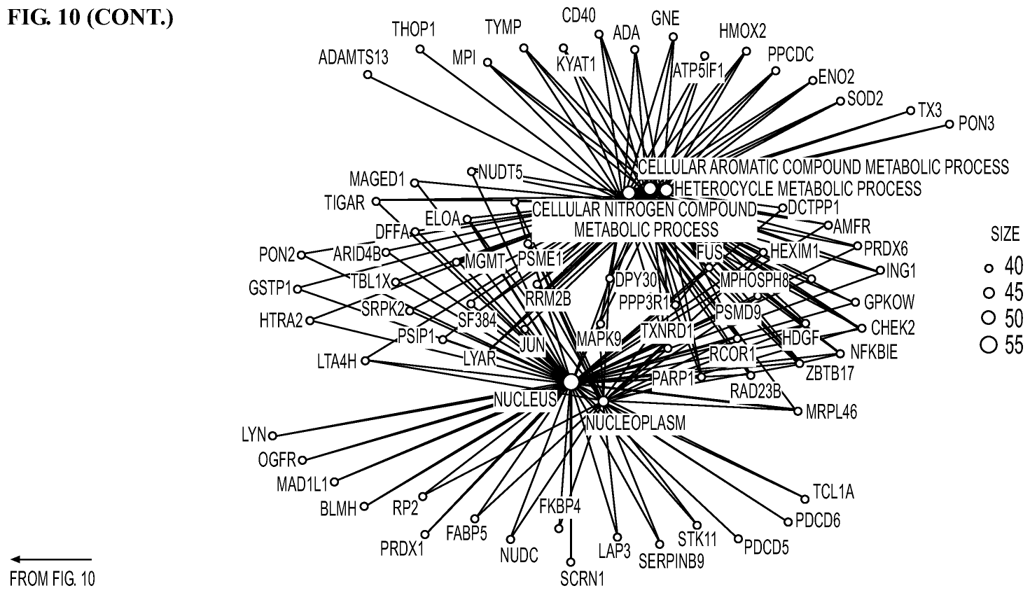

GO analysis at Day 0 showed that Day 0 proteins related to leukocyte activation and trafficking are enriched in “turquoise” module. FIG. 9. “Turquoise” module associates with IPI score and cell adhesion. Similar GO was observed with baseline analytes. Differentially expressed proteins in “blue” module associates with metabolic process. FIG. 10. “Yellow” module shows similar pathways with addition of autophagy pathway. Similar GO was observed with baseline analytes.

Notably, there was a higher preponderance of day 0 analytes as compared to baseline in marker clusters associated with toxicities (CRS: 292 vs. 133 markers; NE: 181 vs. 63 markers). In addition, eosinophil and monocyte counts also negatively associated with toxicity. It is possibly due to biological relevance and contribution of lymphodepleting conditioning amplifying immune activation and vasculature damage.

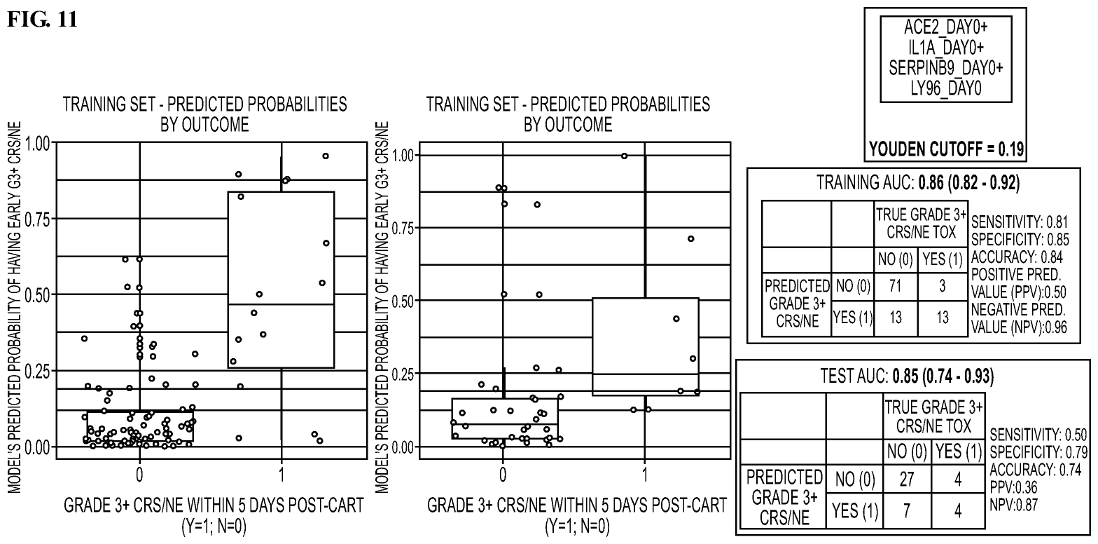

Machine learning methods demonstrated excellent performance (mean AUC>0.80 in both training and testing sets of groups of Olink markers in classifying patients with early high-grade toxicity. Table 13. Pre-CAR-T cytokines 0-Link analytes have good classification performance for Grade 3+ CRS/NE toxicity within 5 days post-CAR-T. ACE2_DAY0, IL1A_DAY0, SERPINB9_DAY0, and LY96_DAY0 had test AUC 0.85 (0.74-0.93). FIG. 11.

TABLE 13

| | | Train AUC | Test AUC |
|---|---|---|---|
| No. | Variable Set | (95% CI) | (95% CI) |
| 1 | ACE2 (day 0); IL1A (day 0); SERPINB9 (day 0); LY96 (day | 0.86 (0.83- | 0.85 (0.74- |
| | 0); | 0.92) | 0.93) |
| 2 | ACE2 (day 0); IL1A (day 0); SERPINB9 (day 0); LY96 (day | 0.86 (0.82- | 0.84 (0.74- |
| | 0); REG1A (day 0) | 0.92) | 0.93) |
| 3 | OSMR (day 0); REG1B (day 0); LY96 (day 0); ACE2 (day | 0.87 (0.83- | 0.84 (0.70- |
| | 0); LY9 (day 0); IL1A (day 0); SERPINB9 (day 0) | 0.92) | 0.93) |
| 4 | REG3A (day 0); REG1B (day 0); LY96 (day 0); ACE2 (day | 0.89 (0.85- | 0.84 (0.73- |
| | 0); LY9 (day 0); IL1A (day 0); SERPINB9 (day 0) | 0.92) | 0.92) |
| 5 | KYNU (baseline); LY96 (day 0); MTPN (day 0); ACAA1 | 0.88 (0.84- | 0.84 (0.70- |
| | (baseline); MCP1 (day 0); LDH (baseline) | 0.92) | 0.95) |
| 6 | SERPINB9 (day 0); REG1B (day 0); SOD2 (day 0); ACE2 | 0.85 (0.82- | 0.83 (0.71- |
| | (day 0); LY9 (day 0); IL1A (day 0) | 0.90) | 0.92) |
| 7 | REG1B (day 0); ACE2 (day 0); LY9 (day 0); IL1A (day 0) | 0.84 (0.80- | 0.83 (0.72- |
| | | 0.89) | 0.92) |
| 8 | OSMR (day 0); REG1B (day 0); LY96 (day 0); ACE2 (day | 0.86 (0.82- | 0.83 (0.72- |
| | 0); LY9 (day 0); IL1A (day 0) | 0.91) | 0.93) |
| 9 | KYNU (baseline); LY96 (day 0); REG3A (day 0); | 0.91 (0.88- | 0.82 (0.72- |
| | CEACAM1 (day 0); ACE2 (day 0); VAMP5 (day 0); MTPN | 0.95) | 0.92) |
| | (day 0); ACAA1 (baseline); MCP1 (day 0); LDH (baseline) | | |
| 10 | KYNU (baseline); LY96 (day 0); REG3A (day 0); | 0.88 (0.83- | 0.81 (0.71- |
| | CEACAM1 (day 0); ACE2 (day 0); VAMP5 (day 0); MTPN | 0.93) | 0.93) |
| | (day 0); ACAA1 (baseline) | | |

In addition, we observed that IL1/IL6 pathway markers, including IL1A and OSMR, are useful in identifying G3+ CRS, while inflammatory endothelial markers, such as ACE2, CEACAM1, ICAM2, and ADAM15, could classify patients with both G3+ CRS and NE upon Axi-cel treatment. Pre-CART cytokines 0-Link analytes have good prediction performance for Grade 3+ CRS/NE toxicity within 5 days post-CAR-T. In FIG. 11 and Table 13, multivariable feature selection methods and machine learning modeling were employed to identify important analytes and develop well-performing models for classifying Grade 3+ CRS/NE. Feature selection methods, including penalized logistic regression and random forest, were used to identify top analytes important in explaining Grade 3+ CRS/NE; a logistic classifier model was built using some of the top features selected; performance metrics for 70% training and 30% test set are summarized for one of the top-performing classification models; mean AUC with 90% empirical confidence interval are presented.

Based on machine learning models, the following markers demonstrate good performance. By cross comparing with univariate analysis, LPL (baseline) is downregulated in patients with Gr3+ CRS, while MET (baseline), NELL2 (baseline), OSMR (baseline), REG3A (baseline), AREG (day 0), CKAP4 (day 0), CXCL1 (day 0), EIF5A (day 0), IL1A (day 0), KIFBP (day 0), KIRREL2 (day 0), NUB1 (day 0), PAG1 (day 0), PCDH17 (day 0), STK11 (day 0), TGFB1 (day 0) are upregulated in patients with Gr3+ CRS. The following are associated with Gr3+ NE: CCL16 (day 0), CELA3A (day 0), MEGF9 (day 0), MFAP3 (day 0), and REG1B (day 0). The following are upregulated in patients with both, Gr3+ CRS and Gr3+ NE: baseline KYNU (baseline), ACE2 (day 0), ADAM15 (day 0), BSG (day 0), CEACAM1 (day 0), HLA-DRA (day 0), ICAM2 (day 0), LY9 (day 0), LY96 (day 0), OSMR (day 0), REG3A (day 0), SERPINB9 (day 0), SOD2 (day 0), and VAMP5 (day 0). Furthermore, we observed that IL1/IL6 pathway associated markers, including IL1A and OSMR, are potentially useful in identifying G3+ CRS, while inflammatory endothelial markers, such as ACE2, CEACAM1, ICAM2, and ADAM15, could classify patients with both G3+ CRS and NE upon Axi-cel treatment. Ferritin, AST and TNFa positively correlate with Olink biomarkers identified by machine learning.

In general, we observed that IL-1/IL-6 pathway markers (eg, IL-1A and OSMR) are useful in identifying Grade ≥3 CRS, while inflammatory endothelial markers (eg, ACE2, CEACAM1, ICAM2, and ADAM15) could classify patients with both Grade ≥3 CRS and NE upon axi-cel treatment.

TABLE 14
Module color protein analysis at baseline

| Assay | moduleColor | Gene | Visit |
|---|---|---|---|
| ANXA5_BASELINE | turquoise | ANXA5 | BASELINE |
| AREG_BASELINE | turquoise | AREG | BASELINE |
| AXL_BASELINE | turquoise | AXL | BASELINE |
| B4GALT1_BASELINE | turquoise | B4GALT1 | BASELINE |
| BLMH_BASELINE | yellow | BLMH | BASELINE |
| COX5B_BASELINE | yellow | COX5B | BASELINE |
| DPY30_BASELINE | yellow | DPY30 | BASELINE |
| GFER_BASELINE | yellow | GFER | BASELINE |
| IL1RL1_BASELINE | turquoise | IL1RL1 | BASELINE |
| ITIH3_BASELINE | turquoise | ITIH3 | BASELINE |
| MAPK9_BASELINE | yellow | MAPK9 | BASELINE |
| MET_BASELINE | turquoise | MET | BASELINE |
| MPHOSPH8_BASELINE | yellow | MPHOSPH8 | BASELINE |
| OSMR_BASELINE | turquoise | OSMR | BASELINE |
| PRDX3_BASELINE | yellow | PRDX3 | BASELINE |
| PREB_BASELINE | yellow | PREB | BASELINE |
| RRM2_BASELINE | yellow | RRM2 | BASELINE |
| SOD2_BASELINE | yellow | SOD2 | BASELINE |
| THOP1_BASELINE | yellow | THOP1 | BASELINE |
| TXNDC15_BASELINE | turquoise | TXNDC15 | BASELINE |
| ZBTB17_BASELINE | yellow | ZBTB17 | BASELINE |
| ADA_BASELINE | yellow | ADA | BASELINE |
| AMFR_BASELINE | yellow | AMFR | BASELINE |
| BCAM_BASELINE | turquoise | BCAM | BASELINE |
| BST2_BASELINE | turquoise | BST2 | BASELINE |
| CASC4_BASELINE | turquoise | CASC4 | BASELINE |
| CLTA_BASELINE | yellow | CLTA | BASELINE |
| CSF1_BASELINE | turquoise | CSF1 | BASELINE |
| CSTB_BASELINE | turquoise | CSTB | BASELINE |
| CTSL_BASELINE | turquoise | CTSL | BASELINE |
| CXCL11_BASELINE | turquoise | CXCL11 | BASELINE |
| DBI_BASELINE | yellow | DBI | BASELINE |
| DECR1_BASELINE | yellow | DECR1 | BASELINE |
| FXYD5_BASELINE | turquoise | FXYD5 | BASELINE |
| GNLY_BASELINE | turquoise | GNLY | BASELINE |
| ITGA5_BASELINE | turquoise | ITGA5 | BASELINE |
| KYNU_BASELINE | yellow | KYNU | BASELINE |
| LGALS9_BASELINE | turquoise | LGALS9 | BASELINE |
| LILRB2_BASELINE | turquoise | LILRB2 | BASELINE |
| LILRB4_BASELINE | turquoise | LILRB4 | BASELINE |
| LRMP_BASELINE | yellow | LRMP | BASELINE |
| LTBP2_BASELINE | turquoise | LTBP2 | BASELINE |
| LY9_BASELINE | turquoise | LY9 | BASELINE |
| LYN_BASELINE | yellow | LYN | BASELINE |
| MGMT_BASELINE | yellow | MGMT | BASELINE |
| MPI_BASELINE | yellow | MPI | BASELINE |
| NFKBIE_BASELINE | yellow | NFKBIE | BASELINE |
| NRP2_BASELINE | turquoise | NRP2 | BASELINE |
| PARP1_BASELINE | yellow | PARP1 | BASELINE |
| PDCD6_BASELINE | yellow | PDCD6 | BASELINE |
| PPCDC_BASELINE | yellow | PPCDC | BASELINE |
| PTS_BASELINE | yellow | PTS | BASELINE |
| PVR_BASELINE | turquoise | PVR | BASELINE |
| RBP5_BASELINE | turquoise | RBP5 | BASELINE |
| SPINK1_BASELINE | turquoise | SPINK1 | BASELINE |
| TIMP1_BASELINE | turquoise | TIMP1 | BASELINE |
| TINAGL1_BASELINE | turquoise | TINAGL1 | BASELINE |
| TRIAP1_BASELINE | yellow | TRIAP1 | BASELINE |
| TXNRD1_BASELINE | yellow | TXNRD1 | BASELINE |
| VCAN_BASELINE | turquoise | VCAN | BASELINE |
| VWF_BASELINE | turquoise | VWF | BASELINE |

TABLE 15
Module color protein analysis at Day 0

| Assay | ProteinNames | GeneNames | moduleColor |
|---|---|---|---|
| AARSD1 | Alanyl-tRNA editing protein Aarsd1 | AARSD1 | yellow |
| ABHD14B | Abhydrolase Domain Containing 14B | ABHD14B CIB | yellow |
| ABL1 | Tyrosine-protein kinase ABL1 | ABL1 ABL JTK7 | yellow |
| ADAM8 | Disintegrin and metalloproteinase domain- | ADAM8 MS2 | yellow |
| | containin | | |
| AHCY | Adenosylhomocysteinase (AdoHcyase) | AHCY SAHH | yellow |
| AHSP | Alpha-hemoglobin-stabilizing protein | AHSP EDRF ERAF | yellow |
| AK1 | Adenylate kinase isoenzyme 1 (AK 1) | AK1 | yellow |
| AKR1B1 | Aldo-keto reductase family 1 member B1 | AKR1B1 ALDR1 | yellow |
| | | ALR2 | |
| AKT1S1 | Proline-rich AKT1 substrate 1 | AKT1S1 PRAS40 | yellow |
| AKT3 | RAC-gamma serine/threonine-protein | AKT3 PKBG | yellow |
| | kinase | | |
| ANKRD54 | Ankyrin repeat domain-containing protein | ANKRD54 LIAR | yellow |
| | 54 | | |
| APRT | Adenine phosphoribosyltransferase (APRT) | APRT | yellow |
| ATG4A | autophagy related 4A cysteine peptidase | ATG4A APG4A | yellow |
| | | AUTL2 | |
| ATOX1 | Copper transport protein ATOX1 | ATOX1 HAH1 | yellow |
| BAG6 | BAG cochaperone 6 | BAG6 BAT3 G3 | yellow |
| BLVRB | biliverdin reductase B | BLVRB FLR | yellow |
| CA1 | Carbonic anhydrase 1 | CA1 | yellow |
| CA2 | Carbonic anhydrase 2 | CA2 | yellow |
| CARHSP1 | Calcium-regulated heat-stable protein 1 | CARHSP1 | yellow |
| CASP8 | Caspase-8 | CASP8 MCH5 | yellow |
| CCS | Copper chaperone for superoxide dismutase | CCS | yellow |
| CCT5 | T-complex protein 1 subunit epsilon | CCT5 CCTE | yellow |
| | | KIAA0098 | |
| CD2AP | CD2-associated protein (Adapter protein | CD2AP | yellow |
| | CMS) | | |
| CDC37 | Hsp90 co-chaperone Cdc37 | CDC37 CDC37A | yellow |
| CEP164 | Centrosomal protein of 164 kDa (Cep164) | CEP164 KIAA1052 | yellow |
| | | NPHP15 | |
| CNPY2 | Protein canopy homolog 2 | CNPY2 MSAP | yellow |
| | | TMEM4 ZSIG9 | |
| | | UNQ1943/PRO4426 | |
| CPPED1 | Serine/threonine-protein phosphatase | CPPED1 CSTP1 | yellow |
| | CPPED1 | | |
| DAPP1 | Dual adapter for phosphotyrosine and 3- | DAPP1 BAM32 | yellow |
| | phosphotyro | HSPC066 | |
| DBI | Acyl-CoA-binding protein (ACBP) | DBI | yellow |
| DCTN2 | Dynactin subunit 2 | DCTN2 DCTN50 | yellow |
| DCTN6 | Dynactin subunit 6 (Dynactin subunit p27) | DCTN6 WS3 | yellow |
| DNAJB1 | DnaJ homolog subfamily B member 1 | DNAJB1 DNAJ1 | yellow |
| | | HDJ1 HSPF1 | |
| EBAG9 | estrogen receptor binding site associated | EBAG9 RCAS1 | yellow |
| | antigen 9 | | |
| EIF4B | Eukaryotic translation initiation factor 4B | EIF4B | yellow |
| EIF4G1 | Eukaryotic translation initiation factor 4 | EIF4G1 EIF4F | yellow |
| | gamma 1 | EIF4G EIF4GI | |
| ENO1 | Alpha-enolase | ENO1 ENO1L1 | yellow |
| | | MBPB1 MPB1 | |
| FMR1 | Synaptic functional regulator FMR1 | FMR1 | yellow |
| FXYD5 | FXYD domain-containing ion transport | FXYD5 DYSAD | yellow |
| | regulator 5 | IWU1 HSPC113 | |
| | | UNQ2561/PRO6241 | |
| GLO1 | Lactoylglutathione lyase | GLO1 | yellow |
| GLRX | Glutaredoxin-1 (Thioltransferase-1) (TTase- | GLRX GRX | yellow |
| | 1) | | |
| GMPR | GMP reductase 1 (GMPR 1) | GMPR GMPR1 | yellow |
| HAGH | Hydroxyacylglutathione hydrolase, | HAGH GLO2 | yellow |
| | mitochondrial | HAGH1 | |
| HARS | Histidine--tRNA ligase, cytoplasmic | HARS1 HARS HRS | yellow |
| HBQ1 | Hemoglobin subunit theta-1 | HBQ1 | yellow |
| HGS | Hepatocyte growth factor-regulated tyrosine | HGS HRS | yellow |
| | kinase | | |
| HK2 | Hexokinase-2 | HK2 | yellow |
| HMBS | Porphobilinogen deaminase (PBG-D) | HMBS PBGD UPS | yellow |
| HPCAL1 | Hippocalcin-like protein 1 | HPCAL1 BDR1 | yellow |
| HSPA1A | Heat shock 70 kDa protein 1A | HSPA1A HSP72 | yellow |
| | | HSPA1 HSX70 | |
| IL1B | Interleukin-1 beta (IL-1 beta) (Catabolin) | IL1B IL1F2 | yellow |
| ILKAP | Integrin-linked kinase-associated | ILKAP | yellow |
| | serine/threonine | | |
| IMPA1 | Inositol monophosphatase 1 (IMP 1) | IMPA1 IMPA | yellow |
| | (IMPase 1) | | |
| LACTB2 | Lactamase beta 2 | LACTB2 CGI-83 | yellow |
| MAEA | E3 ubiquitin-protein transferase MAEA | MAEA EMP | yellow |
| | | HLC10 PIG5 | |
| MED18 | Mediator complex subunit 18 | MED18 | yellow |
| METAP2 | Methionine aminopeptidase 2 (MAP 2) | METAP2 MNPEP | yellow |
| | | P67EIF2 | |
| MIF | Macrophage migration inhibitory factor | MIF GLIF MMIF | yellow |
| | (MIF) | | |
| NUDT2 | Nudix hydrolase 2 | NUDT2 APAH1 | yellow |
| PARK7 | Parkinson disease protein 7 | PARK7 | yellow |
| PEBP1 | Phosphatidylethanolamine-binding protein 1 | PEBP1 PBP PEBP | yellow |
| PKLR | Pyruvate kinase PKLR | PKLR PK1 PKL | yellow |
| PLPBP | Pyridoxal phosphate homeostasis protein | PLPBP PROSC | yellow |
| PPME1 | Protein phosphatase methylesterase 1 | PPME1 PME1 | yellow |
| | (PME-1) | PP2593 PRO0750 | |
| PRKAR1A | protein kinase cAMP-dependent type I | PRKAR1A PKR1 | yellow |
| | regulatory subunit alpha | PRKAR1 TSE1 | |
| RILP | Rab-interacting lysosomal protein | RILP PP10141 | yellow |
| RNF41 | ring finger protein 41 | RNF41 FLRF | yellow |
| | | NRDP1 SBBI03 | |
| RWDD1 | RWD domain-containing protein 1 | RWDD1 DFRP2 | yellow |
| | | CGI-24 PTD013 | |
| S100A4 | Protein S100-A4 (Calvasculin) (Metastasin) | S100A4 CAPL | yellow |
| | | MTS1 | |
| SIRT2 | NAD-dependent protein deacetylase sirtuin- | SIRT2 SIR2L | yellow |
| | 2 | SIR2L2 | |
| STAMBP | STAM-binding protein | STAMBP AMSH | yellow |
| STIP1 | Stress-induced-phosphoprotein 1 (STI1) | STIP1 | yellow |
| STX6 | Syntaxin-6 | STX6 | yellow |
| TARBP2 | RISC-loading complex subunit TARBP2 | TARBP2 TRBP | yellow |
| TBCC | Tubulin-specific chaperone C | TBCC | yellow |
| TDRKH | Tudor and KH domain-containing protein | TDRKH TDRD2 | yellow |
| TGM2 | Protein-glutamine gamma- | TGM2 | yellow |
| | glutamyltransferase 2 | | |
| TPMT | Thiopurine S-methyltransferase | TPMT | yellow |
| TRAF2 | TNF receptor-associated factor 2 | TRAF2 TRAP3 | yellow |
| TRIM21 | E3 ubiquitin-protein ligase TRIM21 | TRIM21 RNF81 | yellow |
| | | RO52 SSA1 | |
| UBAC1 | Ubiquitin-associated domain-containing | UBAC1 GBDR1 | yellow |
| | protein 1 | KPC2 UBADC1 | |
| USO1 | General vesicular transport factor p115 | USO1 VDP | yellow |
| VAT1 | Synaptic vesicle membrane protein VAT-1 | VAT1 | yellow |
| | homolog | | |
| WWP2 | NEDD4-like E3 ubiquitin-protein ligase | WWP2 | yellow |
| | WWP2 | | |
| ACE2 | Angiotensin-converting enzyme 2 | ACE2 | turquoise |
| | | UNQ868/PRO1885 | |
| ACP5 | Tartrate-resistant acid phosphatase type 5 | ACP5 | turquoise |
| ACP6 | Lysophosphatidic acid phosphatase type 6 | ACP6 ACPL1 | turquoise |
| | | LPAP | |
| | | UNQ205/PRO231 | |
| ACTA2 | Actin, aortic smooth muscle (Alpha-actin-2) | ACTA2 ACTSA | turquoise |
| | | ACTVS GIG46 | |
| ACVRL1 | Serine/threonine-protein kinase receptor R3 | ACVRL1 | turquoise |
| | (SKR3) | ACVRLK1 ALK1 | |
| ADA2 | Adenosine deaminase 2 | ADA2 ADGF | turquoise |
| | | CECR1 IDGFL | |
| ADAM15 | ADAM metallopeptidase domain 15 | ADAM15 MDC15 | turquoise |
| ADAM22 | ADAM metallopeptidase domain 22 | ADAM22 MDC2 | turquoise |
| ADAM23 | ADAM metallopeptidase domain 23 | ADAM23 MDC3 | turquoise |
| ADAMTS15 | ADAM metallopeptidase with | ADAMTS15 | turquoise |
| | thrombospondin type 1 motif 15 | | |
| ADAMTS16 | ADAM metallopeptidase with | ADAMTS16 | turquoise |
| | thrombospondin type 1 motif 16 | KIAA2029 | |
| ADAMTS8 | ADAM metallopeptidase with | ADAMTS8 METH2 | turquoise |
| | thrombospondin type 1 motif 8 | | |
| ADGRB3 | Adhesion G protein-coupled receptor B3 | ADGRB3 BAI3 | turquoise |
| | | KIAA0550 | |
| ADGRE2 | Adhesion G protein-coupled receptor E2 | ADGRE2 EMR2 | turquoise |
| ADGRG1 | Adhesion G-protein coupled receptor G1 | ADGRG1 GPR56 | turquoise |
| | | TM7LN4 TM7XN1 | |
| | | UNQ540/PRO1083 | |
| ADM | Pro-adrenomedullin [Cleaved into: | ADM AM | turquoise |
| | Adrenomedullin] | | |
| AGER | Advanced glycosylation end product- | AGER RAGE | turquoise |
| | specific receptor | | |
| AGRN | Agrin [Cleaved into: Agrin N-terminal 110 | AGRN AGRIN | turquoise |
| | kDa subu | | |
| AGRP | Agouti-related protein | AGRP AGRT ART | turquoise |
| ALCAM | CD166 antigen (Activated leukocyte cell | ALCAM MEMD | turquoise |
| | adhesion molecule) | | |
| AMBP | Protein AMBP [Cleaved into: Alpha-1- | AMBP HCP ITIL | turquoise |
| | microglobulin precursor] | | |
| ANGPT2 | Angiopoietin-2 (ANG-2) | ANGPT2 | turquoise |
| ANGPTL1 | Angiopoietin-related protein 1 | ANGPTL1 ANG3 | turquoise |
| | (Angiopoietin-3) | ANGPT3 ARP1 | |
| | | PSEC0154 | |
| | | UNQ162/PRO188 | |
| ANGPTL2 | Angiopoietin-related protein 2 | ANGPTL2 ARP2 | turquoise |
| | | UNQ170/PRO196 | |
| ANGPTL4 | Angiopoietin-related protein 4 | ANGPTLA ARP4 | turquoise |
| | | HFARP PGAR | |
| | | PP1158 PSEC0166 | |
| | | UNQ171/PRO197 | |
| ANGPTL7 | Angiopoietin-related protein 7 | ANGPTL7 CDT6 | turquoise |
| | | UNQ313/PRO356 | |
| ANPEP | Aminopeptidase N (AP-N) (hAPN) | ANPEP APN CD13 | turquoise |
| | | PEPN | |
| APLP1 | Amyloid-like protein 1 (APLP) (APLP-1) | APLP1 | turquoise |
| APOH | Beta-2-glycoprotein 1 (APC inhibitor) | APOH B2G1 | turquoise |
| AREG | Amphiregulin (AR) | AREG AREGB | turquoise |
| | | SDGF | |
| ART3 | Ecto-ADP-ribosyltransferase 3 (EC | ART3 TMART | turquoise |
| | 2.4.2.31) | | |
| ASGR1 | Asialoglycoprotein receptor 1 (ASGP-R 1) | ASGR1 CLEC4H1 | turquoise |
| | (ASGPR 1) | | |
| AXL | Tyrosine-protein kinase receptor UFO | AXL UFO | turquoise |
| B4GALT1 | Beta-1,4-galactosyltransferase 1 | B4GALT1 GGTB2 | turquoise |
| BAG3 | BAG family molecular chaperone regulator | BAG3 BIS | turquoise |
| | 3 (BAG-3) | | |
| BAMBI | BMP and activin membrane-bound inhibitor | BAMBI NMA | turquoise |
| | homolog | | |
| BCAM | Basal cell adhesion molecule (Auberger B | BCAM LU MSK19 | turquoise |
| | antigen) | | |
| BCAN | Brevican core protein | BCAN BEHAB | turquoise |
| | | CSPG7 | |
| | | UNQ2525/PRO6018 | |
| BMP4 | Bone morphogenetic protein 4 (BMP-4) | BMP4 BMP2B | turquoise |
| | | DVR4 | |
| BOC | Brother of CDO (Protein BOC) | BOC | turquoise |
| | | UNQ604/PRO1190 | |
| BSG | Basigin (5F7) (Collagenase stimulatory | BSG | turquoise |
| | factor) (Ex | UNQ6505/PRO21383 | |
| BST2 | Bone marrow stromal antigen 2 (BST-2) | BST2 | turquoise |
| BTN2A1 | Butyrophilin subfamily 2 member A1 | BTN2A1 BT2.1 | turquoise |
| | | BTF1 | |
| BTN3A2 | Butyrophilin subfamily 3 member A2 | BTN3A2 BT3.2 | turquoise |
| | | BTF3 BTF4 | |
| C1QA | Complement C1q subcomponent subunit A | C1QA | turquoise |
| CA12 | Carbonic anhydrase 12 | CA12 | turquoise |
| CA14 | Carbonic anhydrase 14 | CA14 | turquoise |
| | | UNQ690/PRO1335 | |
| CA3 | Carbonic anhydrase 3 | CA3 | turquoise |
| CALB1 | Calbindin (Calbindin D28) (D-28K) | CALB1 CAB27 | turquoise |
| CALB2 | Calretinin (CR) (29 kDa calbindin) | CALB2 CAB29 | turquoise |
| CALCA | Calcitonin | CALCA CALC1 | turquoise |
| CASC4 | Cancer susceptibility candidate 4 | GOLM2 CASC4 | turquoise |
| | | UNQ2573/PRO6308 | |
| CCL14 | C-C motif chemokine 14 (Chemokine CC- | CCL14 NCC2 | turquoise |
| | 1/CC-3) | SCYA14 | |
| CCL15 | C-C motif chemokine 15 (Chemokine CC-2) | CCL15 MIP5 NCC3 | turquoise |
| | (HCC-2) | SCYA15 | |
| CCL18 | C-C motif chemokine 18 | CCL18 AMAC1 | turquoise |
| | | DCCK1 MIP4 | |
| | | PARC SCYA18 | |
| CCL19 | C-C motif chemokine 19 (Beta-chemokine | CCL19 ELC MIP3B | turquoise |
| | exodus-3) | SCYA19 | |
| CCL2 | C-C motif chemokine 2 (HC11) | CCL2 MCP1 | turquoise |
| | | SCYA2 | |
| CCL20 | C-C motif chemokine 20 (Beta-chemokine | CCL20 LARC | turquoise |
| | exodus-1) | MIP3A SCYA20 | |
| CCL23 | C-C motif chemokine 23 (CK-beta-8) | CCL23 MIP3 | turquoise |
| | (CKB-8) | MPIF1 SCYA23 | |
| CCL27 | C-C motif chemokine 27 (CC chemokine | CCL27 ILC | turquoise |
| | ILC) | SCYA27 | |
| CCL3 | C-C motif chemokine 3 | CCL3 GOS19-1 | turquoise |
| | | MIP1A SCYA3 | |
| CCN1 | CCN family member 1 | CCN1 CYR61 GIG1 | turquoise |
| | | IGFBP10 | |
| CCN3 | CCN family member 3 | CCN3 IGFBP9 | turquoise |
| | | NOV NOVH | |
| CD14 | Monocyte differentiation antigen CD14 | CD14 | turquoise |
| CD163 | Scavenger receptor cysteine-rich type 1 | CD163 M130 | turquoise |
| | protein M1 | | |
| CD200 | OX-2 membrane glycoprotein (CD antigen | CD200 MOX1 | turquoise |
| | CD200) | MOX2 My033 | |
| CD209 | CD209 antigen (C-type lectin domain | CD209 CLEC4L | turquoise |
| | family 4 member) | | |
| CD274 | Programmed cell death 1 ligand 1 (PD-L1) | CD274 B7H1 | turquoise |
| | | PDCD1L1 | |
| | | PDCD1LG1 PDL1 | |
| CD276 | CD276 antigen (4Ig-B7-H3) (B7 homolog | CD276 B7H3 | turquoise |
| | 3) (B7-H3) | PSEC0249 | |
| | | UNQ309/PRO352 | |
| CD28 | T-cell-specific surface glycoprotein CD28 | CD28 | turquoise |
| | (TP44) | | |
| CD300C | CMRF35-like molecule 6 (CLM-6) | CD300C CMRF35 | turquoise |
| | | CMRF35A | |
| | | CMRF35A1 IGSF16 | |
| CD300E | CMRF35-like molecule 2 (CLM-2) | CD300E CD300LE | turquoise |
| | | CLM2 CMRF35A5 | |
| | | IREM2 | |
| CD300LF | CMRF35-like molecule 1 (CLM-1) | CD300LF CD300F | turquoise |
| | | CLM1 IGSF13 | |
| | | IREM1 NKIR | |
| | | UNQ3105/PRO10111 | |
| CD300LG | CMRF35-like molecule 9 (CLM-9) | CD300LG CLM9 | turquoise |
| | | TREM4 | |
| | | UNQ422/PRO846 | |
| CD302 | CD302 antigen (C-type lectin BIMLEC) | CD302 CLEC13A | turquoise |
| | | DCL1 KIAA0022 | |
| CD34 | Hematopoietic progenitor cell antigen CD34 | CD34 | turquoise |
| CD38 | ADP-ribosyl cyclase/cyclic ADP-ribose | CD38 | turquoise |
| | hydrolase 1 | | |
| CD4 | T-cell surface glycoprotein CD4 | CD4 | turquoise |
| CD46 | Membrane cofactor protein (TLX) | CD46 MCP MIC10 | turquoise |
| CD48 | CD48 antigen | CD48 BCM1 | turquoise |
| | | BLAST1 | |
| CD5 | T-cell surface glycoprotein CD5 | CD5 LEU1 | turquoise |
| CD55 | Complement decay-accelerating factor | CD55 CR DAF | turquoise |
| CD58 | Lymphocyte function-associated antigen 3 | CD58 LFA3 | turquoise |
| | (Ag3) | | |
| CD59 | CD59 glycoprotein (1F5 antigen) | CD59 MIC11 MIN1 | turquoise |
| | | MIN2 MIN3 | |
| | | MSK21 | |
| CD74 | CD74 molecule | CD74 DHLAG | turquoise |
| CD93 | CD93 molecule | CD93 C1QR1 | turquoise |
| | | MXRA4 | |
| CD97 | Adhesion G protein-coupled receptor E5 | ADGRE5 CD97 | turquoise |
| CD99 | CD99 antigen (12E7) (E2 antigen) | CD99 MIC2 MIC2X | turquoise |
| | | MIC2Y | |
| CD99L2 | CD99 antigen-like protein 2 (MIC2-like | CD99L2 MIC2L1 | turquoise |
| | protein 1) | UNQ1964/PRO4486 | |
| CDCP1 | CUB domain-containing protein 1 | CDCP1 TRASK | turquoise |
| | | UNQ2486/PRO5773 | |
| CDH1 | Cadherin-1 (CAM 120/80) (Epithelial | CDH1 CDHE UVO | turquoise |
| | cadherin) | | |
| CDH15 | Cadherin-15 (Cadherin-14) (Muscle | CDH15 CDH14 | turquoise |
| | cadherin) | CDH3 | |
| CDH2 | Cadherin-2 (CDw325) (Neural cadherin) | CDH2 CDHN | turquoise |
| | (N-cadherin) | NCAD | |
| CDH3 | Cadherin-3 (Placental cadherin) (P- | CDH3 CDHP | turquoise |
| | cadherin) | | |
| CDH5 | Cadherin-5 (7B4 antigen) | CDH5 | turquoise |
| CDH6 | Cadherin-6 (Kidney cadherin) (K-cadherin) | CDH6 | turquoise |
| CDHR2 | Cadherin-related family member 2 | CDHR2 PCDH24 | turquoise |
| | | PCLKC | |
| CDKN1A | Cyclin-dependent kinase inhibitor 1 | CDKN1A CAP20 | turquoise |
| | | CDKN1 CIP1 | |
| | | MDA6 PIC1 SDI1 | |
| | | WAF1 | |
| CDNF | Cerebral dopamine neurotrophic factor | CDNF ARMETL1 | turquoise |
| CDSN | Corneodesmosin (S protein) | CDSN | turquoise |
| CEACAM1 | Carcinoembryonic antigen-related cell | CEACAM1 BGP | turquoise |
| | adhesion molecule 1 | BGP1 | |
| CEACAM3 | Carcinoembryonic antigen-related cell | CEACAM3 CD66D | turquoise |
| | adhesion molecule 3 | CGM1 | |
| CFC1 | Cryptic protein (Cryptic family protein 1) | CFC1 | turquoise |
| CGREF1 | Cell growth regulator with EF hand domain | CGREF1 CGR11 | turquoise |
| | protein | | |
| CHGB | Secretogranin-1 (Chromogranin-B) (CgB) | CHGB SCG1 | turquoise |
| CHI3L1 | Chitinase-3-like protein 1 | CHI3L1 | turquoise |
| CHRDL1 | Chordin-like protein 1 (Neuralin-1) | CHRDL1 NRLN1 | turquoise |
| CHRDL2 | Chordin-like protein 2 | CHRDL2 BNF1 | turquoise |
| | | CHL2 | |
| | | UNQ765/PRO1557 | |
| CKAP4 | Cytoskeleton-associated protein 4 | CKAP4 | turquoise |
| CLEC10A | C-type lectin domain family 10 member A | CLEC10A | turquoise |
| | | CLECSF13 | |
| | | CLECSF14 HML | |
| CLEC14A | C-type lectin domain family 14 member A | CLEC14A C14orf27 | turquoise |
| | | EGFR5 | |
| | | UNQ236/PRO269 | |
| CLEC1A | C-type lectin domain family 1 member A | CLEC1A CLEC1 | turquoise |
| | | UNQ569/PRO1131 | |
| CLEC7A | C-type lectin domain family 7 member A | CLEC7A BGR | turquoise |
| | | CLECSF12 | |
| | | DECTIN1 | |
| | | UNQ539/PRO1082 | |
| CLMP | CXADR-like membrane protein | CLMP ACAM | turquoise |
| | | ASAM | |
| | | UNQ318/PRO363 | |
| CLSTN2 | Calsyntenin-2 (Alcadein-gamma) (Alc- | CLSTN2 CS2 | turquoise |
| | gamma) | | |
| CNTN2 | Contactin-2 (Axonal glycoprotein TAG-1) | CNTN2 AXT TAG1 | turquoise |
| | (Axonin-1) | TAX1 | |
| CNTNAP2 | Contactin-associated protein-like 2 | CNTNAP2 CASPR2 | turquoise |
| | | KIAA0868 | |
| COL18A1 | Collagen alpha-1(XVIII) chain | COL18A1 | turquoise |
| COL1A1 | Collagen alpha-1(I) chain | COL1A1 | turquoise |
| COL4A1 | Collagen alpha-1(IV) chain | COL4A1 | turquoise |
| COL6A3 | Collagen alpha-3(VI) chain | COL6A3 | turquoise |
| COLEC12 | Collectin-12 (Collectin placenta protein 1) | COLEC12 CLP1 | turquoise |
| | | NSR2 SCARA4 | |
| | | SRCL | |
| COMP | Cartilage oligomeric matrix protein | COMP | turquoise |
| | (COMP) | | |
| CRELD2 | Protein disulfide isomerase CRELD2 | CRELD2 | turquoise |
| | | UNQ185/PRO211 | |
| CRIM1 | Cysteine-rich motor neuron 1 protein | CRIM1 S52 | turquoise |
| | (CRIM-1) | UNQ1886/PRO4330 | |
| CRIP2 | Cysteine-rich protein 2 (CRP-2) (Protein | CRIP2 CRP2 | turquoise |
| | ESP1) | | |
| CRLF1 | Cytokine receptor-like factor 1 | CRLF1 | turquoise |
| | | UNQ288/PRO327 | |
| CRTAM | Cytotoxic and regulatory T-cell molecule | CRTAM | turquoise |
| CSF1 | Macrophage colony-stimulating factor 1 | CSF1 | turquoise |
| | (CSF-1) | | |
| CSF2RA | Granulocyte-macrophage colony- | CSF2RA CSF2R | turquoise |
| | stimulating factor 2 receptor subunit alpha | CSF2RY | |
| CSF3 | Granulocyte colony-stimulating factor (G- | CSF3 C17orf33 | turquoise |
| | CSF) | GCSF | |
| CST3 | Cystatin-C (Cystatin-3) (Gamma-trace) | CST3 | turquoise |
| CST6 | Cystatin-M (Cystatin-6) (Cystatin-E) | CST6 | turquoise |
| CSTB | Cystatin-B (CPI-B) | CSTB CST6 STFB | turquoise |
| CTSB | Cathepsin B (EC 3.4.22.1) (APP secretase) | CTSB CPSB | turquoise |
| | (APPS) | | |
| CTSD | Cathepsin D | CTSD CPSD | turquoise |
| CTSL | Procathepsin L | CTSL CTSL1 | turquoise |
| CTSV | Cathepsin L2 (EC 3.4.22.43) (Cathepsin U) | CTSV CATL2 | turquoise |
| | | CTSL2 CTSU | |
| | | UNQ268/PRO305 | |
| CTSZ | Cathepsin Z (EC 3.4.18.1) (Cathepsin P) | CTSZ | turquoise |
| CX3CL1 | Fractalkine (C-X3-C motif chemokine 1) | CX3CL1 FKN NTT | turquoise |
| | | SCYD1 A-152E5.2 | |
| CXADR | Coxsackievirus and adenovirus receptor | CXADR CAR | turquoise |
| | (CAR) | | |
| CXCL10 | C-X-C motif chemokine 10 | CXCL10 INP10 | turquoise |
| | | SCYB10 | |
| CXCL16 | C-X-C motif chemokine 16 | CXCL16 SCYB16 | turquoise |
| | | SRPSOX | |
| | | UNQ2759/PRO6714 | |
| CXCL17 | C-X-C motif chemokine 17 | CXCL17 VCC1 | turquoise |
| | | UNQ473/PRO842 | |
| DCBLD2 | Discoidin, CUB and LCCL domain- | DCBLD2 CLCP1 | turquoise |
| | containing protein | ESDN | |
| DCN | Decorin (Bone proteoglycan II) (PG-S2) | DCN SLRR1B | turquoise |
| | (PG40) | | |
| DDR1 | Epithelial discoidin domain-containing | DDR1 CAK | turquoise |
| | receptor 1 | EDDR1 NEP | |
| | | NTRK4 PTK3A | |
| | | RTK6 TRKE | |
| DKK3 | Dickkopf-related protein 3 (Dickkopf-3) | DKK3 REIC | turquoise |
| | (Dkk-3) | UNQ258/PRO295 | |
| DKK4 | Dickkopf-related protein 4 (Dickkopf-4) | DKK4 | turquoise |
| | (Dkk-4) | | |
| DLK1 | Protein delta homolog 1 (DLK-1) (pG2) | DLK1 DLK | turquoise |
| DLL1 | Delta-like protein 1 (Drosophila Delta | DLL1 | turquoise |
| | homolog 1) | UNQ146/PRO172 | |
| DPEP2 | Dipeptidase 2 | DPEP2 | turquoise |
| | | UNQ284/PRO323 | |
| DPT | Dermatopontin | DPT | turquoise |
| DRAXIN | Draxin (Dorsal inhibitory axon guidance | DRAXIN C1orf187 | turquoise |
| | protein) | PSEC0258 | |
| | | UNQ3119/PRO10268 | |
| DSC2 | Desmocollin-2 (Cadherin family member 2) | DSC2 CDHF2 | turquoise |
| | | DSC3 | |
| DSG2 | Desmoglein-2 (Cadherin family member 5) | DSG2 CDHF5 | turquoise |
| | (HDGC) | | |
| DSG3 | Desmoglein-3 (130 kDa pemphigus vulgaris | DSG3 CDHF6 | turquoise |
| | antigen) | | |
| ECE1 | Endothelin-converting enzyme 1 (ECE-1) | ECE1 | turquoise |
| EDA2R | Tumor necrosis factor receptor superfamily | EDA2R TNFRSF27 | turquoise |
| | member | XEDAR | |
| | | UNQ2448/PRO5727/ | |
| | | PRO34080 | |
| EFEMP1 | EGF-containing fibulin-like extracellular | EFEMP1 FBLN3 | turquoise |
| | matrix p | FBNL | |
| EFNA1 | Ephrin-A1 | EFNA1 EPLG1 | turquoise |
| | | LERK1 TNFAIP4 | |
| EFNA4 | Ephrin-A4 | EFNA4 EPLG4 | turquoise |
| | | LERK4 | |
| ENAH | Protein enabled homolog | ENAH MENA | turquoise |
| ENG | Endoglin (CD antigen CD105) | ENG END | turquoise |
| ENPP2 | Ectonucleotide | ENPP2 ATX | turquoise |
| | pyrophosphatase/phosphodiesterase 2 | PDNP2 | |
| ENTPD2 | Ectonucleoside triphosphate | ENTPD2 CD39L1 | turquoise |
| | diphosphohydrolase 2 | | |
| ENTPD6 | Ectonucleoside triphosphate | ENTPD6 CD39L2 | turquoise |
| | diphosphohydrolase 6 | IL6ST2 | |
| EPHA1 | Ephrin type-A receptor 1 (hEpha1) | EPHA1 EPH EPHT | turquoise |
| | | EPHT1 | |
| EPHA2 | Ephrin type-A receptor 2 | EPHA2 ECK | turquoise |
| EPHB4 | Ephrin type-B receptor 4 | EPHB4 HTK | turquoise |
| | | MYK1 TYRO11 | |
| EPHB6 | Ephrin type-B receptor 6 (HEP) | EPHB6 | turquoise |
| EPS8L2 | Epidermal growth factor receptor kinase | EPS8L2 EPS8R2 | turquoise |
| | substrate | PP13181 | |
| ERBB3 | Receptor tyrosine-protein kinase erbB-3 | ERBB3 HER3 | turquoise |
| ESAM | Endothelial cell-selective adhesion molecule | ESAM | turquoise |
| | | UNQ220/PRO246 | |
| F2R | Proteinase-activated receptor 1 (PAR-1) | F2R CF2R PAR1 | turquoise |
| | | TR | |
| F3 | Tissue factor (TF) (Coagulation factor III) | F3 | turquoise |
| FABP4 | Fatty acid-binding protein, adipocyte | FABP4 | turquoise |
| FAM3B | Protein FAM3B (Cytokine-like protein 2- | FAM3B C21orf11 | turquoise |
| | 21) | C21orf76 PRED44 | |
| | | UNQ320/PRO365 | |
| FAM3C | Protein FAM3C (Interleukin-like EMT | FAM3C ILEI | turquoise |
| | inducer) | GS3786 | |
| FAS | Tumor necrosis factor receptor superfamily | FAS APT1 FAS1 | turquoise |
| | member | TNFRSF6 | |
| FCRLB | Fc receptor-like B | FCRLB FCRL2 | turquoise |
| | | FCRLM2 FCRY | |
| | | FREB2 | |
| FGF21 | Fibroblast growth factor 21 (FGF-21) | FGF21 | turquoise |
| | | UNQ3115/PRO10196 | |
| FGF23 | Fibroblast growth factor 23 (FGF-23) | FGF23 HYPF | turquoise |
| | | UNQ3027/PRO9828 | |
| FGF5 | Fibroblast growth factor 5 (FGF-5) | FGF5 | turquoise |
| FGFR2 | Fibroblast growth factor receptor 2 (FGFR- | FGFR2 BEK KGFR | turquoise |
| | 2) | KSAM | |
| FLRT2 | Leucine-rich repeat transmembrane protein | FLRT2 KIAA0405 | turquoise |
| | FLRT2 | UNQ232/PRO265 | |
| FLT1 | Vascular endothelial growth factor receptor | FLT1 FLT FRT | turquoise |
| | 1 | VEGFR1 | |
| FLT3LG | Fms-related tyrosine kinase 3 ligand (Flt3 | FLT3LG | turquoise |
| | ligand) | | |
| FOLR1 | Folate receptor alpha (FR-alpha) | FOLR1 FOLR | turquoise |
| FOLR2 | Folate receptor beta (FR-beta) (Folate | FOLR2 | turquoise |
| | receptor 2) | | |
| FOLR3 | Folate receptor gamma (FR-gamma) (Folate | FOLR3 | turquoise |
| | receptor 3) | | |
| FST | Follistatin (FS) (Activin-binding protein) | FST | turquoise |
| FSTL3 | Follistatin-related protein 3 | FSTL3 FLRG | turquoise |
| | | UNQ674/PRO1308 | |
| GALNT10 | Polypeptide N- | GALNT10 | turquoise |
| | acetylgalactosaminyltransferase 10 | | |
| GALNT2 | Polypeptide N- | GALNT2 | turquoise |
| | acetylgalactosaminyltransferase 2 | | |
| GAS6 | Growth arrest-specific protein 6 (GAS-6) | GAS6 AXLLG | turquoise |
| GCNT1 | Beta-1,3-galactosyl-O-glycosyl- | GCNT1 NACGT2 | turquoise |
| | glycoprotein beta-1 | | |
| GDF15 | Growth/differentiation factor 15 (GDF-15) | GDF15 MIC1 PDF | turquoise |
| | | PLAB PTGFB | |
| GDNF | Glial cell line-derived neurotrophic factor | GDNF | turquoise |
| GFAP | Glial fibrillary acidic protein (GFAP) | GFAP | turquoise |
| GFRA1 | GDNF family receptor alpha-1 | GFRA1 GDNFRA | turquoise |
| | | RETL1 TRNR1 | |
| GFRA2 | GDNF family receptor alpha-2 | GFRA2 GDNFRB | turquoise |
| | | RETL2 TRNR2 | |
| GFRA3 | GDNF family receptor alpha-3 | GFRA3 | turquoise |
| | | UNQ339/PRO538/ | |
| | | PRO3664 | |
| GGT1 | Glutathione hydrolase 1 proenzyme | GGT1 GGT | turquoise |
| GGT5 | Glutathione hydrolase 5 proenzyme | GGT5 GGTLA1 | turquoise |
| GHRL | Appetite-regulating hormone | GHRL MTLRP | turquoise |
| | | UNQ524/PRO1066 | |
| GKN1 | Gastrokine-1 (18 kDa antrum mucosa | GKN1 AMP18 | turquoise |
| | protein) | CA11 | |
| | | UNQ489/PRO1005 | |
| GPC1 | Glypican-1 [Cleaved into: Secreted | GPC1 | turquoise |
| | glypican-1] | | |
| GPNMB | Transmembrane glycoprotein NMB | GPNMB HGFIN | turquoise |
| | | NMB | |
| | | UNQ1725/PRO9925 | |
| GPR37 | G protein coupled receptor 37 | GPR37 | turquoise |
| GUCAZA | Guanylin (Guanylate cyclase activator 2A) | GUCA2A GUCA2 | turquoise |
| HAVCR1 | Hepatitis A virus cellular receptor 1 | HAVCR1 KIM1 | turquoise |
| | (HAVcr-1) | TIM1 TIMD1 | |
| HAVCR2 | Hepatitis A virus cellular receptor 2 | HAVCR2 TIM3 | turquoise |
| | (HAVcr-2) | TIMD3 | |
| HLA-E | Major histocompatibility complex class I, E | HLA-E HLA-6.2 | turquoise |
| | | HLAE | |
| HS3ST3B1 | Heparan sulfate glucosamine 3-O- | HS3ST3B1 | turquoise |
| | sulfotransferase 3 | 3OST3B1 HS3ST3B | |
| HS6ST1 | Heparan-sulfate 6-O-sulfotransferase 1 | HS6ST1 HS6ST | turquoise |
| | (HS6ST-1) | | |
| HSPB6 | Heat shock protein beta-6 (HspB6) | HSPB6 | turquoise |
| HSPG2 | heparan sulfate proteoglycan 2 | HSPG2 | turquoise |
| HYAL1 | Hyaluronidase-1 (Hyal-1) (EC 3.2.1.35) | HYAL1 LUCA1 | turquoise |
| HYOU1 | Hypoxia up-regulated protein 1 | HYOU1 GRP170 | turquoise |
| | | ORP150 | |
| ICAM1 | Intercellular adhesion molecule 1 (ICAM-1) | ICAM1 | turquoise |
| ICAM2 | Intercellular adhesion molecule 2 (ICAM-2) | ICAM2 | turquoise |
| ICOSLG | ICOS ligand (B7 homolog 2) (B7-H2) | ICOSLG B7H2 | turquoise |
| | | B7RP1 ICOSL | |
| | | KIAA0653 | |
| IFNGR1 | Interferon gamma receptor 1 (IFN-gamma | IFNGR1 | turquoise |
| | receptor 1) | | |
| IFNL1 | Interferon lambda-1 (IFN-lambda-1) | IFNL1 IL29 | turquoise |
| | | ZCYTO21 | |
| IFNLR1 | Interferon lambda receptor 1 | IFNLR1 IL28RA | turquoise |
| | | LICR2 | |
| IGF1R | Insulin-like growth factor 1 receptor | IGF1R | turquoise |
| IGF2R | Cation-independent mannose-6-phosphate | IGF2R MPRI | turquoise |
| | receptor | | |
| IGFBP1 | Insulin-like growth factor-binding protein 1 | IGFBP1 IBP1 | turquoise |
| IGFBP2 | Insulin-like growth factor-binding protein 2 | IGFBP2 BP2 IBP2 | turquoise |
| IGFBP3 | Insulin-like growth factor-binding protein 3 | IGFBP3 IBP3 | turquoise |
| IGFBP4 | Insulin-like growth factor-binding protein 4 | IGFBP4 IBP4 | turquoise |
| IGFBP6 | Insulin-like growth factor-binding protein 6 | IGFBP6 IBP6 | turquoise |
| IGFBP7 | Insulin-like growth factor-binding protein 7 | IGFBP7 MAC25 | turquoise |
| | | PSF | |
| IGFBPL1 | Insulin-like growth factor-binding protein- | IGFBPL1 | turquoise |
| | like 1 | IGFBPRP4 | |
| IGSF8 | Immunoglobulin superfamily member 8 | IGSF8 CD81P3 | turquoise |
| | (IgSF8) | EWI2 KCT4 | |
| IL10 | Interleukin-10 (IL-10) | IL10 | turquoise |
| IL10RB | Interleukin-10 receptor subunit beta | IL10RB CRFB4 | turquoise |
| | | D21S58 D21S66 | |
| IL12B | Interleukin-12 subunit beta (IL-12B) | IL12B NKSF2 | turquoise |
| IL12RB1 | Interleukin-12 receptor subunit beta-1 | IL12RB1 IL12R | turquoise |
| | | IL12RB | |
| IL13RA1 | Interleukin-13 receptor subunit alpha-1 | IL13RA1 IL13R | turquoise |
| | | IL13RA | |
| IL15 | Interleukin-15 (IL-15) | IL15 | turquoise |
| IL15RA | Interleukin-15 receptor subunit alpha | IL15RA | turquoise |
| IL17C | Interleukin-17C (IL-17C) (Cytokine CX2) | IL17C | turquoise |
| | | UNQ561/PRO1122 | |
| IL17D | Interleukin-17D (IL-17D) (Interleukin-27) | IL17D | turquoise |
| | (IL-27) | UNQ3096/PRO21175 | |
| IL17RB | Interleukin-17 receptor B (IL-17 receptor B) | IL17RB CRL4 | turquoise |
| | | EVI27 IL17BR | |
| | | UNQ2501/PRO19612 | |
| IL18 | Interleukin-18 (IL-18) (Iboctadekin) | IL18 IGIF IL1F4 | turquoise |
| IL18BP | Interleukin-18-binding protein (IL-18BP) | IL18BP | turquoise |
| IL18R1 | Interleukin-18 receptor 1 (IL-18R-1) (IL- | IL18R1 IL1RRP | turquoise |
| | 18R1) | | |
| IL19 | Interleukin-19 (IL-19) | IL19 ZMDA1 | turquoise |
| IL1R1 | Interleukin-1 receptor type 1 (IL-1R-1) (IL- | IL1R1 IL1R IL1RA | turquoise |
| | 1RT-1) | IL1RT1 | |
| IL1RL2 | Interleukin-1 receptor-like 2 | IL1RL2 IL1RRP2 | turquoise |
| IL22RA1 | Interleukin-22 receptor subunit alpha-1 | IL22RA1 IL22R | turquoise |
| IL33 | Interleukin-33 (IL-33) | IL33 C9orf26 | turquoise |
| | | IL1F11 NFHEV | |
| IL3RA | Interleukin-3 receptor subunit alpha | IL3RA IL3R | turquoise |
| ILAR | Interleukin-4 receptor subunit alpha | IL4R IL4RA | turquoise |
| | | 582J2.1 | |
| IL6ST | Interleukin-6 receptor subunit beta | IL6ST | turquoise |
| IL7 | Interleukin-7 (IL-7) | IL7 | turquoise |
| ISM1 | Isthmin-1 | ISM1 C20orf82 ISM | turquoise |
| ITGA5 | Integrin alpha-5 | ITGA5 FNRA | turquoise |
| ITGB1 | Integrin beta-1 | ITGB1 FNRB | turquoise |
| | | MDF2 MSK12 | |
| ITGB6 | Integrin beta-6 | ITGB6 | turquoise |
| ITIH3 | Inter-alpha-trypsin inhibitor heavy chain H3 | ITIH3 | turquoise |
| JAM2 | Junctional adhesion molecule B (JAM-B) | JAM2 C21orf43 | turquoise |
| | | VEJAM | |
| | | UNQ219/PRO245 | |
| KAZALD1 | Kazal-type serine protease inhibitor domain | KAZALD1 FKSG28 | turquoise |
| | 1 | FKSG40 | |
| | | UNQ2945/PRO21184 | |
| KLK10 | Kallikrein-10 | KLK10 NES1 | turquoise |
| | | PRSSL1 | |
| KLK11 | Kallikrein-11 (hK11) | KLK11 PRSS20 | turquoise |
| | | TLSP | |
| | | UNQ649/PRO1279 | |
| KLK13 | Kallikrein-13 | KLK13 KLKL4 | turquoise |
| KLK4 | Kallikrein-4 | KLK4 EMSP1 | turquoise |
| | | PRSS17 PSTS | |
| KLK6 | Kallikrein-6 | KLK6 PRSS18 | turquoise |
| | | PRSS9 | |
| KLK8 | Kallikrein-8 (hK8) | KLK8 NRPN | turquoise |
| | | PRSS19 TADG14 | |
| | | UNQ283/PRO322 | |
| KRT19 | Keratin, type I cytoskeletal 19 (Cytokeratin- | KRT19 | turquoise |
| | 19) | | |
| KRT5 | Keratin, type II cytoskeletal 5 | KRT5 | turquoise |
| LAG3 | Lymphocyte activation gene 3 protein | LAG3 FDC | turquoise |
| | (LAG-3) | | |
| LAIR1 | Leukocyte-associated immunoglobulin-like | LAIR1 CD305 | turquoise |
| | receptor 1 | | |
| LAIR2 | Leukocyte-associated immunoglobulin-like | LAIR2 CD306 | turquoise |
| | receptor 2 | | |
| LAMA4 | Laminin subunit alpha-4 (Laminin-14 | LAMA4 | turquoise |
| | subunit alpha) | | |
| LAMP3 | Lysosome-associated membrane | LAMP3 DCLAMP | turquoise |
| | glycoprotein 3 | TSC403 | |
| LAYN | Layilin | LAYN | turquoise |
| | | UNQ208/PRO234 | |
| LGALS1 | Galectin-1 (Gal-1) | LGALS1 | turquoise |
| LGALS3 | Galectin-3 (Gal-3) (35 kDa lectin) | LGALS3 MAC2 | turquoise |
| LGALS4 | Galectin-4 (Gal-4) (Antigen NY-CO-27) | LGALS4 | turquoise |
| LGALS7_LGALS7B | Galectin-7 (Gal-7) (HKL-14) (PI7) | LGALS7 PIG1; | turquoise |
| LGALS9 | Galectin-9 (Gal-9) (Ecalectin) | LGALS9 | turquoise |
| LIFR | Leukemia inhibitory factor receptor (LIF | LIFR | turquoise |
| | receptor) | | |
| LILRA2 | Leukocyte immunoglobulin-like receptor | LILRA2 ILT1 LIR7 | turquoise |
| | subfamily A2 | | |
| LILRA5 | Leukocyte immunoglobulin-like receptor | LILRA5 ILT11 | turquoise |
| | subfamily A5 | LILRB7 LIR9 | |
| LILRB1 | Leukocyte immunoglobulin-like receptor | LILRB1 ILT2 LIR1 | turquoise |
| | subfamily B1 | MIR7 | |
| LILRB2 | Leukocyte immunoglobulin-like receptor | LILRB2 ILT4 LIR2 | turquoise |
| | subfamily B2 | MIR10 | |
| LILRB4 | Leukocyte immunoglobulin-like receptor | LILRB4 ILT3 LIR5 | turquoise |
| | subfamily B4 | | |
| LRP1 | Prolow-density lipoprotein receptor-related | LRP1 A2MR APR | turquoise |
| | protein 1 | | |
| LRP11 | Low-density lipoprotein receptor-related | LRP11 | turquoise |
| | protein 11 | | |
| LRRC25 | Leucine-rich repeat-containing protein 25 | LRRC25 MAPA | turquoise |
| | | UNQ6169/PRO20174 | |
| LTBP2 | Latent-transforming growth factor beta- | LTBP2 C14orf141 | turquoise |
| | binding pro | LTBP3 | |
| LTBP3 | Latent-transforming growth factor beta- | LTBP3 | turquoise |
| | binding protein 2 | | |
| LTBR | Lymphotoxin beta receptor | LTBR D12S370 | turquoise |
| | | TNFCR TNFR3 | |
| | | TNFRSF3 | |
| LY6D | Lymphocyte antigen 6D (Ly-6D) (E48 | LY6D E48 | turquoise |
| | antigen) | | |
| LY96 | Lymphocyte antigen 96 (Ly-96) (ESOP-1) | LY96 ESOP1 MD2 | turquoise |
| MANSC1 | MANSC domain-containing protein 1 | MANSC1 | turquoise |
| | | LOH12CR3 | |
| | | UNQ316/PRO361 | |
| MARCO | Macrophage receptor MARCO | MARCO SCARA2 | turquoise |
| MATN2 | Matrilin-2 | MATN2 | turquoise |
| | | UNQ193/PRO219 | |
| MATN3 | Matrilin-3 | MATN3 | turquoise |
| MB | Myoglobin | MB | turquoise |
| MCAM | Cell surface glycoprotein MUC18 | MCAM MUC18 | turquoise |
| MCFD2 | Multiple coagulation factor deficiency | MCFD2 SDNSF | turquoise |
| | protein 2 | | |
| MEPE | Matrix extracellular phosphoglycoprotein | MEPE | turquoise |
| MERTK | Tyrosine-protein kinase Mer | MERTK MER | turquoise |
| MET | Hepatocyte growth factor receptor (HGF | MET | turquoise |
| | receptor) | | |
| MFAP5 | Microfibrillar-associated protein 5 (MFAP- | MFAP5 MAGP2 | turquoise |
| | 5) | | |
| MIA | Melanoma-derived growth regulatory | MIA | turquoise |
| | protein | | |
| MILR1 | Allergin-1 (Allergy inhibitory receptor 1) | MILR1 C17orf60 | turquoise |
| | | MCA32 | |
| MLN | Promotilin | MLN | turquoise |
| MMP10 | Stromelysin-2 (SL-2) | MMP10 STMY2 | turquoise |
| MMP13 | Collagenase 3 | MMP13 | turquoise |
| MMP7 | Matrilysin (EC 3.4.24.23) (Matrin) | MMP7 MPSL1 | turquoise |
| | | PUMP1 | |
| MOG | Myelin-oligodendrocyte glycoprotein | MOG | turquoise |
| MSMB | Beta-microseminoprotein | MSMB PRSP | turquoise |
| MSR1 | Macrophage scavenger receptor types I and | MSR1 SCARA1 | turquoise |
| | II | | |
| MUC13 | Mucin-13 (MUC-13) | MUC13 DRCC1 | turquoise |
| | | REC | |
| | | UNQ6194/PRO20221 | |
| MYOC | Myocilin (Myocilin 55 kDa subunit) | MYOC GLC1A | turquoise |
| | | TIGR | |
| NBL1 | Neuroblastoma suppressor of tumorigenicity | NBL1 DAN | turquoise |
| | 1 | DAND1 | |
| NCR1 | Natural cytotoxicity triggering receptor 1 | NCR1 LY94 | turquoise |
| NCS1 | Neuronal calcium sensor 1 (NCS-1) | NCS1 FLUP FREQ | turquoise |
| NECTIN2 | Nectin-2 (Herpes virus entry mediator B) | NECTIN2 HVEB | turquoise |
| | | PRR2 PVRL2 | |
| NECTIN4 | Nectin-4 (Ig superfamily receptor LNIR) | NECTIN4 LNIR | turquoise |
| | | PRR4 PVRL4 | |
| NELL1 | Protein kinase C-binding protein NELL1 | NELL1 NRP1 | turquoise |
| NELL2 | Protein kinase C-binding protein NELL2 | NELL2 NRP2 | turquoise |
| NFASC | Neurofascin | NFASC KIAA0756 | turquoise |
| NOMO1 | Nodal modulator 1 (pM5 protein) | NOMO1 PM5 | turquoise |
| NOS1 | Nitric oxide synthase 1 | NOS1 | turquoise |
| NOTCH1 | Neurogenic locus notch homolog protein 1 | NOTCH1 TAN1 | turquoise |
| | (Notch 1) | | |
| NOTCH3 | Neurogenic locus notch homolog protein 3 | NOTCH3 | turquoise |
| | (Notch 3) | | |
| NPDC1 | Neural proliferation differentiation and | NPDC1 | turquoise |
| | control 1 | | |
| NPPB | Natriuretic peptides B | NPPB | turquoise |
| NPPC | C-type natriuretic peptide | NPPC CNP2 | turquoise |
| NRCAM | Neuronal cell adhesion molecule (Nr-CAM) | NRCAM KIAA0343 | turquoise |
| NRP1 | Neuropilin-1 | NRP1 NRP | turquoise |
| | | VEGF165R | |
| NRP2 | Neuropilin-2 | NRP2 VEGF165R2 | turquoise |
| NT5E | 5′-nucleotidase (5′-NT) | NT5E NT5 NTE | turquoise |
| NUCB2 | Nucleobindin-2 (DNA-binding protein | NUCB2 NEFA | turquoise |
| | NEFA) | | |
| NXPH1 | Neurexophilin-1 | NXPH1 NPH1 | turquoise |
| | | Nbla00697 | |
| OGN | Mimecan (Osteoglycin) (Osteoinductive | OGN OIF SLRR3A | turquoise |
| | factor) | | |
| OPTC | Opticin (Oculoglycan) | OPTC OPT | turquoise |
| OSCAR | Osteoclast-associated immunoglobulin-like | OSCAR | turquoise |
| | receptor | | |
| OSMR | Oncostatin-M-specific receptor subunit beta | OSMR OSMRB | turquoise |
| OXT | Oxytocin-neurophysin 1 (OT-NPI) | OXT OT | turquoise |
| PAM | Peptidyl-glycine alpha-amidating | PAM | turquoise |
| | monooxygenase | | |
| PCDH1 | Protocadherin-1 (Cadherin-like protein 1) | PCDH1 | turquoise |
| PCDH17 | Protocadherin-17 (Protocadherin-68) | PCDH17 PCDH68 | turquoise |
| | | PCH68 | |
| PCSK9 | Proprotein convertase subtilisin/kexin type 9 | PCSK9 NARC1 | turquoise |
| | | PSEC0052 | |
| PDCD1 | Programmed cell death protein 1 (Protein | PDCD1 PD1 | turquoise |
| | PD-1) | | |
| PDCD1LG2 | Programmed cell death 1 ligand 2 (PD-1 | PDCD1LG2 B7DC | turquoise |
| | ligand 2) | CD273 PDCD1L2 | |
| | | PDL2 | |
| PDGFC | Platelet-derived growth factor C (PDGF-C) | PDGFC SCDGF | turquoise |
| | | UNQ174/PRO200 | |
| PDGFRA | Platelet-derived growth factor receptor alpha | PDGFRA PDGFR2 | turquoise |
| | | RHEPDGFRA | |
| PDGFRB | Platelet-derived growth factor receptor beta | PDGFRB PDGFR | turquoise |
| | | PDGFR1 | |
| PGF | Placenta growth factor (PlGF) | PGF | turquoise |
| | | PGFL PLGF | |
| PHOSPHO1 | Phosphoethanolamine/phosphocholine | PHOSPHO1 | turquoise |
| | phosphatase | | |
| PI3 | Elafin (Elastase-specific inhibitor) (ESI) | PI3 WAP3 | turquoise |
| | | WFDC14 | |
| PIGR | Polymeric immunoglobulin receptor (PIgR) | PIGR | turquoise |
| PIK3IP1 | Phosphoinositide-3-kinase-interacting | PIK3IP1 HGFL | turquoise |
| | protein 1 | | |
| PILRA | Paired immunoglobulin-like type 2 receptor | PILRA | turquoise |
| | alpha | | |
| PILRB | Paired immunoglobulin-like type 2 receptor | PILRB FDFACT | turquoise |
| | beta | PP1551 | |
| PLA2G15 | Phospholipase A2 group XV | PLA2G15 LYPLA3 | turquoise |
| | | UNQ341/PRO540 | |
| PLAT | Tissue-type plasminogen activator (t-PA) | PLAT | turquoise |
| PLAUR | Urokinase plasminogen activator surface | PLAUR MO3 | turquoise |
| | receptor | UPAR | |
| PLIN1 | Perilipin-1 (Lipid droplet-associated | PLIN1 PERI PLIN | turquoise |
| | protein) | | |
| PLXNB2 | Plexin-B2 (MM1) | PLXNB2 | turquoise |
| | | KIAA0315 | |
| PODXL | Podocalyxin (GCTM-2 antigen) (Gp200) | PODXL PCLP | turquoise |
| | | PCLP1 | |
| PRELP | Prolargin | PRELP SLRR2A | turquoise |
| PRSS2 | Trypsin-2 | PRSS2 TRY2 | turquoise |
| | | TRYP2 | |
| PRSS8 | Prostasin | PRSS8 | turquoise |
| PRTG | Protogenin (Protein Shen-Dan) | PRTG | turquoise |
| PSG1 | Pregnancy-specific beta-1-glycoprotein 1 | PSG1 B1G1 PSBG1 | turquoise |
| | | PSGGA | |
| PTGDS | Prostaglandin-H2 D-isomerase | PTGDS PDS | turquoise |
| PTH1R | Parathyroid hormone/parathyroid hormone | PTHIR PTHR | turquoise |
| | receptor 1 | PTHR1 | |
| PTK7 | Inactive tyrosine-protein kinase 7 | PTK7 CCK4 | turquoise |
| PTPRF | Receptor-type tyrosine-protein phosphatase | PTPRF LAR | turquoise |
| | F | | |
| PTPRM | protein tyrosine phosphatase receptor type | PTPRM PTPRL1 | turquoise |
| | M | | |
| PTPRN2 | Receptor-type tyrosine-protein phosphatase | PTPRN2 KIAA0387 | turquoise |
| | N2 | | |
| PTPRS | Receptor-type tyrosine-protein phosphatase | PTPRS | turquoise |
| | S | | |
| PVR | Poliovirus receptor (Nectin-like protein 5) | PVR PVS | turquoise |
| RARRES1 | Retinoic acid receptor responder protein 1 | RARRES1 PEIG1 | turquoise |
| | | TIG1 | |
| RBP2 | Retinol-binding protein 2 | RBP2 CRBP2 | turquoise |
| REG1A | Regenerating family member alpha 1 | REG1A PSPS | turquoise |
| | | PSPS1 REG | |
| REG1B | Regenerating family member 1 beta | REG1B PSPS2 | turquoise |
| | | REGL | |
| REG3A | Regenerating islet-derived protein 3-alpha | REG3A HIP PAP | turquoise |
| | | PAP1 | |
| REG4 | Regenerating islet-derived protein 4 (REG- | REG4 GISP RELP | turquoise |
| | 4) | | |
| RELT | Tumor necrosis factor receptor superfamily | RELT TNFRSF19L | turquoise |
| | member | | |
| RGMA | Repulsive guidance molecule A | RGMA RGM | turquoise |
| RGMB | RGM domain family member B | RGMB | turquoise |
| RNASET2 | Ribonuclease T2 (EC 4.6.1.19) | RNASET2 | turquoise |
| | (Ribonuclease 6) | RNASE6PL | |
| ROBO1 | Roundabout homolog 1 | ROBO1 DUTT1 | turquoise |
| ROR1 | receptor tyrosine kinase like orphan receptor | ROR1 NTRKR1 | turquoise |
| | 1 | | |
| RSPO3 | R-spondin-3 (Protein with TSP type-1 | RSPO3 PWTSR | turquoise |
| | repeat) | THSD2 | |
| RTBDN | Retbindin | RTBDN | turquoise |
| RTN4R | Reticulon-4 receptor (Nogo receptor) (NgR) | RTN4R NOGOR | turquoise |
| | | UNQ330/PRO526 | |
| SCARA5 | Scavenger receptor class A member 5 | SCARA5 | turquoise |
| | | UNQ2938/PRO28700 | |
| SCARB2 | Scavenger receptor class B member 2 | SCARB2 CD36L2 | turquoise |
| | | LIMP2 LIMPII | |
| SCARF2 | Scavenger receptor class F member 2 | SCARF2 SREC2 | turquoise |
| | | SREPCR | |
| SCG2 | Secretogranin-2 (Chromogranin-C) | SCG2 CHGC | turquoise |
| SCG3 | Secretogranin-3 (Secretogranin III) (SgIII) | SCG3 | turquoise |
| | | UNQ2502/PRO5990 | |
| SCGB1A1 | secretoglobin family 1A member 1 | SCGB1A1 CC10 | turquoise |
| | | CCSP UGB | |
| SDC1 | Syndecan-1 (SYND1) (CD antigen CD138) | SDC1 SDC | turquoise |
| SEMA3F | Semaphorin-3F (Sema III/F) (Semaphorin | SEMA3F | turquoise |
| | IV) | | |
| SEMA7A | Semaphorin-7A (CDw108) | SEMA7A CD108 | turquoise |
| | | SEMAL | |
| SERPINB6 | Serpin B6 (Cytoplasmic antiproteinase) | SERPINB6 PI6 PTI | turquoise |
| | (CAP) | | |
| SEZ6L | Seizure 6-like protein | SEZ6L KIAA0927 | turquoise |
| | | UNQ2542/PRO6094 | |
| SEZ6L2 | Seizure 6-like protein 2 | SEZ6L2 PSK | turquoise |
| | | UNQ1903/PRO4349 | |
| SIGLEC1 | Sialoadhesin | SIGLEC1 SN | turquoise |
| SIGLEC10 | Sialic acid-binding Ig-like lectin 10 (Siglec- | SIGLEC10 SLG2 | turquoise |
| | 10) | UNQ477/PRO940 | |
| SIGLEC5 | Sialic acid-binding Ig-like lectin 5 (Siglec- | SIGLEC5 CD33L2 | turquoise |
| | 5) | OBBP2 | |
| SIGLEC7 | Sialic acid-binding Ig-like lectin 7 (Siglec- | SIGLEC7 AIRM1 | turquoise |
| | 7) (A | | |
| SIGLEC9 | Sialic acid-binding Ig-like lectin 9 (Siglec- | SIGLEC9 | turquoise |
| | 9) (C | UNQ668/PRO1302 | |
| SIRPA | Signal regulatory protein alpha | SIRPA BIT MFR | turquoise |
| | | MYD1 PTPNS1 | |
| | | SHPS1 SIRP | |
| SIRPB1 | Signal-regulatory protein beta-1 (SIRP-beta- | SIRPB1 | turquoise |
| | 1) | | |
| SLAMF8 | SLAM family member 8 | SLAMF8 BLAME | turquoise |
| SLC39A14 | solute carrier family 39 member 14 | SLC39A14 | turquoise |
| | | KIAA0062 ZIP14 | |
| SLC39A5 | solute carrier family 39 member 5 | SLC39A5 ZIP5 | turquoise |
| SLITRK2 | SLIT and NTRK-like protein 2 | SLITRK2 CXorf2 | turquoise |
| | | KIAA1854 SLITL1 | |
| | | UNQ9197/PRO34756 | |
| SLITRK6 | SLIT and NTRK-like protein 6 | SLITRK6 | turquoise |
| SMOC2 | SPARC-related modular calcium-binding | SMOC2 SMAP2 | turquoise |
| | protein 2 | MSTP117 | |
| SNCG | Gamma-synuclein | SNCG BCSG1 | turquoise |
| | | PERSYN PRSN | |
| SORCS2 | VPS10 domain-containing receptor SorCS2 | SORCS2 KIAA1329 | turquoise |
| SOST | Sclerostin | SOST | turquoise |
| | | UNQ2976/PRO7455/ | |
| | | PRO7476 | |
| SPARCL1 | SPARC-like protein 1 | SPARCL1 | turquoise |
| SPINK1 | Serine protease inhibitor Kazal-type 1 | SPINK1 PSTI | turquoise |
| SPINK4 | Serine protease inhibitor Kazal-type 4 | SPINK4 | turquoise |
| SPINK5 | Serine protease inhibitor Kazal-type 5 | SPINK5 | turquoise |
| SPINK6 | Serine protease inhibitor Kazal-type 6 | SPINK6 | turquoise |
| | | UNQ844/PRO1782 | |
| SPON1 | Spondin-1 (F-spondin) | SPON1 KIAA0762 | turquoise |
| | | VSGP | |
| SPON2 | Spondin-2 | SPON2 DIL1 | turquoise |
| | | UNQ435/PRO866 | |
| SPP1 | Osteopontin (Bone sialoprotein 1) | SPP1 BNSP OPN | turquoise |
| | (Nephropontin) | PSEC0156 | |
| ST6GAL1 | ST6 beta-galactoside alpha-2,6- | ST6GAL1 SIAT1 | turquoise |
| | sialyltransferase 1 | | |
| STC1 | Stanniocalcin-1 (STC-1) | STC1 STC | turquoise |
| STC2 | Stanniocalcin-2 (STC-2) | STC2 | turquoise |
| SUSD2 | Sushi domain-containing protein 2 | SUSD2 | turquoise |
| TACSTD2 | Tumor-associated calcium signal transducer | TACSTD2 GA733-1 | turquoise |
| | 2 | MIS1 TROP2 | |
| TAFA5 | Chemokine-like protein TAFA-5 | TAFA5 FAM19A5 | turquoise |
| | | UNQ5208/PRO34524 | |
| TCN2 | Transcobalamin-2 (TC-2) (Transcobalamin | TCN2 TC2 | turquoise |
| | II) | | |
| TEK | TEK receptor tyrosine kinase | TEK TIE2 VMCM | turquoise |
| | | VMCM1 | |
| TFF1 | Trefoil factor 1 | TFF1 BCEI PS2 | turquoise |
| TFF2 | Trefoil factor 2 (Spasmolysin) | TFF2 SML1 | turquoise |
| TFF3 | Trefoil factor 3 (Intestinal trefoil factor) | TFF3 ITF TFI | turquoise |
| TFPI | Tissue factor pathway inhibitor (TFPI) | TFPI LACI TFPI1 | turquoise |
| TGFBI | Transforming growth factor-beta-induced | TGFBI BIGH3 | turquoise |
| | protein | | |
| TGFBR2 | TGF-beta receptor type-2 (TGFR-2) | TGFBR2 | turquoise |
| TGFBR3 | Transforming growth factor beta receptor | TGFBR3 | turquoise |
| | type 3 | | |
| THBD | Thrombomodulin (TM) (Fetomodulin) | THBD THRM | turquoise |
| THBS2 | Thrombospondin-2 | THBS2 TSP2 | turquoise |
| THBS4 | Thrombospondin-4 | THBS4 TSP4 | turquoise |
| THY1 | Thy-1 membrane glycoprotein (CDw90) | THY1 | turquoise |
| TIE1 | Tyrosine-protein kinase receptor Tie-1 | TIE1 TIE | turquoise |
| TIMD4 | T-cell immunoglobulin and mucin domain- | TIMD4 TIM4 | turquoise |
| | containing 4 | | |
| TIMP1 | Metalloproteinase inhibitor 1 | TIMP1 CLGI TIMP | turquoise |
| TINAGL1 | Tubulointerstitial nephritis antigen-like | TINAGL1 GIS5 | turquoise |
| | | LCN7 OLRG2 | |
| | | TINAGL PP6614 | |
| | | PSEC0088 | |
| | | UNQ204/PRO230 | |
| TMSB10 | Thymosin beta-10 | TMSB10 PTMB10 | turquoise |
| | | THYB10 | |
| TNC | Tenascin (TN) (Cytotactin) (GMEM) (GP | TNC HXB | turquoise |
| | 150-225) | | |
| TNF | Tumor necrosis factor (Cachectin) (TNF- | TNF TNFA | turquoise |
| | alpha) | TNFSF2 | |
| TNFRSF10A | Tumor necrosis factor receptor superfamily | TNFRSF10A APO2 | turquoise |
| | member 10a | DR4 TRAILR1 | |
| TNFRSF10B | Tumor necrosis factor receptor superfamily | TNFRSF10B DR5 | turquoise |
| | member 10b | KILLER TRAILR2 | |
| | | TRICK2 ZTNFR9 | |
| | | UNQ160/PRO186 | |
| TNFRSF10C | Tumor necrosis factor receptor superfamily | TNFRSF10C DCR1 | turquoise |
| | member 10c | LIT TRAILR3 | |
| | | TRID | |
| | | UNQ321/PRO366 | |
| TNFRSF11A | Tumor necrosis factor receptor superfamily | TNFRSF11A | turquoise |
| | member 11a | RANK | |
| TNFRSF12A | Tumor necrosis factor receptor superfamily | TNFRSF12A FN14 | turquoise |
| | member 12a | | |
| TNFRSF14 | Tumor necrosis factor receptor superfamily | TNFRSF14 HVEA | turquoise |
| | member 14 | HVEM | |
| | | UNQ329/PRO509 | |
| TNFRSF19 | Tumor necrosis factor receptor superfamily | TNFRSF19 TAJ | turquoise |
| | member 19 | TROY | |
| | | UNQ1888/PRO4333 | |
| TNFRSF1A | Tumor necrosis factor receptor superfamily | TNFRSF1A TNFAR | turquoise |
| | member 1a | TNFR1 | |
| TNFRSF1B | Tumor necrosis factor receptor superfamily | TNFRSF1B TNFBR | turquoise |
| | member 1b | TNFR2 | |
| TNFRSF21 | Tumor necrosis factor receptor superfamily | TNFRSF21 DR6 | turquoise |
| | member 21 | UNQ437/PRO868 | |
| TNFRSF4 | Tumor necrosis factor receptor superfamily | TNFRSF4 TXGP1L | turquoise |
| | member 4 | | |
| TNFRSF6B | Tumor necrosis factor receptor superfamily | TNFRSF6B DCR3 | turquoise |
| | member 6b | TR6 | |
| | | UNQ186/PRO212 | |
| TNFSF13 | Tumor necrosis factor ligand superfamily | TNFSF13 APRIL | turquoise |
| | member 13 | TALL2 ZTNF2 | |
| | | UNQ383/PRO715 | |
| TNR | Tenascin-R (TN-R) (Janusin) (Restrictin) | TNR | turquoise |
| TPSAB1 | Tryptase alpha/beta-1 (Tryptase-1) (EC | TPSAB1 TPS1 | turquoise |
| | 3.4.21.59) | TPS2 TPSB1 | |
| TREM2 | Triggering receptor expressed on myeloid | TREM2 | turquoise |
| | cells 2 ( | | |
| TRIAP1 | TP53-regulated inhibitor of apoptosis 1 | TRIAP1 15E1.1 | turquoise |
| | (Protein 1 | HSPC132 | |
| TXNDC15 | Thioredoxin domain-containing protein 15 | TXNDC15 C5orf14 | turquoise |
| | | UNQ335/PRO534 | |
| TXNDC5 | Thioredoxin domain-containing protein 5 | TXNDC5 TLP46 | turquoise |
| | (Endoplasm | UNQ364/PRO700 | |
| ULBP2 | UL16-binding protein 2 (ALCAN-alpha) | ULBP2 N2DL2 | turquoise |
| | (NKG2D ligand | RAET1H | |
| | | UNQ463/PRO791 | |
| VAMP5 | Vesicle-associated membrane protein 5 | VAMP5 HSPC191 | turquoise |
| | (VAMP-5) (My | | |
| VASN | Vasorin (Protein slit-like 2) | VASN SLITL2 | turquoise |
| | | UNQ314/PRO357/ | |
| | | PRO1282 | |
| VCAM1 | Vascular cell adhesion protein 1 (V-CAM 1) | VCAM1 | turquoise |
| | (VCAM-1 | | |
| VCAN | Versican core protein (Chondroitin sulfate | VCAN CSPG2 | turquoise |
| | proteog | | |
| VEGFD | Vascular endothelial growth factor D | VEGFD FIGF | turquoise |
| | (VEGF-D) (c-F | | |
| VMO1 | Vitelline membrane outer layer protein 1 | VMO1 | turquoise |
| | homolog | UNQ6350/PRO21055 | |
| VSIG4 | V-set and immunoglobulin domain- | VSIG4 CRIg Z39IG | turquoise |
| | containing protein | UNQ317/PRO362 | |
| VWC2 | Brorin (Brain-specific chordin-like protein) | VWC2 | turquoise |
| | (von | UNQ739/PRO1434 | |
| VWF | von Willebrand factor (vWF) [Cleaved into: | VWF F8VWF | turquoise |
| | von Wil | | |
| WFDC12 | WAP four-disulfide core domain protein 12 | WFDC12 | turquoise |
| | (Putativ | C20orf122 WAP2 | |
| | | UNQ544/PRO844 | |
| WFDC2 | WAP four-disulfide core domain protein 2 | WFDC2 HE4 | turquoise |
| | (Epididym | WAP5 | |
| WFIKKN2 | WAP, Kazal, immunoglobulin, Kunitz and | WFIKKN2 GASP1 | turquoise |
| | NTR domain- | WFIKKNRP | |
| | | UNQ9235/PRO31996 | |
| WISP1 | CCN family member 4 (WNT1-inducible- | CCN4 WISP1 | turquoise |
| | signaling path | | |
| WISP2 | CCN family member 5 (Connective tissue | CCN5 CT58 | turquoise |
| | growth fact | CTGFL WISP2 | |
| | | UNQ228/PRO261 | |
| WNT9A | Protein Wnt-9a (Protein Wnt-14) | WNT9A WNT14 | turquoise |
| XG | Glycoprotein Xg (Protein PBDX) | XG PBDX | turquoise |
| ADA | Adenosine deaminase (EC 3.5.4.4) | ADA ADA1 | blue |
| | (Adenosine aminoh | | |
| ADAMTS13 | A disintegrin and metalloproteinase with | ADAMTS13 C9orf8 | blue |
| | thrombosp | UNQ6102/PRO20085 | |
| AMFR | E3 ubiquitin-protein ligase AMFR (EC | AMFR RNF45 | blue |
| | 2.3.2.27) (Au | | |
| ANXA5 | Annexin A5 (Anchorin CII) (Annexin V) | ANXA5 ANX5 | blue |
| | (Annexin-5) | ENX2 PP4 | |
| ARHGAP1 | Rho GTPase-activating protein 1 (CDC42 | ARHGAP1 | blue |
| | GTPase-acti | CDC42GAP | |
| | | RHOGAP1 | |
| ARID4B | AT-rich interactive domain-containing | ARID4B BRCAA1 | blue |
| | protein 4B ( | RBBP1L1 RBP1L1 | |
| | | SAP180 | |
| ATP5IF1 | ATPase inhibitor, mitochondrial (ATP | ATP5IF1 ATPI | blue |
| | synthase F1 s | ATPIF1 | |
| ATP6AP2 | Renin receptor (ATPase H(+)-transporting | ATP6AP2 ATP6IP2 | blue |
| | lysosomal | CAPER ELDF10 | |
| | | HT028 MSTP009 | |
| | | PSEC0072 | |
| ATP6V1F | V-type proton ATPase subunit F (V-ATPase | ATP6V1F ATP6S14 | blue |
| | subunit F | VATF | |
| ATXN10 | Ataxin-10 (Brain protein E46 homolog) | ATXN10 SCA10 | blue |
| | (Spinocerebe | | |
| BAX | Apoptosis regulator BAX (Bcl-2-like | BAX BCL2L4 | blue |
| | protein 4) (Bc | | |
| BLMH | Bleomycin hydrolase (BH) (BLM | BLMH | blue |
| | hydrolase) (BMH) (EC | | |
| BPIFB1 | BPI fold-containing family B member 1 | BPIFB1 C20orf114 | blue |
| | (Long palate | LPLUNC1 | |
| | | UNQ706/PRO1357 | |
| BRK1 | Protein BRICK1 (BRK1) | BRK1 C3orf10 | blue |
| | | HSPC300 MDS027 | |
| C4BPB | C4b-binding protein beta chain | C4BPB | blue |
| CA6 | Carbonic anhydrase 6 (EC 4.2.1.1) | CA6 | blue |
| | (Carbonate dehyd | | |
| CA9 | Carbonic anhydrase 9 (EC 4.2.1.1) | CA9 G250 MN | blue |
| | (Carbonate dehyd | | |
| CANT1 | Soluble calcium-activated nucleotidase 1 | CANT1 SHAPY | blue |
| | (SCAN-1) | | |
| CBLN4 | Cerebellin-4 (Cerebellin-like glycoprotein | CBLN4 CBLNL1 | blue |
| | 1) | UNQ718/PRO1382 | |
| CCDC80 | Coiled-coil domain-containing protein 80 | CCDC80 DRO1 | blue |
| | (Down-reg | URB HBE245 | |
| CCL7 | C-C motif chemokine 7 (Monocyte | CCL7 MCP3 | blue |
| | chemoattractant pr | SCYA6 SCYA7 | |
| CD22 | B-cell receptor CD22 (B-lymphocyte cell | CD22 SIGLEC2 | blue |
| | adhesion m | | |
| CD27 | CD27 antigen (CD27L receptor) (T-cell | CD27 TNFRSF7 | blue |
| | activation a | | |
| CD40 | Tumor necrosis factor receptor superfamily | CD40 TNFRSF5 | blue |
| | member | | |
| CD70 | CD70 antigen (CD27 ligand) (CD27-L) | CD70 CD27L | blue |
| | (Tumor necrosi | CD27LG TNFSF7 | |
| CD79B | B-cell antigen receptor complex-associated | CD79B B29 IGB | blue |
| | protein | | |
| CD83 | CD83 antigen (hCD83) (B-cell activation | CD83 | blue |
| | protein) ( | | |
| CHEK2 | Serine/threonine-protein kinase Chk2 (EC | CHEK2 CDS1 | blue |
| | 2.7.11.1) | CHK2 RAD53 | |
| CLPP | ATP-dependent Clp protease proteolytic | CLPP | blue |
| | subunit, mi | | |
| CLSPN | Claspin (hClaspin) | CLSPN | blue |
| CLTA | Clathrin light chain A (Lca) | CLTA | blue |
| CNPY4 | Protein canopy homolog 4 | CNPY4 PSEC0237 | blue |
| | | UNQ1909/PRO4354 | |
| COX5B | Cytochrome c oxidase subunit 5B, | COX5B | blue |
| | mitochondrial (Cy | | |
| CXCL8 | Interleukin-8 (IL-8) (C-X-C motif | CXCL8 IL8 | blue |
| | chemokine 8) (Ch | | |
| CXCL9 | C-X-C motif chemokine 9 (Gamma- | CXCL9 CMK MIG | blue |
| | interferon-induced | SCYB9 | |
| DCTPP1 | dCTP pyrophosphatase 1 (EC 3.6.1.12) | DCTPP1 XTP3TPA | blue |
| | (Deoxycytidin | CDA03 | |
| DFFA | DNA fragmentation factor subunit alpha | DFFA DFF1 DFF45 | blue |
| | (DNA fragme | H13 | |
| DNPH1 | 2′-deoxynucleoside 5′-phosphate N- | DNPH1 C6orf108 | blue |
| | hydrolase 1 (EC | RCL | |
| DPY30 | Protein dpy-30 homolog (Dpy-30-like | DPY30 | blue |
| | protein) (Dpy- | | |
| DTX3 | Probable E3 ubiquitin-protein ligase DTX3 | DTX3 RNF154 | blue |
| | (EC 2.3. | | |
| EIF4EBP1 | Eukaryotic translation initiation factor 4E- | EIF4EBP1 | blue |
| | bindin | | |
| ELOA | Elongin-A (EloA) (Elongin 110 kDa | ELOA TCEB3 | blue |
| | subunit) (RNA po | MSTP059 | |
| ENO2 | Gamma-enolase (EC 4.2.1.11) (2-phospho- | ENO2 | blue |
| | D-glycerate | | |
| ERP44 | Endoplasmic reticulum resident protein 44 | ERP44 KIAA0573 | blue |
| | (ER prot | TXNDC4 | |
| | | UNQ532/PRO1075 | |
| EZR | Ezrin (Cytovillin) (Villin-2) (p81) | EZR VIL2 | blue |
| FABP5 | Fatty acid-binding protein 5 (Epidermal- | FABP5 | blue |
| | type fatty | | |
| FCRL1 | Fc receptor-like protein 1 (FcR-like protein | FCRL1 FCRH1 | blue |
| | 1) (F | IFGP1 IRTA5 | |
| FCRL2 | Fc receptor-like protein 2 (FcR-like protein | FCRL2 FCRH2 | blue |
| | 2) (F | IFGP4 IRTA4 | |
| | | SPAP1 | |
| | | UNQ9236/PRO31998 | |
| FCRL3 | Fc receptor-like protein 3 (FcR-like protein | FCRL3 FCRH3 | blue |
| | 3) (F | IFGP3 IRTA3 | |
| | | SPAP2 | |
| FKBP4 | Peptidyl-prolyl cis-trans isomerase FKBP4 | FKBP4 FKBP52 | blue |
| | (PPIase | | |
| FOPNL | Centrosomal protein 20 (FGFR1OP N- | CEP20 C16orf63 | blue |
| | terminal-like pr | FOPNL FOR20 | |
| | | PHSECRG2 | |
| FOSB | Protein fosB (G0/G1 switch regulatory | FOSB G0S3 | blue |
| | protein 3) | | |
| FURIN | Furin (EC 3.4.21.75) (Dibasic-processing | FURIN FUR PACE | blue |
| | enzyme) ( | PCSK3 | |
| FUS | RNA-binding protein FUS (75 kDa DNA- | FUS TLS | blue |
| | pairing protei | | |
| FXN | Frataxin, mitochondrial (EC 1.16.3.1) | FXN FRDA X25 | blue |
| | (Friedreich | | |
| GBP2 | Guanylate-binding protein 2 (EC 3.6.5.-) | GBP2 | blue |
| | (GTP-bind | | |
| GFER | FAD-linked sulfhydryl oxidase ALR (EC | GFER ALR HERV 1 | blue |
| | 1.8.3.2) (Au | HPO | |
| GLOD4 | Glyoxalase domain-containing protein 4 | GLOD4 C17orf25 | blue |
| | | CGI-150 My027 | |
| GNE | Bifunctional UDP-N-acetylglucosamine 2- | GNE GLCNE | blue |
| | epimerase/N | | |
| GPKOW | G-patch domain and KOW motifs- | GPKOW GPATC5 | blue |
| | containing protein ( | GPATCH5 T54 | |
| GRN | Progranulin (PGRN) (Acrogranin) | GRN | blue |
| | (Epithelin precurs | | |
| GRPEL1 | GrpE protein homolog 1, mitochondrial | GRPEL1 GREPEL1 | blue |
| | (HMGE) (Mt-G | | |
| GSTP1 | Glutathione S-transferase P (EC 2.5.1.18) | GSTP1 FAEES3 | blue |
| | (GST cla | GST3 | |
| HDGF | Hepatoma-derived growth factor (HDGF) | HDGF HMG1L2 | blue |
| | (High mobili | | |
| HEXIM1 | Protein HEXIM1 (Cardiac lineage protein 1) | HEXIM1 CLP1 | blue |
| | (Estrog | EDG1 HIS1 MAQ1 | |
| HLA-DRA | HLA class II histocompatibility antigen, DR | HLA-DRA HLA- | blue |
| | alpha | DRA1 | |
| HMOX2 | Heme oxygenase 2 (HO-2) (EC 1.14.14.18) | HMOX2 HO2 | blue |
| HTRA2 | Serine protease HTRA2, mitochondrial (EC | HTRA2 OMI | blue |
| | 3.4.21.10 | PRSS25 | |
| IGSF3 | Immunoglobulin superfamily member 3 | IGSF3 EWI3 | blue |
| | (IgSF3) (Glu-T | KIAA0466 | |
| IL16 | Pro-interleukin-16 [Cleaved into: | IL16 | blue |
| | Interleukin-16 ( | | |
| IL1RL1 | Interleukin-1 receptor-like 1 (EC 3.2.2.6) | IL1RL1 DER4 ST2 | blue |
| | (Protei | T1 | |
| IL2RA | Interleukin-2 receptor subunit alpha (IL-2 | IL2RA | blue |
| | recepto | | |
| IL6 | Interleukin-6 (IL-6) (B-cell stimulatory | IL6 IFNB2 | blue |
| | factor 2) | | |
| ING1 | Inhibitor of growth protein 1 | ING1 | blue |
| INPP1 | Inositol polyphosphate 1-phosphatase (IPP) | INPP1 | blue |
| ITGB1BP1 | Integrin beta-1-binding protein 1 | ITGB1BP1 ICAP1 | blue |
| JUN | Transcription factor AP-1 (Activator protein | JUN | blue |
| | 1) | | |
| KIRREL2 | Kin of IRRE-like protein 2 | KIRREL2 NEPH3 | blue |
| | | UNQ5827/PRO19646 | |
| KITLG | Kit ligand (Mast cell growth factor) (MGF) | KITLG MGF SCF | blue |
| KYAT1 | Kynurenine--oxoglutarate transaminase 1 | KYAT1 CCBL1 | blue |
| KYNU | Kynureninase (EC 3.7.1.3) (L-kynurenine | KYNU | blue |
| | hydrolase) | | |
| LAP3 | leucine aminopeptidase 3 | LAP3 LAPEP PEPS | blue |
| LBP | Lipopolysaccharide-binding protein (LBP) | LBP | blue |
| LEPR | Leptin receptor (LEP-R) (HuB219) | LEPR DB OBR | blue |
| LIF | Leukemia inhibitory factor (LIF) | LIF HILDA | blue |
| LRIG1 | leucine rich repeats and immunoglobulin | LRIG1 LIG1 | blue |
| | like domains 1 | | |
| LRMP | Inositol 1,4,5-triphosphate receptor | IRAG2 JAW1 | blue |
| | associated 2 | LRMP | |
| LRPAP1 | LDL receptor related protein associated | LRPAP1 A2MRAP | blue |
| | protein 1 | | |
| LRRN1 | Leucine-rich repeat neuronal protein 1 | LRRN1 KIAA1497 | blue |
| | | Nbla10449 | |
| | | UNQ693/PRO1338 | |
| LTA | Lymphotoxin-alpha (LT-alpha) | LTA TNFB | blue |
| | | TNFSF1 | |
| LTA4H | Leukotriene A-4 hydrolase (LTA-4 | LTA4H LTA4 | blue |
| | hydrolase) | | |
| LY9 | T-lymphocyte surface antigen Ly-9 | LY9 CDABP0070 | blue |
| LYAR | Cell growth-regulating nucleolar protein | LYAR PNAS-5 | blue |
| LYN | Tyrosine-protein kinase Lyn | LYN JTK8 | blue |
| MAD1L1 | Mitotic spindle assembly checkpoint protein | MAD1L1 MAD1 | blue |
| | MAD1 | TXBP181 | |
| MAGED1 | Melanoma-associated antigen D1 | MAGED1 NRAGE | blue |
| | | PP2250 PRO2292 | |
| MAPK9 | Mitogen-activated protein kinase 9 (MAP | MAPK9 JNK2 | blue |
| | kinase 9) | PRKM9 SAPK1A | |
| METAP1D | Methionine aminopeptidase 1D | METAP1D MAP1D | blue |
| MGMT | Methylated-DNA--protein-cysteine | MGMT | blue |
| | methyltransferase | | |
| MMP12 | Macrophage metalloelastase (MME) | MMP12 HME | blue |
| MPHOSPH8 | M-phase phosphoprotein 8 | MPHOSPH8 MPP8 | blue |
| MPI | Mannose-6-phosphate isomerase | MPI PMI1 | blue |
| MRPL46 | 39S ribosomal protein L46, mitochondrial | MRPL46 C15orf4 | blue |
| | (L46mt) | LIECG2 | |
| MSTN | Growth/differentiation factor 8 (GDF-8) | MSTN GDF8 | blue |
| MUC16 | Mucin-16 (MUC-16) | MUC16 CA125 | blue |
| MZB1 | Marginal zone B- and B1-cell-specific | MZB1 MEDA7 | blue |
| | protein | PACAP HSPC190 | |
| NDUFS6 | NADH dehydrogenase [ubiquinone] iron- | NDUFS6 | blue |
| | sulfur protein subunit S6 | | |
| NEFL | Neurofilament light polypeptide (NF-L) | NEFL NF68 NFL | blue |
| NFKBIE | NF-kappa-B inhibitor epsilon (NF-kappa- | NFKBIE IKBE | blue |
| | BIE) | | |
| NINJ1 | Ninjurin-1 (Nerve injury-induced protein 1) | NINJ1 | blue |
| NUDC | Nuclear migration protein nudC | NUDC | blue |
| NUDT5 | ADP-sugar pyrophosphatase | NUDT5 NUDIX5 | blue |
| | | HSPC115 | |
| OGFR | Opioid growth factor receptor (OGFr) | OGFR | blue |
| P4HB | Protein disulfide-isomerase (PDI) | P4HB ERBA2L PDI | blue |
| | | PDIA1 PO4DB | |
| PADI2 | Protein-arginine deiminase type-2 | PADI2 KIAA0994 | blue |
| | | PAD2 PDI2 | |
| PAEP | Glycodelin (GD) (Placental protein 14) | PAEP | blue |
| | (PP14) | | |
| PAG1 | phosphoprotein membrane anchor with | PAG1 CBP PAG | blue |
| | glycosphingolipid microdomains 1 | | |
| PARP1 | Poly [ADP-ribose] polymerase 1 | PARP1 ADPRT | blue |
| | | PPOL | |
| PDCD5 | Programmed cell death protein 5 | PDCD5 TFAR19 | blue |
| PDCD6 | Programmed cell death protein 6 | PDCD6 ALG2 | blue |
| PFDN2 | Prefoldin subunit 2 | PFDN2 PFD2 | blue |
| | | HSPC231 | |
| PLA2G2A | Phospholipase A2, membrane associated | PLA2G2A PLA2B | blue |
| | | PLA2L RASF-A | |
| PLIN3 | Perilipin-3 | PLIN3 M6PRBP1 | blue |
| | | TIP47 | |
| PODXL2 | Podocalyxin-like protein 2 (Endoglycan) | PODXL2 | blue |
| | | UNQ1861/PRO3742 | |
| POLR2F | DNA-directed RNA polymerases I, II, and | POLR2F POLRF | blue |
| | III subunit F | | |
| PON2 | Serum paraoxonase/arylesterase 2 (PON 2) | PON2 | blue |
| PON3 | Serum paraoxonase/lactonase 3 | PON3 | blue |
| PPCDC | Phosphopantothenoylcysteine decarboxylase | PPCDC COAC | blue |
| | (PPC-DC) | MDS018 | |
| | | UNQ9365/PRO34154 | |
| PPP1R2 | Protein phosphatase inhibitor 2 (IPP-2) | PPP1R2 IPP2 | blue |
| PPP3R1 | protein phosphatase 3 regulatory subunit B, | PPP3R1 CNA2 | blue |
| | alpha | CNB | |
| PQBP1 | Polyglutamine-binding protein 1 (PQBP-1) | PQBP1 NPW38 | blue |
| | | JM26 | |
| PRDX1 | Peroxiredoxin-1 | PRDX1 PAGA | blue |
| | | PAGB TDPX2 | |
| PRDX3 | peroxiredoxin 3 | PRDX3 AOP1 | blue |
| PRDX6 | Peroxiredoxin-6 | PRDX6 AOP2 | blue |
| | | KIAA0106 | |
| PREB | Prolactin regulatory element-binding protein | PREB SEC12 | blue |
| PSIP1 | PC4 and SFRS1-interacting protein | PSIP1 DFS70 | blue |
| | | LEDGF PSIP2 | |
| PSMD9 | 26S proteasome non-ATPase regulatory | PSMD9 | blue |
| | subunit 9 | | |
| PSME1 | Proteasome activator complex subunit 1 | PSME1 IFI5111 | blue |
| PSME2 | Proteasome activator complex subunit 2 | PSME2 | blue |
| PSMG3 | Proteasome assembly chaperone 3 (PAC-3) | PSMG3 C7orf48 | blue |
| | (hPAC3) | PAC3 | |
| PTS | 6-pyruvoyl tetrahydrobiopterin synthase | PTS | blue |
| PTX3 | Pentraxin-related protein PTX3 | PTX3 TNFAIP5 | blue |
| | | TSG14 | |
| QPCT | Glutaminyl-peptide cyclotransferase | QPCT | blue |
| RABEPK | Rab9 effector protein with kelch motifs | RABEPK RAB9P40 | blue |
| RABGAP1L | Rab GTPase-activating protein 1-like | RABGAP1L HHL | blue |
| | | KIAA0471 | |
| RAD23B | UV excision repair protein RAD23 homolog | RAD23B | blue |
| | B (HR23B) | | |
| RCOR1 | REST corepressor 1 (Protein CoREST) | RCOR1 KIAA0071 | blue |
| | | RCOR | |
| RP2 | Protein XRP2 | RP2 | blue |
| RRM2 | Ribonucleoside-diphosphate reductase | RRM2 RR2 | blue |
| | subunit M2 | | |
| RRM2B | Ribonucleoside-diphosphate reductase | RRM2B P53R2 | blue |
| | subunit M2 B | | |
| SCRN1 | Secernin-1 | SCRN1 KIAA0193 | blue |
| SEMA4C | Semaphorin-4C | SEMA4C | blue |
| | | KIAA1739 SEMAI | |
| | | UNQ5855/PRO34487 | |
| SEPTIN9 | Septin-9 (MLL septin-like fusion protein | SEPTIN9 | blue |
| | MSF-A) | KIAA0991 MSF | |
| | | SEPT9 | |
| SERPINB8 | Serpin B8 (Cytoplasmic antiproteinase 2) | SERPINB8 PI8 | blue |
| | (CAP-2) | | |
| SERPINB9 | Serpin B9 (Cytoplasmic antiproteinase 3) | SERPINB9 PI9 | blue |
| | (CAP-3) | | |
| SETMAR | Histone-lysine N-methyltransferase | SETMAR | blue |
| | SETMAR | | |
| SF3B4 | Splicing factor 3B subunit 4 | SF3B4 SAP49 | blue |
| SIT1 | Signaling threshold-regulating | SIT1 SIT | blue |
| | transmembrane adaptor 1 | | |
| SLAMF6 | SLAM family member 6 | SLAMF6 KALI | blue |
| | | UNQ6123/PRO20080 | |
| SLC16A1 | Monocarboxylate transporter 1 (MCT 1) | SLC16A1 MCT1 | blue |
| SMARCA2 | SWI/SNF related, matrix associated, actin | SMARCA2 | blue |
| | dependent regulator of chromatin, subfamily | BAF190B BRM | |
| | a, member 2 | SNF2A SNF2L2 | |
| SOD1 | Superoxide dismutase 1 | SOD1 | blue |
| SOD2 | Superoxide dismutase 2 | SOD2 | blue |
| SPINT1 | Kunitz-type protease inhibitor 1 | SPINT1 HAI1 | blue |
| | | UNQ223/PRO256 | |
| SRPK2 | SRSF protein kinase 2 | SRPK2 | blue |
| SSB | small RNA binding exonuclease protection | SSB | blue |
| | factor La | | |
| ST3GAL1 | ST3 beta-galactoside alpha-2,3- | ST3GAL1 SIAT4 | blue |
| | sialyltransferase 1 | SIAT4A | |
| STK11 | Serine/threonine-protein kinase STK11 | STK11 LKB1 PJS | blue |
| TBL1X | F-box-like/WD repeat-containing protein | TBL1X TBL1 | blue |
| | TBL1X | | |
| TCL1A | T-cell leukemia/lymphoma protein 1A | TCL1A TCL1 | blue |
| TFPI2 | Tissue factor pathway inhibitor 2 (TFPI-2) | TFPI2 | blue |
| THOP1 | Thimet oligopeptidase | THOP1 | blue |
| TIGAR | Fructose-2,6-bisphosphatase TIGAR | TIGAR C12orf5 | blue |
| TNFRSF11B | Tumor necrosis factor receptor superfamily | TNFRSF11B OCIF | blue |
| | member 11b | OPG | |
| TNFRSF13C | Tumor necrosis factor receptor superfamily | TNFRSF13C | blue |
| | member 13c | BAFFR BR3 | |
| TNFRSF9 | Tumor necrosis factor receptor superfamily | TNFRSF9 CD137 | blue |
| | member 9 | ILA | |
| TP53 | Cellular tumor antigen p53 (Antigen NY- | TP53 P53 | blue |
| | CO-13) | | |
| TXLNA | Alpha-taxilin | TXLNA TXLN | blue |
| TXNRD1 | Thioredoxin reductase 1, cytoplasmic (TR) | TXNRD1 GRIM12 | blue |
| | | KDRF | |
| TYMP | Thymidine phosphorylase (TP) | TYMP ECGF1 | blue |
| VWA1 | von Willebrand factor A domain-containing | VWA1 | blue |
| | protein | | |
| WARS | Tryptophan--tRNA ligase | WARS1 IFI53 | blue |
| | | WARS WRS | |
| XRCC4 | DNA repair protein XRCC4 | XRCC4 | blue |
| ZBTB17 | Zinc finger and BTB domain-containing | ZBTB17 MIZ1 | blue |
| | protein 17 | ZNF151 Z | |
| ACAA1 | 3-ketoacyl-CoA thiolase, peroxisomal | ACAA1 ACAA | black |
| | | PTHIO | |
| ACY1 | Aminoacylase-1 (ACY-1) | ACY1 | black |
| ADH4 | All-trans-retinol dehydrogenase [NAD(+)] | ADH4 | black |
| | ADH4 | | |
| AGXT | Serine--pyruvate aminotransferase (SPT) | AGXT AGT1 SPAT | black |
| AIFM1 | Apoptosis-inducing factor 1, mitochondrial | AIFM1 AIF PDCD8 | black |
| AKR1C4 | Aldo-keto reductase family 1 member C4 | AKR1C4 CHDR | black |
| ALDH1A1 | Retinal dehydrogenase 1 (RALDH 1) | ALDH1A1 ALDC | black |
| | (RalDH1) | ALDH1 PUMB1 | |
| ALDH3A1 | aldehyde dehydrogenase 3 family member | ALDH3A1 ALDH3 | black |
| | A1 | | |
| BAIAP2 | Brain-specific angiogenesis inhibitor 1- | BAIAP2 | black |
| | associated | | |
| C19orf12 | Protein C19orf12 | C19orf12 | black |
| CA5A | Carbonic anhydrase 5A, mitochondrial | CA5A CA5 | black |
| CES1 | Liver carboxylesterase 1 | CES1 CES2 SES1 | black |
| DCXR | L-xylulose reductase (XR) | DCXR SDR20C1 | black |
| DDAH1 | N(G),N(G)-dimethylarginine | DDAH1 DDAH | black |
| | dimethylaminohydrolase | | |
| DECR1 | 2,4-dienoyl-CoA reductase, mitochondrial | DECR1 DECR | black |
| | | SDR18C1 | |
| FABP1 | Fatty acid-binding protein 1 | FABP1 FABPL | black |
| FBP1 | Fructose-1,6-bisphosphatase 1 (FBPase 1) | FBP1 FBP | black |
| GSTA1 | Glutathione S-transferase A1 | GSTA1 | black |
| GSTA3 | Glutathione S-transferase A3 | GSTA3 | black |
| HAO1 | Hydroxyacid oxidase 1 (HAOX1) | HAO1 GOX1 | black |
| | | HAOX1 | |
| HNMT | Histamine N-methyltransferase (HMT) | HNMT | black |
| IL32 | Interleukin-32 (IL-32) | IL32 NK4 TAIF | black |
| KRT18 | Keratin, type I cytoskeletal 18 | KRT18 CYK18 | black |
| | | PIG46 | |
| LHPP | Phospholysine phosphohistidine inorganic | LHPP | black |
| | pyrophosphatase | | |
| MME | membrane metalloendopeptidase | MME EPN | black |
| MVK | Mevalonate kinase (MK) | MVK | black |
| NFATC3 | Nuclear factor of activated T-cells, | NFATC3 NFAT4 | black |
| | cytoplasmic 3 | | |
| PBLD | Phenazine biosynthesis-like domain- | PBLD MAWBP | black |
| | containing protein | | |
| QDPR | Dihydropteridine reductase | QDPR DHPR | black |
| | | SDR33C1 | |
| RBKS | Ribokinase (RK) | RBKS RBSK | black |
| RBP5 | Retinol-binding protein 5 | RBP5 | black |
| SCLY | Selenocysteine lyase (hSCL) | SCLY SCL | black |
| SCP2 | Non-specific lipid-transfer protein | SCP2 | black |
| SHMT1 | Serine hydroxymethyltransferase, cytosolic | SHMT1 | black |
| | (SHMT) | | |
| SORD | Sorbitol dehydrogenase (SDF) | SORD | black |
| SULT2A1 | Sulfotransferase 2A1 (ST2A1) | SULT2A1 HST | black |
| | | STD | |
| TST | Thiosulfate sulfurtransferase | TST | black |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

1. A method of determining a risk of an adverse event upon administration of an immunotherapy to a cancer subject in need thereof, the method comprising:
quantifying a baseline serum level from the cancer subject of one or more biomarkers selected from REG3A, KYNU, OSMR, NELL2, and MET; or
quantifying a day 0 serum level from the cancer subject of one or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5; and
determining the risk of the adverse event in the cancer subject based on the step of quantifying the baseline serum level of the one or more biomarkers or the step of quantifying the day 0 serum level from the cancer subject of the one or more biomarkers,
wherein at least one of an increased baseline serum level of the one or more biomarkers versus a control baseline serum level of the one or more biomarkers and/or an increased day 0 serum level of the one or more biomarkers versus a control day 0 serum level of the one or more biomarkers is correlated with an increased risk of the adverse event in the cancer subject,
wherein the adverse event is one or more of an immune activation induced stress, a Grade 3+ CRS, and a Grade 3+ NE.

2. The method of claim 1, wherein the method comprises
quantifying a baseline serum level from the cancer subject of one or more biomarkers selected from REG3A, KYNU, OSMR, NELL2, and MET; and
quantifying a day 0 serum level from the cancer subject of one or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5; and
determining the risk of the adverse event in the cancer subject based on the step of quantifying the baseline serum level of the one or more biomarkers and the step of quantifying the day 0 serum level from the cancer subject of the one or more biomarkers.

3. The method of claim 1, wherein the method comprises quantifying a baseline serum level from the cancer subject of two or more biomarkers selected from REG3A, KYNU, OSMR, NELL2, and MET.

4. The method of claim 1, wherein the method comprises quantifying a day 0 serum level from the cancer subject of two or more biomarkers selected from SOD2, VAMP5, PCDH17, ACE2, REG1B, REG3A, AREG, CELA3A, CEACAM1, STK11, LY9, LY96, CKAP4, CXCL1, EIF5A, IL1A, KIFBP, KIRREL2, NUB1, PAG1, PCDH17, TGFB1, ADAM15, BSG, HLA-DRA, ICAM2, OSMR, SERPINB9, CCL16, MEGF9, and MFAP5.

5. The method of claim 1, wherein the control baseline serum level of the one or more biomarkers is a historically observed baseline serum level of the one or more biomarkers not previously observed to be associated with an onset of the adverse event, and wherein the control day 0 serum level of the one or more biomarkers is a historically observed day 0 serum level of the one or more biomarkers not previously observed to be associated an onset of the adverse event.

6. The method of claim 1, wherein the cancer subject is determined to have an increased risk of the adverse event if the baseline serum level of one or more biomarkers selected from MET, OSMR, NELL2, REG3A, KYNU and the day 0 serum level of one or more biomarkers selected from AREG, CKAP4, CXCL1, EIF5A, IL1A, KIFBP, KIRREL2, NUB1, PAG1, PCHD17, STK11, TGFB1, ACE2, ADAM15, BSG, CEACAM1, HLA-DRA, ICAM2, LY9, LY96, OSMR, REG3A, SERPINB9, SOD2, VAMP5, CCL16, CELA3A, MEGF9, MFAP3, REG1B is above a predetermined normalized protein expression range for the one or more biomarkers,
wherein the predetermined normalized protein expression range for the one or more biomarkers are 0.56-0.75 for MET, 0.62-0.77 for OSMR (baseline), 0.14-0.50 for NELL2, 1.80-2.19 for REG3A (baseline), 1.60-1.98 for KYNU, 0.84-1.25 for CCL16, 1.37-2.01 for CELA3A, 0.26-0.48 for MEGF9, 0.33-0.49 for MFAP3, 1.49-1.99 for REG1B, 0.88-1.22 for AREG, 0.73-0.96 for BSG, 1.83-2.31 for CKAP4, 3.48-3.91 for CXCL1, 0.09-0.23 for EIF5A, 0.46-0.81 for IL1A, 1.36-1.66 for KIFBP, 1.62-2.13 for KIRREL2, 1.39-1.77 for NUB1, 0.65-0.79 for OSMR (day 0), 2.16-2.74 for PAG1, 1.03-1.32 for PCDH17, 1.09-1.35 for STK11, 1.20-1.65 for ACE2, 1.09-1.31 for ADAM15, 0.68-0.90 for CEACAM1, 1.13-1.41 for HLA-DRA, 1.21-1.43 for ICAM2, 0.11-0.49 for LY9, 2.34-2.88 for REG3A (day 0), 2.12-2.30 for SERPINB9, 1.37-1.86 for SOD2, 0.54-0.70 for LY96, 1.51-1.67 for TGFB1, and 1.30-1.73 for VAMP5.

7. The method of claim 1, further comprising one or more of treating the cancer subject with a modified pre-conditioning regimen versus a standard pre-conditioning regimen, not administering the immunotherapy to the cancer subject, administering the immunotherapy to the cancer subject with a modified dose versus a standard dose, or administering the immunotherapy to the cancer subject in combination with an agent for mediating an immune activation induced stress, a Grade 3+ CRS, and/or a Grade 3+ NE, if the cancer subject is determined to have an increased risk of an adverse event.

8. The method of claim 7, wherein the modified pre-conditioning regimen comprises administering to the cancer subject a modified dose of at least one of cyclophosphamide and fludarabine instead of a predetermined dose of cyclophosphamide or fludarabine administered to a control subject.

9. The method of claim 1, further comprising administering the immunotherapy with a combination therapy comprising an agent that reduces cytokine induction and/or endothelial cells disruption if the cancer subject is determined to have an increased risk of an adverse event.

10. The method of claim 9, wherein the agent reduces cytokine induction.

11. The method of claim 10, wherein the agent is administered to the cancer subject prior to administration of the immunotherapy, before a peak expansion of the immunotherapy, or at the peak expansion of the immunotherapy.

12. The method of claim 9, wherein the agent is selected from an anti-IL-1 molecule, a T-cell activation inhibitor, a JAK inhibitor, an anti-GM-CSF molecule, an anti-TNF molecule, an Ang2 inhibitor, an anti-angiogenic therapy, and an anti-IFNg molecule.

13. The method of claim 1, wherein the immunotherapy is a CAR T cell therapy, a TCR T cell therapy, a tumor infiltrating lymphocytes (TIL) cell therapy, or a bispecific T-cell engagers (BiTEs) therapy.

14. The method of claim 13, wherein the immunotherapy is autologous or allogeneic.

15. The method of claim 13, wherein the immunotherapy is CAR T or TCR T cell therapy that recognizes a target antigen.

16. The method of claim 15, wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C(TnC Al), thyroglobulin, tumor stromal antigens, vascular endothelial growth factor receptor-2 (VEGFR2), virus-specific surface antigen such as an HIV-specific antigen (such as HIV gp120), GPC3 (Glypican 3), as well as any derivate or variant of these antigens.

17. The method of claim 1, wherein the cancer subject has a cancer or tumor selected from a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

18. The method of claim 17, wherein the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

19. The method of claim 1, wherein the immunotherapy is selected from axicabtagene ciloleucel, brexucabtagene autoleucel, tisagenlecleucel, lisocabtagene maraleucel, and bb2121.

20. A method of determining a risk of an adverse event upon administration of an immunotherapy to a cancer subject in need thereof, the method comprising:
determining baseline and/or day 0 serum levels of a plurality of biomarkers from the cancer subject;
determining a weight for each of the plurality of biomarkers at least in part by inputting the baseline serum levels of the plurality of biomarkers into an algorithm; and
determining a probability of risk value of the adverse event based at least in part on the weight for each of the plurality of biomarkers,
wherein if the probability of risk value is at least as great as a predetermined cut-off value, then the subject has an increased risk of the adverse event versus a control group, and
wherein the plurality of biomarkers comprise ACE2, IL1A, SERPINB9, and LY96.

21. The method of claim 20, wherein the predetermined cut-off is a Youden cut-off value of between 0.10-0.35.

22. The method of claim 20, wherein the algorithm comprises a logical regression model, a random forest algorithm, or a regularized gradient boosting framework algorithm.
